(19) United States
(12) Patent Application Publication
(10) Pub. No.: US 2025/0145564 A1
(43) Pub. Date: May 8, 2025

(54) SULFONYLUREA COMPOUND, PREPARATION METHOD THEREFOR, AND APPLICATION THEREOF

(71) Applicants: SHANGHAI INSTITUTE OF MATERIA MEDICA, CHINESE ACADEMY OF SCIENCES, Shanghai (CN); TONGJI HOSPITAL AFFILIATED TO TONGJI MEDICAL COLLEGE OF HUAZHONG UNIVERSITY OF SCIENCE & TECHNOLOGY, Hubei (CN)

(72) Inventors: Daowen WANG, Hubei (CN); Xiangrui JIANG, Shanghai (CN); Chen CHEN, Hubei (CN); Hualiang JIANG, Shanghai (CN); Jingshan SHEN, Shanghai (CN)

(21) Appl. No.: 18/684,032

(22) PCT Filed: Aug. 15, 2022
(86) PCT No.: PCT/CN2022/112383

§ 371 Date: Feb. 15, 2024

(30) Foreign Application Priority Data
Aug. 16, 2021 (CN) 202110935522.0

(51) Int. Cl. C07C 311/59; A61K 31/18; A61K 31/4015; A61K 31/403; A61K 31/454; A61K 31/472; A61K 31/4965; A61K 31/55; A61K 31/675; A61P 9/04; C07C 303/40; C07D 207/38; C07D 209/52; C07D 217/24; C07D 223/04; C07D 241/24; C07D 413/12; C07F 9/572

(52) U.S. Cl. CPC C07C 311/59; A61K 31/18; A61K 31/4015; A61K 31/403; A61K 31/454; A61K 31/472; A61K 31/4965; A61K 31/55; A61K 31/675; A61P 9/04; C07C 303/40; C07D 207/38; C07D 209/52; C07D 217/24; C07D 223/04; C07D 241/24; C07D 413/12; C07F 9/572; C07C 2601/14; C07C 2602/42

(57) ABSTRACT

The present invention relates to a sulfonylurea compound represented by formula I, a preparation method therefor, and an application thereof. The compound of the present invention have soluble epoxide hydrolase (sEH) inhibitory activity and can increase the level of epoxyeicosatrienoic acids (EETs)so as to alleviate inflammatory responses, and thus can be used for preventing and treating heart failure. The preparation method of the present invention has the technical advantages of simple steps, high yield, and easily available raw materials.

### CROSS REFERENCE TO RELATED APPLICATIONS

This application claims the priority of the Chinese patent application No. 202110935522.0 filed on Aug. 16, 2021, the disclosure of which is incorporated herein by reference in its entirety.

### FIELD OF THE DISCLOSURE

The present invention belongs to the field of pharmaceutical chemistry and chemical synthesis. Specifically, the present invention relates to a sulfonylurea compound, and preparation method and use thereof.

### BACKGROUND

Heart failure is a condition in which disorders of the contraction and/or relaxation function of the heart results in blood stasis in the venous system and insufficient blood perfusion in the arterial system, thereby causing circulatory disorders in the heart. Myocardium is closely related to the contraction and relaxation function of the heart. Myocardial damage caused by various reasons may lead to heart failure, among which inflammation is one of the important causes for myocardial damage.

Epoxyeicosatrienoic acids (EETs) are a class of functional small molecules generated by arachidonic acid under the action of CYP450. Arachidonic acid is one of the most widely distributed and abundant polyunsaturated essential fatty acid in living organisms. It can generate through the CYP450 pathway four different EETs isomers, namely 5,6-EET, 8,9-EET, 11,12-EET, 14,15-EET, which are collectively known as EETs. EETs have various regulatory effects in the human body, such as increasing sodium-hydrogen reverse transport activity, decreasing cyclooxygenase (COX) activity, and promoting the release of calcium from intracellular storage.

EETs are unstable and can be hydrolyzed by soluble epoxide hydrolase (sEH) to weakly bioactive dihydroxyeicosaterionic acid (DHET), which can be quickly excreted from the body. The CYP-EETs-sEH metabolic pathway plays a regulatory role in multiple key pathways in the human body. Research has shown that sEH inhibitors can reverse endothelial dysfunction, regulate blood pressure, reduce the occurrence of stroke and epilepsy, enhance insulin action, and reduce myocardial damage, and can be anti-inflammatory and analgesic. sEH inhibitors for the treatment of hypertension and diabetes is extensively studied.

In view of this, the present invention is proposed.

### BRIEF SUMMARY

The primary object of the present invention is to provide a sulfonylurea compound of formula I, or a stereoisomer or pharmaceutically acceptable salt thereof.

The second object of the present invention is to provide a method for preparing the sulfonylurea compound.

The third object of the present invention is to provide use of the sulfonylurea compound as a sEH inhibitor in preparation of a medicament for prevention and/or treatment of heart failure.

To achieve the above objects, the following technical solution is adopted.

The present invention provides a sulfonylurea compound of formula I, or a stereoisomer or pharmaceutically acceptable salt thereof,

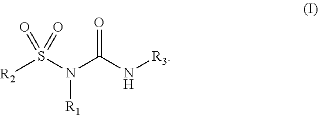

Furthermore, the sulfonylurea compound of formula I of the present invention is selected from the compounds of formulas IA and IB:

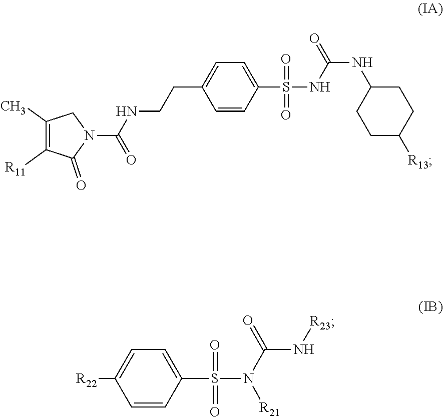

The present invention also provides a method for preparing the compound of formula IA, which is one of the following two methods:

### Method 1:

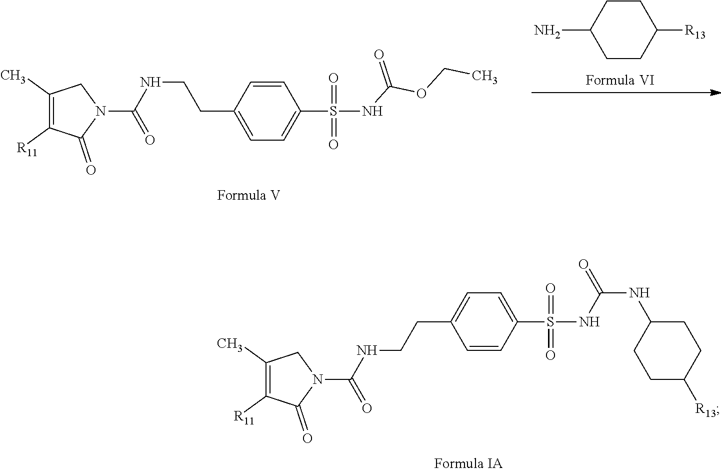

### Method 2:

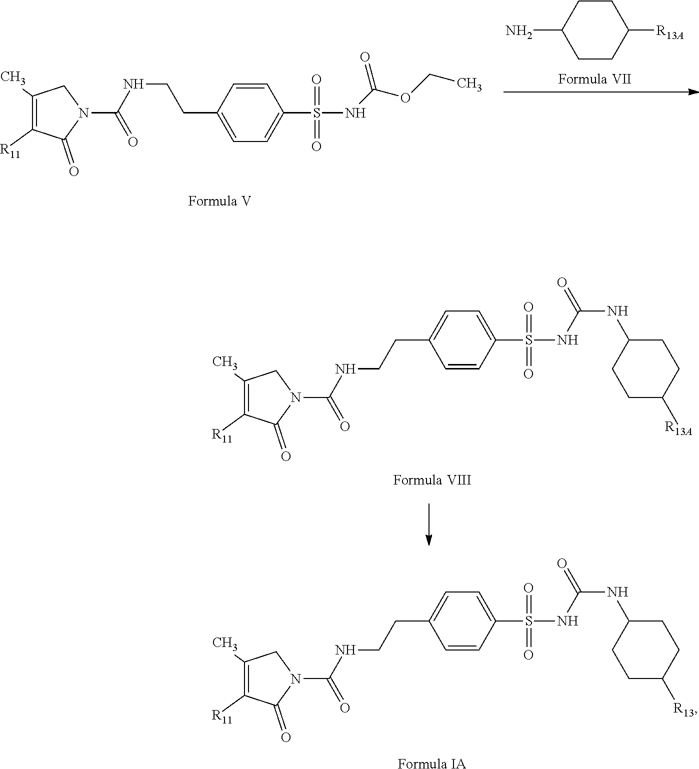

The present invention also provides a method for preparing the compound of formula IB, as follows:

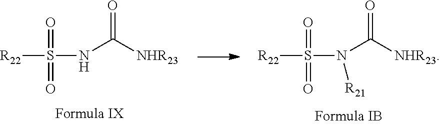

The present invention also provides a pharmaceutical composition comprising one or more selected from the group consisting of the aforementioned sulfonylurea compound and the stereoisomer or pharmaceutically acceptable salt thereof.

The present invention also provides a soluble epoxide hydrolase inhibitor, comprising one or more selected from the group consisting of the aforementioned sulfonylurea compound and the stereoisomer, pharmaceutically acceptable salt thereof, or comprising the aforementioned pharmaceutical composition.

The present invention also provides use of the aforementioned sulfonylurea compound, or the stereoisomer or pharmaceutically acceptable salt thereof, or the aforementioned pharmaceutical composition, wherein the use is selected from the group consisting of: use in preparation of a medicament for inhibiting the activity of soluble epoxide hydrolase; use in preparation of a medicament for increasing the level of epoxyeicosatrienoic acids (EETs), use in preparation of a medicament for reducing inflammatory response, and use in preparation of a medicament for preventing and/or treating heart failure.

The present invention also provides use of a sulfonylurea drug in preparation of a medicament for prevention and/or treatment of heart failure, wherein the sulfonylurea drug is selected from the group consisting of glimepiride, gliclazide, gliquidone, glibenclamide, glipizide, tolazamide, torasemide and acetohexamide.

The technical solution of the present invention has at least the following technical effects:

The compound of the present invention has soluble epoxide hydrolase (sEH) inhibitory activity, which can increase the level of epoxyeicosatrienoic acids (EETs), thereby reducing inflammatory response, and can be used for prevention and treatment of heart failure.

The preparation method of the present invention has the technical advantages of simple steps, high yield, and easily available raw materials.

### BRIEF DESCRIPTION OF DRAWINGS

FIG. 1 shows bar charts showing the inhibitory effect of compound I-1 on enzymic activity of sEH; wherein, * represents p<0.05 compared to the control group, #represents p<0.05 compared to the AUDA group.

FIG. 2 shows bar charts showing the inhibitory effect of compound I-4 on enzymic activity of sEH; wherein, * represents p<0.05 compared to the control group.

FIG. 3 shows bar charts showing the inhibitory effect of compound I-23 on enzymic activity of sEH; wherein, * represents p<0.05 compared to the control group.

FIG. 4 shows bar charts showing the inhibitory effect of glimepiride on enzymic activity of sEH; wherein, * represents p<0.05 compared to the control group, ** represents p<0.01 compared to the control group, *** represents p<0.001 compared to the control group, ##represents p<0.01 compared to the AUDA group, ###represents p<0.001 compared to the AUDA group.

FIG. 5 is a bar chart showing the inhibitory effect of compound I-1 on PE induced cardiomyocyte remodeling; wherein, * represents p<0.05 compared to the control group, #represents p<0.05 compared to the PE group, ##represents p<0.01 compared to the PE group.

FIG. 6 is a bar chart showing the inhibitory effect of compound I-4 on PE induced cardiomyocyte remodeling; wherein, * represents p<0.05 compared to the control group, #represents p<0.05 compared to the PE group.

FIG. 7 is a bar chart showing the inhibitory effect of compound I-23 on PE induced cardiomyocyte remodeling; wherein, * represents p<0.05 compared to the control group, #represents p<0.05 compared to the PE group, ##represents p<0.01 compared to the PE group.

FIG. 8 is a bar chart showing the inhibitory effect of glimepiride on PE induced cardiomyocyte remodeling; wherein,* Represents p<0.05, *** represents p<0.001.

FIG. 9 shows bar charts showing the inhibitory effect of compound I-1 on impaired cardiac function; wherein, * represents p<0.05 compared to the control group, ** represents p<0.01 compared to the control group, #represents p<0.05 compared to the TAC group, ##represents p<0.01 compared to the TAC group.

FIG. 10 shows HE staining images showing the inhibitory effect of compound I-1 on myocardial hypertrophy.

FIG. 11 shows Sirius red staining images showing the inhibitory effect of compound I-1 on myocardial fibrosis.

FIG. 12 is a bar chart showing the inhibitory effect of compound I-1 on the increase in BNP expression; wherein, * represents p<0.05 compared to the control group, ** represents p<0.01 compared to the control group, and ##represents p<0.01 compared to the TAC group.

FIG. 13 shows bar charts showing the inhibitory effect of compound I-23 on impaired cardiac function; wherein, * represents p<0.05 compared to the control group, ** represents p<0.01 compared to the control group, #represents p<0.05 compared to the TAC group, and ##represents p<0.01 compared to the TAC group.

FIG. 14 shows HE staining images showing the inhibitory effect of compound I-23 on myocardial hypertrophy.

FIG. 15 shows Sirius red staining images showing the inhibitory effect of compound I-23 on myocardial fibrosis.

FIG. 16 shows bar charts showing the inhibitory effect of glimepiride on the increase in enzymic activity of sEH in the hearts of mice with chronic heart failure; wherein, && represents p<0.01 compared to the control group, * represents p<0.05 compared to the TAC group, and ** represents p<0.01 compared to the TAC group.

FIG. 17 shows bar charts showing the inhibitory effect of glimepiride on impaired cardiac function in the hearts of mice with chronic heart failure; wherein, * represents p<0.05, ** represents p<0.01, *** represents p<0.001.

FIG. 18 shows HE staining images showing the inhibitory effect of glimepiride on myocardial hypertrophy in mice with chronic heart failure.

FIG. 19 is a statistical diagram showing the inhibitory effect of glimepiride on myocardial hypertrophy in mice with chronic heart failure; wherein, ** represents p<0.01.

FIG. 20 shows Sirius red staining images showing the inhibitory effect of glimepiride on the hearts of mice with chronic heart failure.

FIG. 21 shows Sirius red staining statistical graphs showing the inhibitory effect of glimepiride on the hearts of mice with chronic heart failure; wherein, * represents p<0.05, ** represents p<0.01, and *** represents p<0.001.

FIG. 22 shows photographs of hearts showing the reversal effect of compound I-23 and Empagliflozin on myocardial hypertrophy in mice with chronic heart failure.

FIG. 23 shows the heart weight/tibia length (HW/TL) showing the reversal effect of compound I-23 and Empagliflozin on myocardial hypertrophy in mice with chronic heart failure; wherein, *** represents p<0.001 compared to the control group, , #represents p<0.05 compared to the TAC group.

FIG. 24 shows bar charts showing the reversal effect of compound I-23 and Empagliflozin on impaired cardiac function in mice with chronic heart failure; wherein, *** represents p<0.001 compared to the control group; ###represents p<0.001 compared to the TAC group, #represents p<0.05 compared to the TAC group.

FIG. 25 shows HE staining images showing the reversal effect of compound I-23 and Empagliflozin on myocardial hypertrophy in mice with chronic heart failure.

FIG. 26 is a statistical graph showing the reversal effect of compound I-23 and Empagliflozin on myocardial hypertrophy in mice with chronic heart failure; wherein, *** represents p<0.001 compared to the control group, ###represents p<0.001 compared to the TAC group.

FIG. 27 shows Sirius red staining images showing the reversal effect of compound I-23 and Empagliflozin on myocardial fibrosis in mice with chronic heart failure.

FIG. 28 is a bar chart showing the inhibitory effects of compound I-23 and Empagliflozin on the increase in BNP expression; wherein, *** represents p<0.001 compared to the control group, #represents p<0.05 compared to the TAC group, ##represents p<0.01 compared to the TAC group.

### DETAILED DESCRIPTION OF THE INVENTION

In order to better illustrate the present invention, numerous specific details are provided in the following specific embodiments. It should be understood by those skilled in the art that the present invention can be implemented without certain specific details.

### Group Definition

The term “C₁-C₆ alkyl” refers to a straight or branched alkyl having 1 to 6 carbon atoms. For example, it includes, but is not limited to, methyl, ethyl, propyl, isopropyl, butyl, isobutyl, tert-butyl, amyl, isopentyl, hexyl, etc.

The term “C₃-C₇ cycloalkyl” refers to a cyclic alkyl having 3 to 7 carbon atoms, including, but not limited to, cyclopropyl, methylcyclopropyl, ethylcyclopropyl, dimethylcyclopropyl, cyclobutyl, methylcyclobutyl, ethylcyclobutyl, cyclopentyl, cyclohexyl, etc.

The term “C4-C8 N-heterocyclyl” refers to a saturated or unsaturated non-aromatic heterocyclyl having 4 to 8 carbon atoms and at least one nitrogen atom on the ring, including, but not limited to,

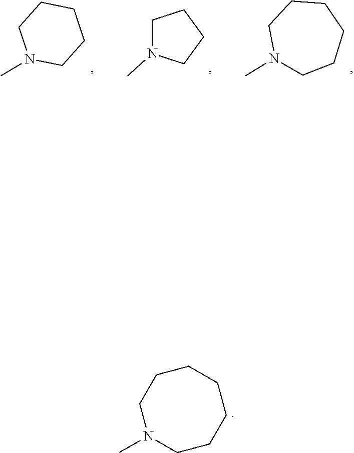

The term “C3-C6 cycloalkyl-fused pyrrolidinyl” includes, but is not limited to,

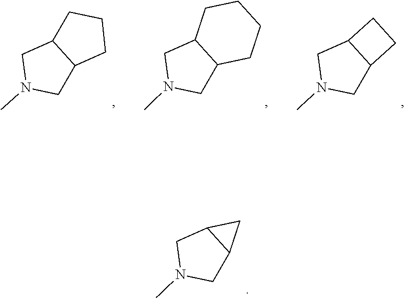

The term “C2-C6 alkenyl” refers to a straight or branched alkenyl having 2 to 6 carbon atoms, for example, including, but not limited to, propenyl, isopropenyl, butenyl, isobutenyl, tert-butenyl, pentenyl, isopentenyl, hexenyl, etc.

The term “C1-C2 alkyl substituted by C2-C6 alkenyl” refers to a C1-C2 alkyl substituted with the aforementioned “C2-C6 alkenyl”, for example, including, but not limited to, allyl, etc.

The term “C1-C6 acyl” refers to an acyl having 1 to 6 carbon atoms, for example, including, but not limited to, formyl, acetyl, propionyl, isopropyl, butyryl, isobutyryl, tert-butyryl, valeryl, isovaleryl, neovaleryl, hexyl, tert-hexyl, etc.

The term “C1-C6 alkoxy” refers to a straight, branched or cyclic alkoxy having 1 to 6 carbon atoms. For example, it includes, but is not limited to, methoxy, ethoxy, propioxy, isopropoxy, butoxy, isobutoxy, tert-butoxy, pentoxy, isopentoxy, cyclopentoxy, hexoxy, cyclohexoxy, etc.

The term “C1-C6 alkylene” refers to an alkylene having 1 to 6 carbon atoms, such as, but not limited to, methylene, methyl-substituted methylene, ethyl-substituted methylene, propyl-substituted methylene, ethylidene, methyl-substituted ethylene, ethyl-substituted ethylene, propylene, methyl-substituted propylene, ethyl-substituted propylene, pentene, hexylene, etc.

The term “aryl” refers to a substituent with aromatic ring structural properties, preferably is “C6-C14 aryl”, which represents an aryl having 6 to 14 carbon atoms, for example, including, but not limited to, phenyl, substituted phenyl, naphthyl, substituted naphthyl, anthryl, etc.

The term “heteroaryl” refers to a monocyclic or polycyclic group (each ring having 4 to 6 atoms) having 5 to 14 ring atoms, wherein one or more atoms are heteroatoms selected from N, O, or S, and the rest is carbon. The “heteroaryl” has certain aromatic properties. The preferred heteroaryl herein is “C2-C9 heteroaryl”, which represents a heteroaryl having 2 to 9 carbon atoms, including, such as, but not limited to, furyl, substituted furyl, benzofuranyl, substituted benzofuranyl, thienyl, substituted thienyl, benzothienyl, substituted benzothienyl, indolyl, substituted indolyl, isoindolyl, substituted isoindolyl, pyrrolyl, substituted pyrrolyl, thiazolyl, substituted thiazolyl, oxazolyl, substituted oxazolyl, pyrazolyl, substituted pyrazolyl, imidazolyl, substituted imidazolyl, pyranyl, substituted pyranyl, pyridazinyl, substituted pyridaziny, pyrazinyl, substituted pyrazinyl, pyrimidinyl, substituted pyrimidinyl, pyridyl, substituted pyridyl, quinolyl, substituted quinolyl, isoquinolyl, carbazolyl, substituted carbazolyl, etc.

The term “halogen” is selected from fluorine, chlorine, bromine, and iodine.

The term “halogenation” includes monohalogenation, polyhalogenation, or perhalogenation, that is, one or more, or all hydrogen atoms are replaced by halogen.

The term “substitution” means that one or more hydrogen atoms on a group are substituted by one or more substituents.

The term “5-6 membered lactam ring” refers to a saturated or unsaturated non-aromatic heterocyclic group having 5 to 6 ring atoms and containing an amide moiety on the ring, including, but not limited to,

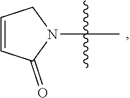 [SMILES: *N1CC=CC1=O]

where the hydrogen atom on the ring can be replaced by a substituent.

In one aspect, the present invention relates to a sulfonylurea compound of formula I, or a stereoisomer or pharmaceutically acceptable salt thereof:

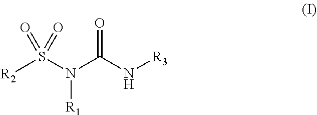

wherein,
R₁ is H,
R₂ is a C6-C14 aryl substituted with —(CH₂)₂NHCORₐ;
   Rₐ is a substituted 5-6 membered lactam ring, wherein the substituent for the “substituted” is one or more selected from C1-C6 alkyl;
R₃ is a substituted C3-C7 cycloalkyl; wherein the substituent for the “substituted” is selected from —C(═O)XR_{b} and —(CH₂)ₘOR_{c},
   X is —NH— or —O—;
   R_{b} is a C1-C6 alkyl, a C1-C6 acyl, or

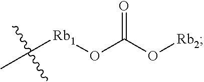

wherein Rei is a C1-C6 alkylene, R_{b2} is a C3-C7 cycloalkyl;
R_{c} is a C1-C6 alkyl, a halogenated C1-C6 alkyl, a C3-C7 cycloalkyl, a C3-C6 alkenyl, a C1-C6 acyl, —CO(CH₂)ₙNR_{d}Rₑ,

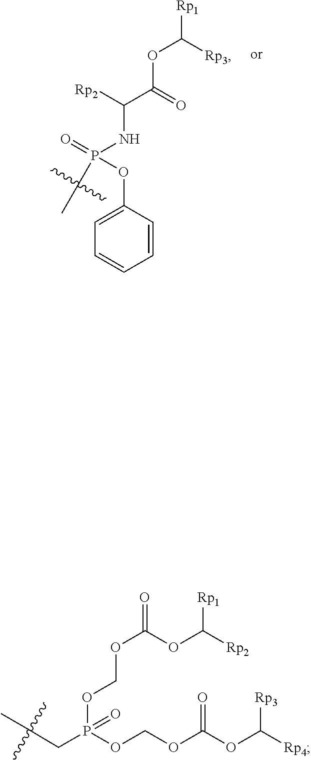

wherein R_{d} and Rₑ are each independently selected from H, C1-C6 alkyl, C3-C7 cycloalkyl, substituted or unsubstituted C6-C14 aryl; or R_{d} and Rₑ, together with the N atom connected thereto, form a substituted or unsubstituted pyrrolidinyl, piperidyl or piperazinyl; wherein the substituent for the “substituted” is selected from C1-C6 alkyl, halogenated C1-C6 alkyl, —OH, halogen, C1-C6 alkoxy, halogenated C1-C6 alkoxy and —CN;
Rₚ₁, Rₚ₂, Rₚ₃, and Rₚ₄ are each independently a C1-C6 alkyl; m and n are each independently an integer of 0 to 5, for example, 0, 1, 2, 3, 4, 5; or
R₁ is a C1-C6 alkyl, a C3-C6 cycloalkyl, and a C1-C2 alkyl substituted by C2-C6 alkenyl,
R₂ is a substituted C6-C14 aryl or a substituted 5-14 membered heteroaryl; wherein the substituent for the “substituted” is selected from C1-C6 alkyl, C1-C6 acyl, —(CH₂)_{q}R_{f}, —(CH₂)_{q}NHCOR_{g}; wherein q is an integer of 2 to 4;
R_{f} is a substituted isoquinolinedione group; R_{g} is a substituted C6-C14 aryl, a substituted 5-14 membered heteroaryl, or a substituted 5-6 membered lactam ring; wherein the substituent for the “substituted” is one or more selected from C1-C6 alkyl, halogenated C1-C6 alkyl, C1-C6 alkoxy, halogenated C1-C6 alkoxy, —OH, halogen, and —CN;
R₃ is a substituted or unsubstituted C1-C6 alkyl, a substituted or unsubstituted C3-C7 cycloalkyl, a substituted or unsubstituted 4-8 membered N-heterocyclyl, a substituted or unsubstituted C3-C6 cycloalkyl-fused pyrrolidinyl, or

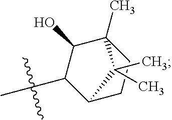

wherein the substituent for the “substituted” is selected from C1-C4 alkyl, —C(═O)XRₕ, and —(CH₂)ₘORᵢ;
X is NH or O;
Rₕ is H, a C1-C6 alkyl, a C1-C6 acyl or

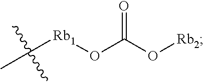

wherein R_{b1} is a C1-C6 alkylene; R_{b2} is a C3-C7 cycloalkyl;
Rᵢ is H, a C1-C6 alkyl, a halogenated C1-C6 alkyl, a C3-C7 cycloalkyl, a C3-C6 alkenyl, a C1-C6 acyl, —CO(CH₂)ₙNR_{d}Rₑ,

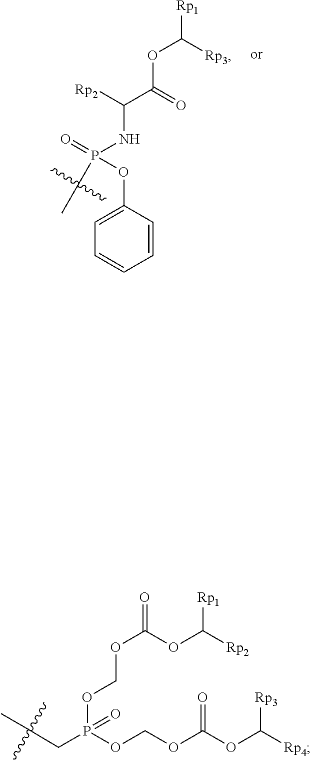

wherein Rₚ₁, Rₚ₂, Rₚ₃ and Rₚ₄ are each independently a C1-C6 alkyl;
R_{d} and Rₑ are each independently selected from H, C1-C6 alkyl, C3-C7 cycloalkyl, and substituted or unsubstituted C6-C14 aryl; or R_{d} and Rₑ, together with the N atom connected thereto, form a substituted or unsubstituted pyrrolidinyl, piperidyl or piperazinyl; wherein the substituent for the “substituted” is selected from C1-C6 alkyl, halogenated C1-C6 alkyl, —OH, halogen, C1-C6 alkoxy, halogenated C1-C6 alkoxy and —CN;
Rₚ₁, Rₚ₂, Rₚ₃, and Rₚ₄ are each independently a C1-C6 alkyl;
m and n are each independently an integer of 0 to 5, for example, 0, 1, 2, 3, 4, 5.

The sulfonylurea compound of the present invention can be used as a sEH inhibitor with strong inhibitory activity on sEH enzymes, and some compounds have an IC₅₀ lower than 1 μM.

In one embodiment, the sulfonylurea compound of formula I of the present invention may be selected from the compounds of formulas IA and IB below.

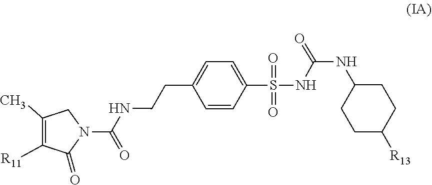

wherein,
R₁₁ is a C1-C6 alkyl;
R₁₃ is —C(═O)XR_{b} or —(CH₂)ₘOR_{c};
X, R_{b}, R_{c} and m are defined as above.

In some embodiments, R_{b} is a C1-C3 alkyl, a C1-C3 acyl, or

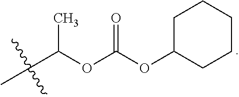 [SMILES: *C(C)OC(=O)OC1CCCCC1]

In some embodiments, R_{c} is a C1-C3 alkyl, a C3-C6 cycloalkyl, a C1-C3 acyl, —CO(CH₂)ₙNR_{d}Rₑ,

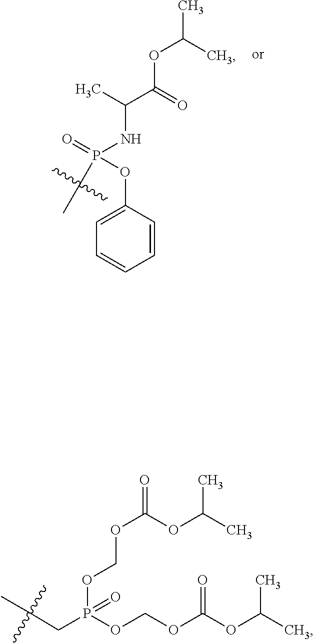

wherein R_{d}, Rₑ and n are defined as above.

In some embodiments, R_{d} and Rₑ are each independently selected from H, C1-C3 alkyl, C3-C6 cycloalkyl, and substituted or unsubstituted phenyl, or R_{d} and Rₑ, together with the N atom connected thereto, form a substituted or unsubstituted pyrrolidinyl, piperidyl or piperazinyl; wherein the substituent for the “substituted” is selected from C1-C3 alkyl, halogenated C1-C3 alkyl, —OH, halogen, C1-C3 alkoxy, halogenated C1-C3 alkoxy and —CN.

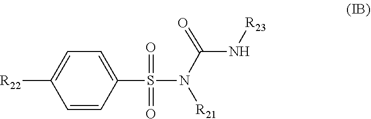

wherein,
R₂₁ is a C1-C6 alkyl, C3-C6 cycloalkyl, or a C1-C2 alkyl substituted by C2-C6 alkenyl;
R₂₂ is a C1-C6 alkyl, a C1-C6 acyl, —(CH₂)_{q}R_{f} or —(CH₂)_{q}NHCOR_{g}, wherein, q is an integer of 2 to 4;
R₂₃ is a C1-C6 alkyl, a C3-C6 cycloalkyl, a 4-8 membered N-heterocyclyl, a cyclopentyl-fused pyrrolidinyl,

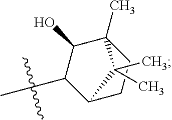

or a substituted cyclohexyl; wherein the substituent for the “substituted” is selected from C1-C4 alkyl, —C(═O)XRₕ, and —(CH₂)ₘORᵢ;
wherein, X, R_{f}, R_{g}, Rₕ and Rᵢ are defined as above.

In some embodiments, Rₕ is a C1-C3 alkyl, a C1-C3 acyl, or

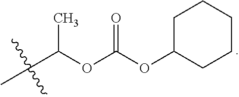 [SMILES: *C(C)OC(=O)OC1CCCCC1]

In some embodiments, Rᵢ is a C1-C3 alkyl, a C3-C6 cycloalkyl, a C1-C3 acyl, —CO(CH₂)ₙNR_{d}Rₑ,

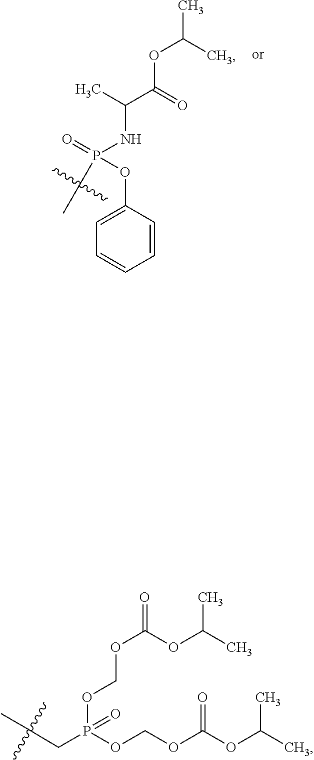

wherein R_{d}, Rₑ and n are defined as above.

In some embodiments, R_{d} and Rₑ are each independently selected from H, C1-C3 alkyl, C3-C6 cycloalkyl, and substituted or unsubstituted phenyl, or R_{d} and Rₑ, together with the N atom connected thereto, form a substituted or unsubstituted pyrrolidinyl, piperidyl or piperazinyl; wherein the substituent for the “substituted” is selected from C1-C3 alkyl, halogenated C1-C3 alkyl, —OH, halogen, C1-C3 alkoxy, halogenated C1-C3 alkoxy and —CN.

In some embodiments, the compound of formula IB is selected from the compounds of formulas IB1-IB7.

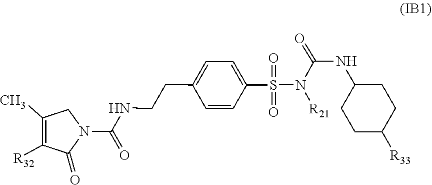

wherein,
R₂₁ is a C1-C6 alkyl, a C3-C6 cycloalkyl, or a C1-C2 alkyl substituted by C2-C6 alkenyl;
R₃₂ is a C1-C6 alkyl;
R₃₃ is a C1-C4 alkyl, —C(═O)XRₕ, or —(CH₂)ₘORᵢ;
X is NH or O;
Rₕ is H, a C1-C3 alkyl or

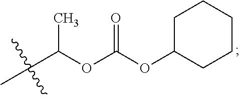 [SMILES: *C(C)OC(=O)OC1CCCCC1]

Rᵢ is H, a C1-C6 alkyl, a halogenated C1-C6 alkyl, a C3-C6 cycloalkyl, a C3-C6 alkenyl, a C1-C6 acyl, —CO(CH₂)ₙNR_{d}Rₑ,

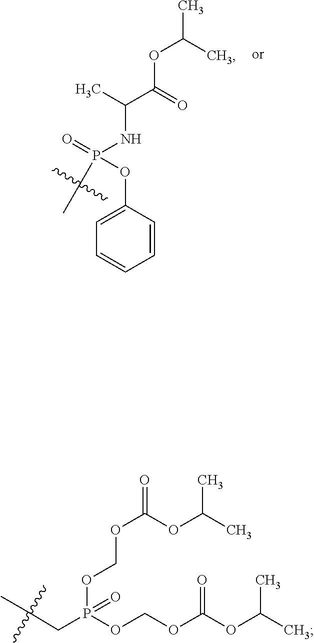

R_{d} and Rₑ are each independently selected from H, C1-C3 alkyl, C3-C6 cycloalkyl, and substituted or unsubstituted C6-C14 aryl; or R_{d} and Rₑ, together with the N atom connected thereto, form a substituted or unsubstituted pyrrolidinyl, piperidyl or piperazinyl; wherein the substituent for the “substituted” is selected from C1-C3 alkyl, halogenated C1-C3 alkyl, —OH, halogen, C1-C3 alkoxy, halogenated C1-C3 alkoxy and —CN;
m and n are each independently an integer of 0 to 5, for example, 0, 1, 2, 3, 4, 5.

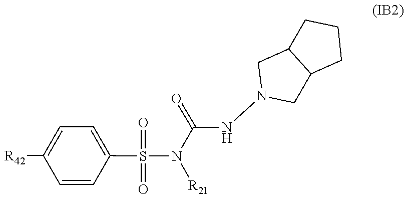

wherein,
R₂₁ is a C1-C6 alkyl, a C3-C6 cycloalkyl, or a C1-C2 alkyl substituted by C2-C6 alkenyl;
R₄₂ is a C1-C6 alkyl.

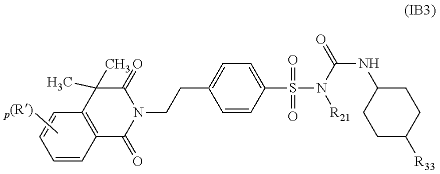

wherein,
R′ is each independently a C1-C6 alkyl or a C1-C6 alkoxy; p is an integer of 1 to 4, for example, 1, 2, 3, or 4;
R₂₁ is a C1-C6 alkyl, a C3-C6 cycloalkyl, or a C1-C2 alkyl substituted by C2-C6 alkenyl;
R₃₃ is H, a C1-C4 alkyl, —C(═O)XRₕ, or —(CH₂)ₘORᵢ;
X is NH or O;
Rₕ is H, a C1-C6 alkyl, or

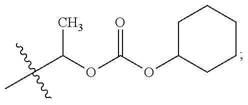 [SMILES: *C(C)OC(=O)OC1CCCCC1]

Rᵢ is H, a C1-C6 alkyl, a halogenated C1-C6 alkyl, a C3-C6 cycloalkyl, a C3-C6 alkenyl, a C1-C6 acyl, —CO(CH₂)ₙNR_{d}Rₑ,

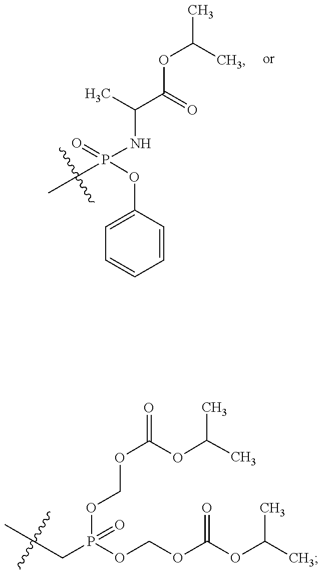

R_{d} and Rₑ are each independently selected from H, C1-C3 alkyl, C3-C6 cycloalkyl, and substituted or unsubstituted C6-C14 aryl, or R_{d} and Rₑ, together with the N atom connected thereto, form a substituted or unsubstituted pyrrolidinyl, piperidyl or piperazinyl; wherein the substituent for the “substituted” is selected from C1-C3 alkyl, halogenated C1-C3 alkyl, —OH, halogen, C1-C3 alkoxy, halogenated C1-C3 alkoxy and —CN;
m and n are each independently an integer of 0 to 5, for example, 0, 1, 2, 3, 4, 5.

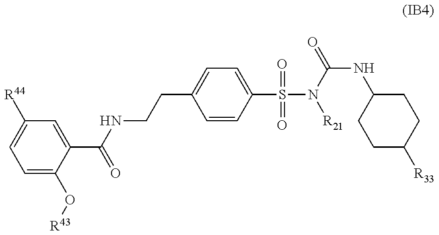

wherein,
R₂₁ is a C1-C6 alkyl, a C3-C6 cycloalkyl, or a C1-C2 alkyl substituted by C2-C6 alkenyl;
R₃₃ is H, a C1-C4 alkyl, —C(═O)XRₕ, or —(CH₂)ₘORᵢ;
X is NH or O;
Rₕ is H, C1-C3 alkyl, or

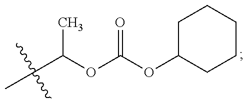 [SMILES: *C(C)OC(=O)OC1CCCCC1]

Rᵢ is H, a C1-C6 alkyl, a halogenated C1-C6 alkyl, a C3-C6 cycloalkyl, a C3-C6 alkenyl, a C1-C6 acyl, —CO(CH₂)ₙNR_{d}Rₑ,

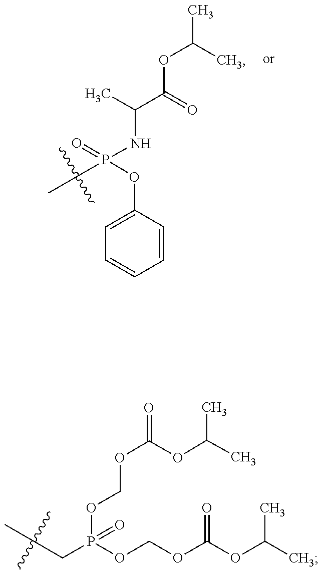

R_{d} and Rₑ are each independently selected from H, C1-C3 alkyl, C3-C6 cycloalkyl, and substituted or unsubstituted C6-C14 aryl; or R_{d} and Rₑ, together with the N atom connected thereto, form substituted or unsubstituted pyrrolidinyl, piperidyl or piperazinyl; the substituent for the “substituted” is selected from C1-C3 alkyl, halogenated C1-C3 alkyl, —OH, halogen, C1-C3 alkoxy, halogenated C1-C3 alkoxy and —CN;
m and n are each independently an integer of 0 to 5, for example, 0, 1, 2, 3, 4, 5;
R₄₃ is a C1-C6 alkyl;
R₄₄ is a halogen.

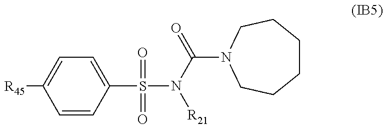

wherein,
R₂₁ is a C1-C6 alkyl, a C3-C6 cycloalkyl, or a C1-C2 alkyl substituted by C2-C6 alkenyl;
R₄₅ is a C1-C6 alkyl or —(CH₂)_{q}NHCOR_{g}; wherein R_{g} is a substituted 5-14 membered heteroaryl; q is an integer of 2-4; the preferred heteroaryl is isoxazolyl.

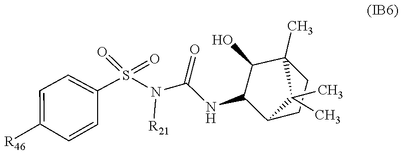

wherein,
R₂₁ is a C1-C6 alkyl, a C3-C6 cycloalkyl, or a C1-C2 alkyl substituted by C2-C6 alkenyl;
R₄₆ is a C1-C6 alkyl.

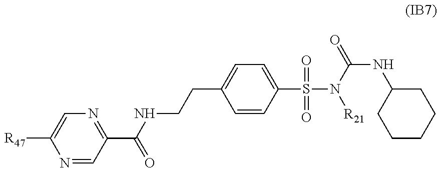

wherein,
R₂₁ is a C1-C6 alkyl, a C3-C6 cycloalkyl, or a C1-C2 alkyl substituted by C2-C6 alkenyl;
R₄₇ is a C1-C6 alkyl.

In some embodiments, the sulfonylurea compound of formula I of the present invention is selected from the following compounds:

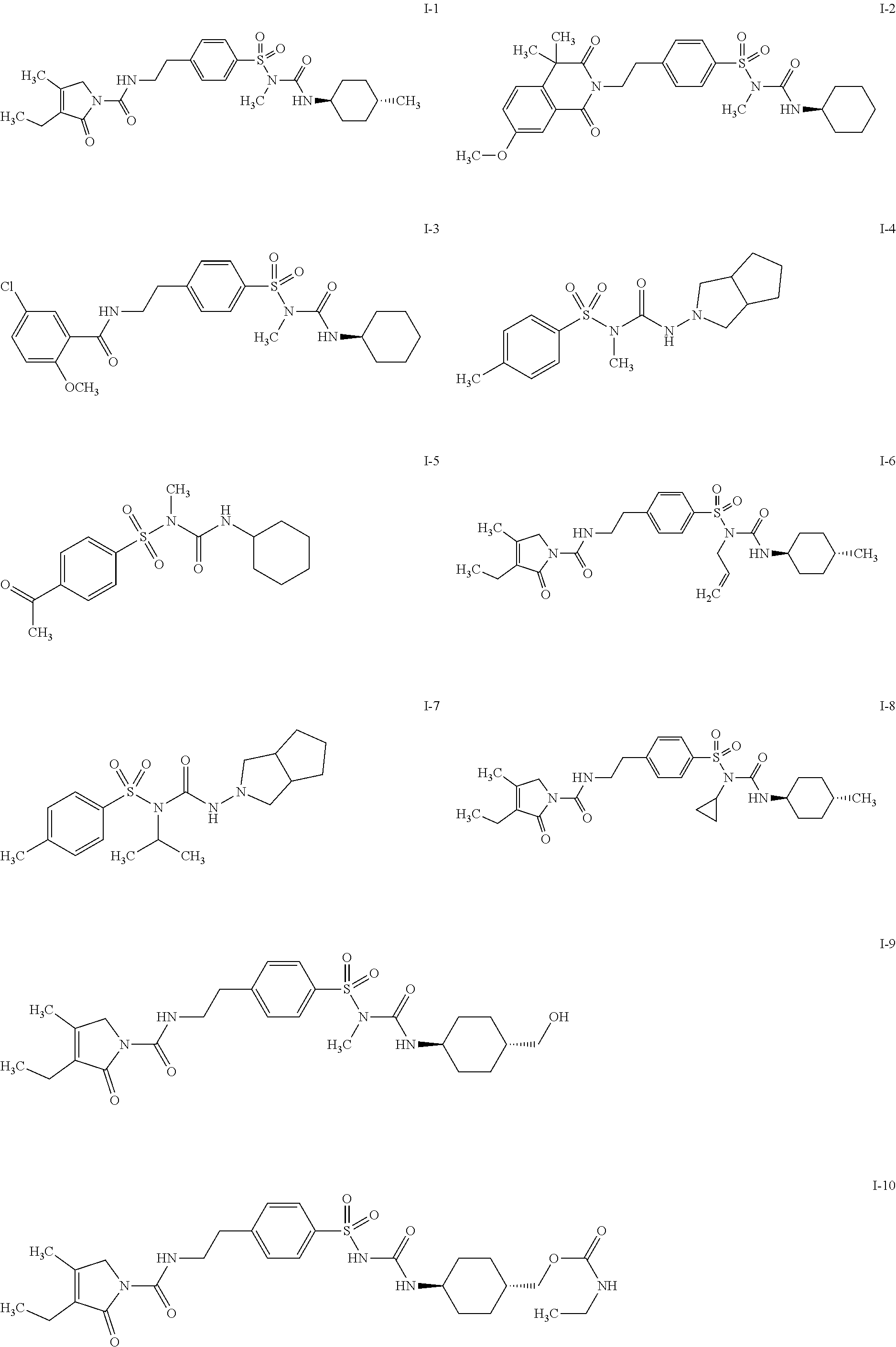

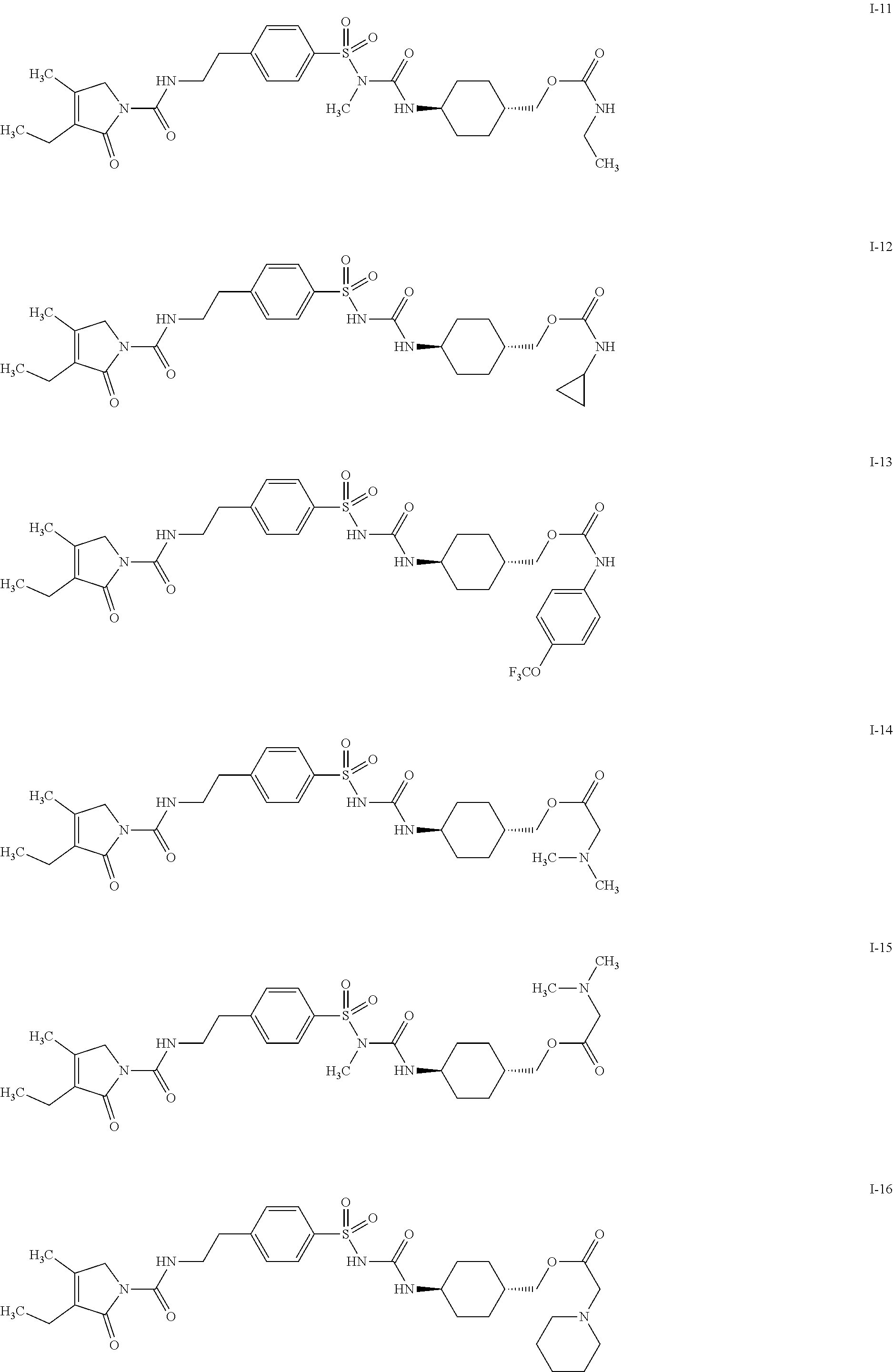

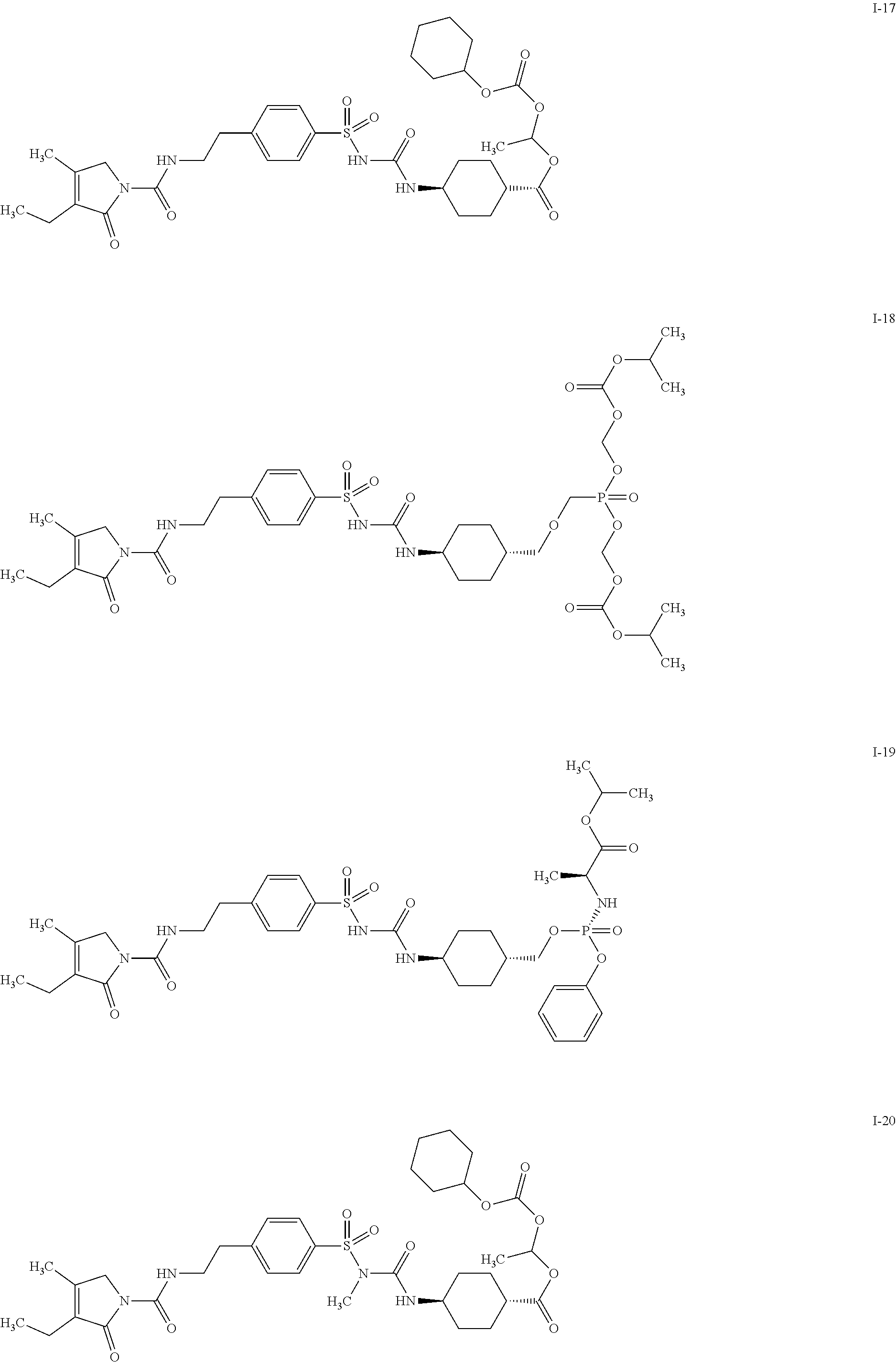

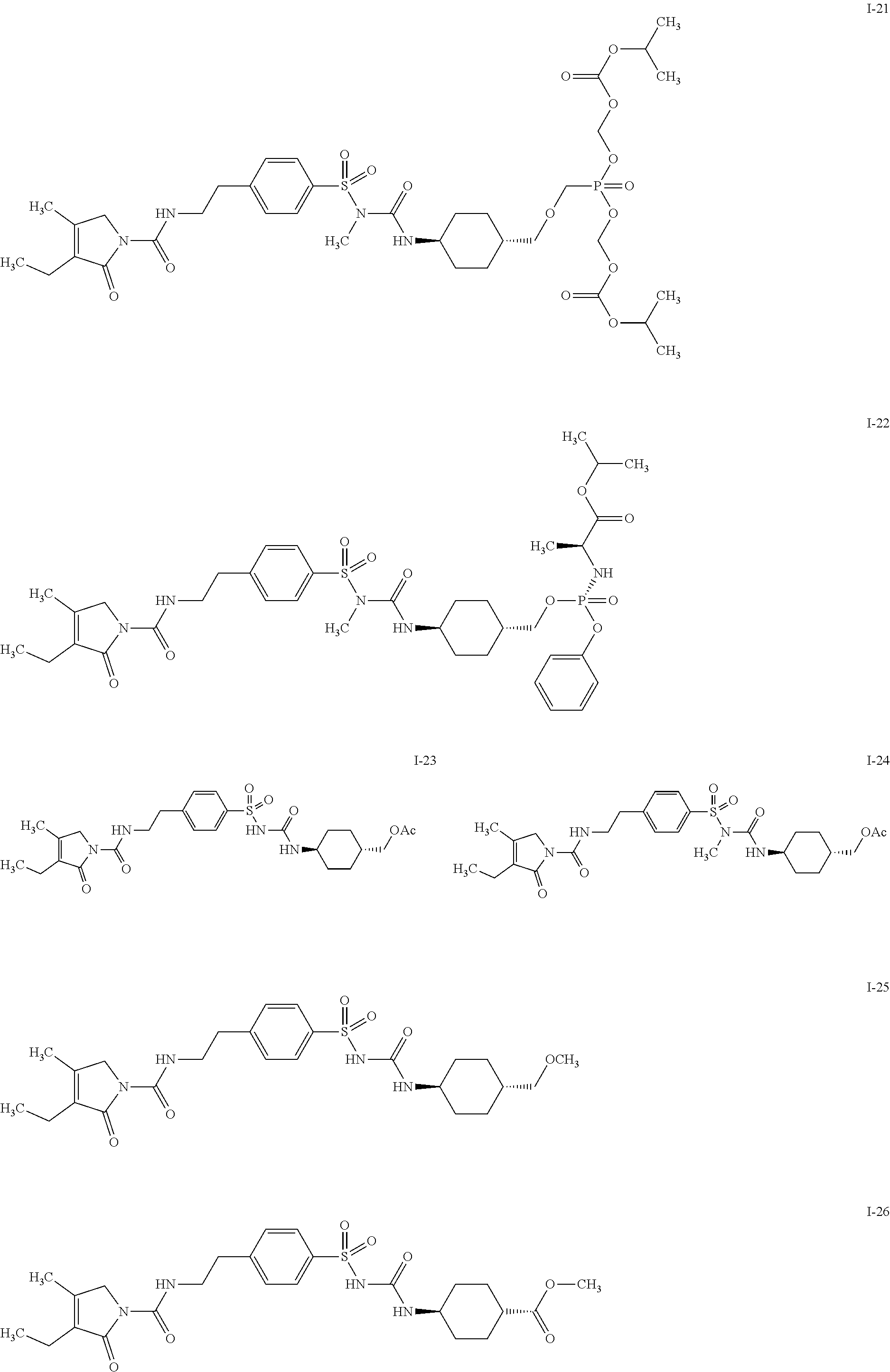

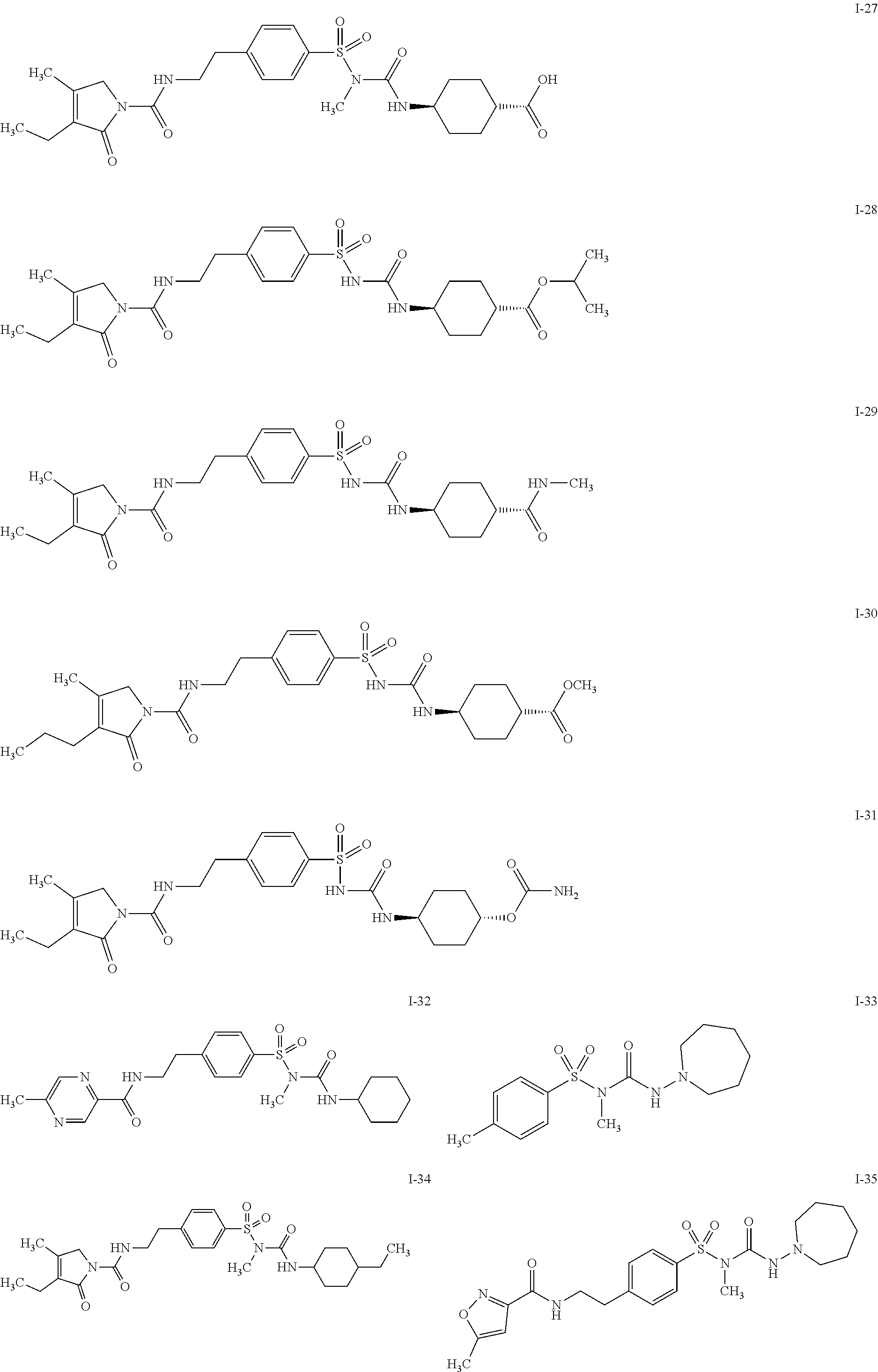

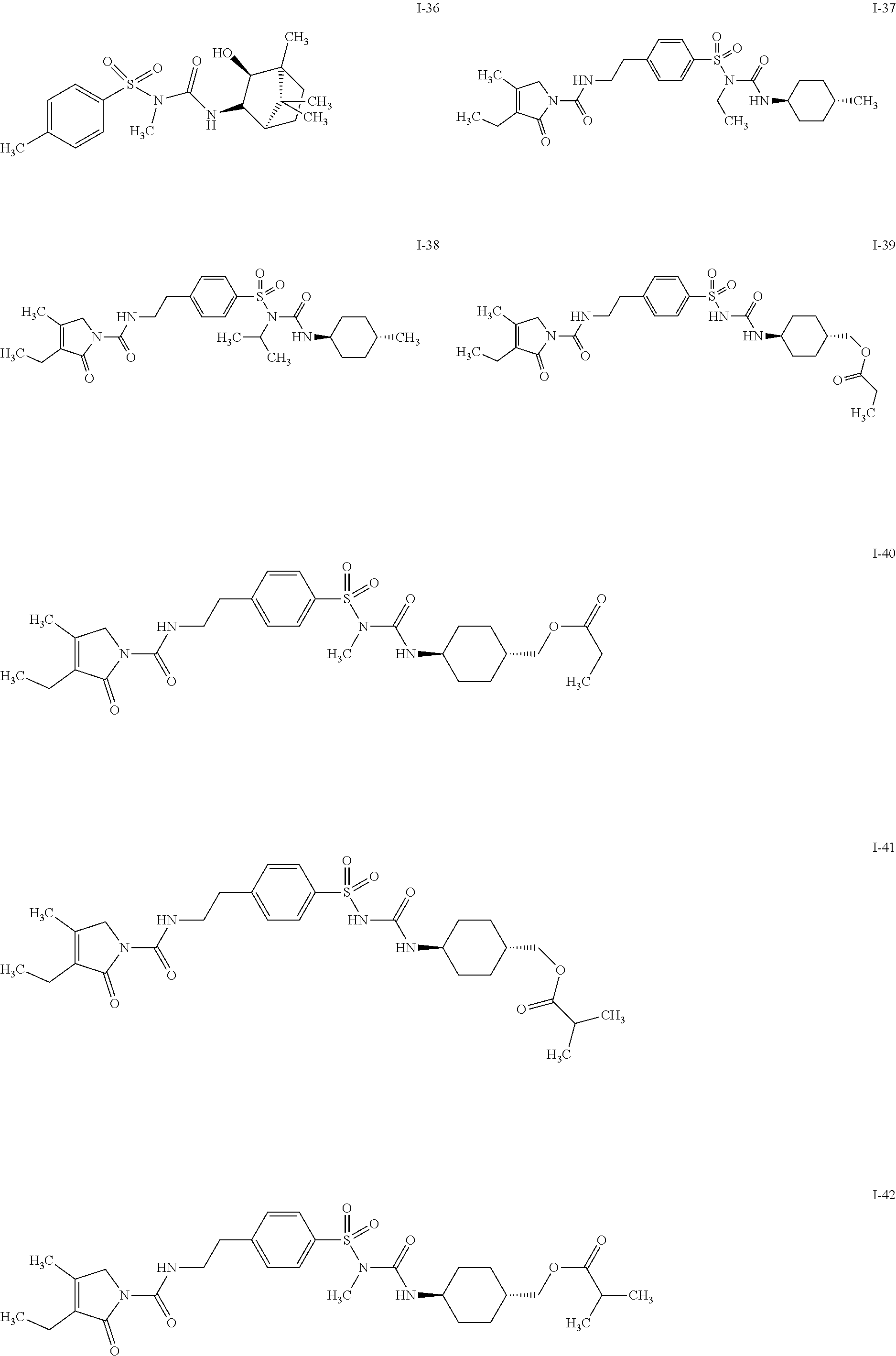

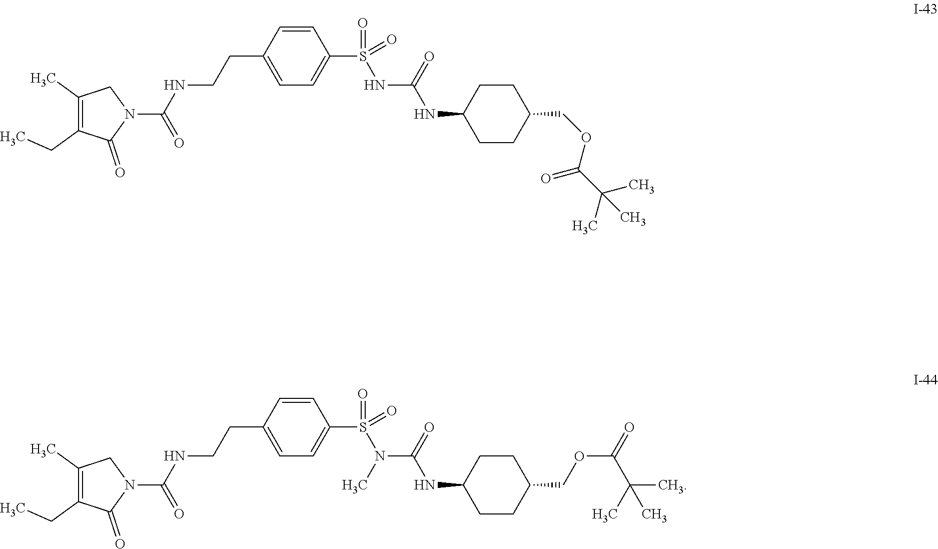

In some embodiments, the sulfonylurea compound, or the stereoisomer or pharmaceutically acceptable salt thereof of the present invention may exist in the form of a crystalline hydrate or solvate. These crystalline hydrate or solvate are also included within the scope of the present invention.

On the basis of knowing the structure of the compound of the present invention, those skilled in the art may design and synthesize the compound of the present invention using a reaction known in the art. Therefore, there is no special limitation on the specific preparation method for synthesizing the compound of the present invention, as long as the compound of the present invention can be obtained.

In some embodiments, the compound of formula IA of the present invention can be prepared using one of the following two methods:

### Method 1:

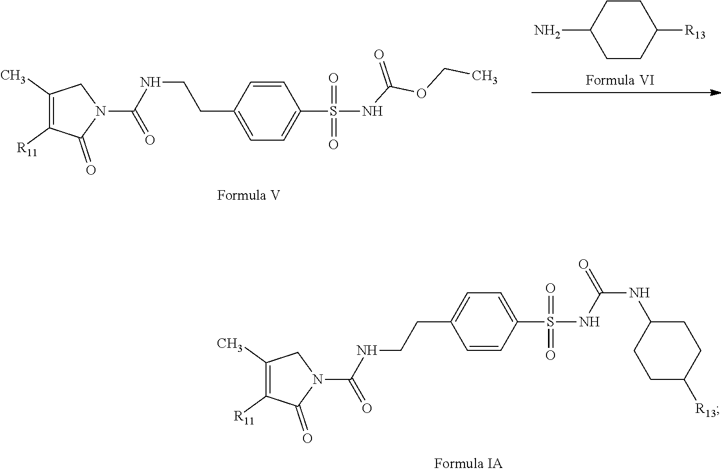

the compound of formula V reacts with the compound of formula VI through nucleophilic substitution to obtain the compound of formula IA;

### Method 2:

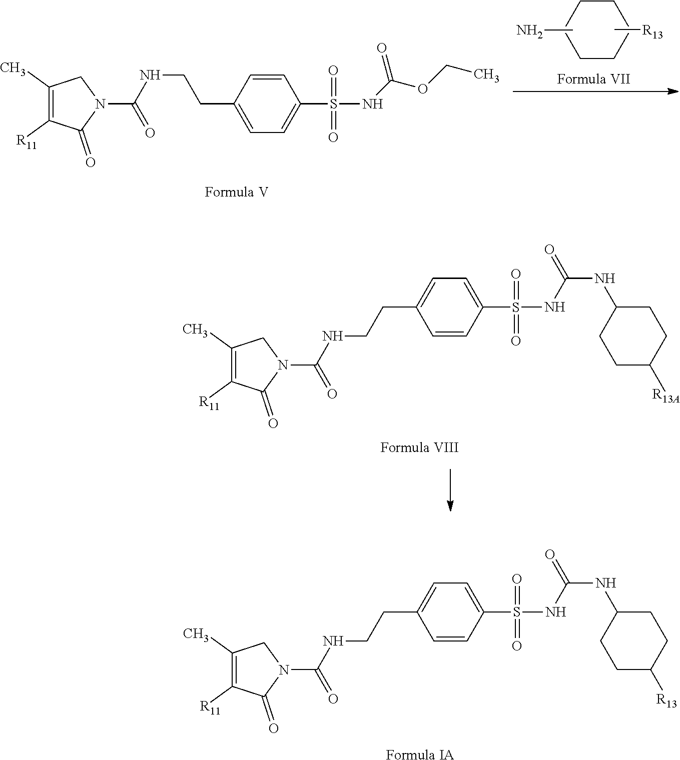

the compound of formula VII reacts with the compound of formula V through nucleophilic substitution to obtain the compound of formula VIII, which is then subjected to esterification, acylation, etherification, or phosphorylation to obtain the compound of Formula IA,

wherein, R_{13A} is —COOH or —(CH₂)ₘOH; and R₁₁, R₁₃ and m are defined as above.

In some embodiments, the compound of formula V is prepared by the method below:

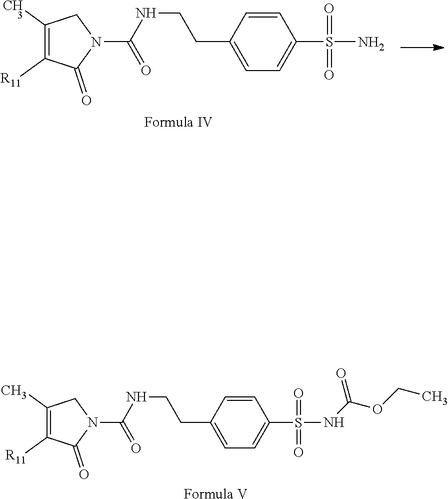

the compound of formula IV is subjected to acylation to prepare the sulfonylcarbamate of formula V;

wherein, R₁₁ is defined as above.

In some embodiments, the sulfonylcarbamate of formula V may be prepared by acylating the compound of formula IV with ethyl chloroformate in an organic solvent; wherein the preferred reaction temperature is the ice bath temperature, and the preferred organic solvent is dichloromethane.

In some embodiments, the compound of formula V and the compound of formula VI may be subjected to nucleophilic substitution in an organic solvent under protection of an inert gas (nitrogen or argon); wherein the preferred organic solvent is toluene, and the preferred reaction temperature is reflux temperature.

In some embodiments, the compound of formula IB of the present invention may be prepared by the following method:

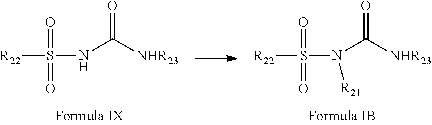

the compound of formula IX reacts with R₂₁—I through alkylation to obtain the compound of formula IB,

wherein, R₂₁, R₂₂ and R₂₃ are defined as above.

In some embodiments, the compound of formula IX is prepared by one of the following methods:

### Method A:

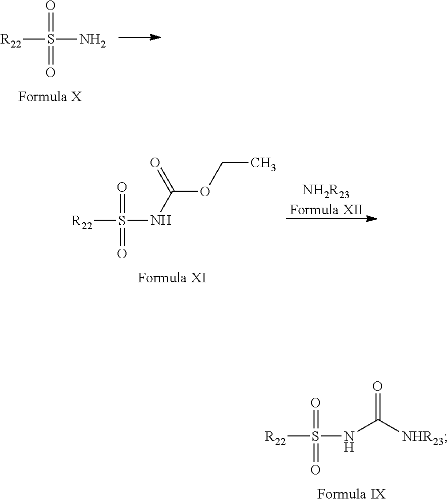

the compound of formula X is subjected to acylation to prepare the sulfonylcarbamate of formula XI, which then reacts with the compound of formula XII through nucleophilic substitution to obtain the compound of formula IX.

### Method B:

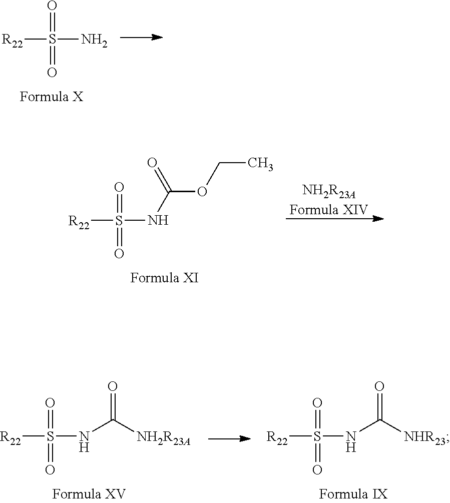

the compound of formula X is subjected to acylation to prepare the sulfonylcarbamate of formula XI, which then reacts with the compound of formula XIV through nucleophilic substitution to obtain the compound of formula XV; and the compound of formula XV is subjected to esterification, acylation, etherification, or phosphorylation to obtain the compound of formula IX;

wherein, R_{23A} is a substituted cyclohexyl, wherein the substituent for the “substituted” is selected from —COOH and —(CH₂)ₘOH; and R₂₂, R₂₃ and m are defined as above.

In some embodiments, the alkylation may be carried out using R₂₁—I and the compound of formula IX in the presence of potassium carbonate in an organic solvent as the solvent. R₂₁—I is added dropwise to an organic solvent dissolving with IX under an ice bath. After the dropwise addition is complete, the system is raised to room temperature. After the TLC detection indicates that the reaction is complete, water and ethyl acetate are added. After layered, the organic phase is washed with water, concentrated and purified by silica gel column chromatography. The preferred organic solvent is DMF.

In another respect, the present invention provides a pharmaceutical composition comprising one or more selected from the group consisting of the sulfonylurea compound and the stereoisomer and pharmaceutically acceptable salt thereof. The pharmaceutical composition may also include one or more pharmaceutically acceptable excipients, diluents, carriers, excipients, or adjuvants.

It has been experimentally confirmed that the compound of the present invention has inhibitory activity on soluble epoxide hydrolase (sEH), can increase the level of epoxyeicosatrienoic acids (EETs), and can thereby reduce inflammatory response and can be used for prevention and/or treatment of heart failure.

Therefore, in still another respect, the present invention further provides a soluble epoxide hydrolase inhibitor, comprising one or more selected from the group consisting of the sulfonylurea compound and the stereoisomer and pharmaceutically acceptable salt thereof, or comprising the pharmaceutical composition.

The present invention also provides a use of the sulfonylurea compound or the stereoisomer or pharmaceutically acceptable salt thereof, or the pharmaceutical composition, wherein the use is selected from the group consisting of: use in preparation of a medicament for inhibiting the activity of soluble epoxide hydrolase; use in preparation of a medicament for increasing the level of epoxyeicosatrienoic acids (EETs); use in preparation of a medicament for reducing inflammatory response; and use in preparation of a medicament for preventing and/or treating heart failure.

The present invention also provides the sulfonylurea compound or the stereoisomer or pharmaceutically acceptable salt thereof or the pharmaceutical composition for inhibition of the activity of soluble epoxide hydrolase, for increasing the level of epoxyeicosatetraenoic acids (EETs), for reducing inflammatory response, or for preventing and/or treating heart failure.

The present invention also provides a method for suppressing the activity of soluble epoxide hydrolase, a method for increasing the level of epoxyeicosatetraenoic acids (EETs), a method for reducing inflammatory response, or a method for preventing and/or treating heart failure, comprising: administering to a subject in need thereof an effective amount of the sulfonylurea compound or the stereoisomer or pharmaceutically acceptable salt thereof, or the pharmaceutical composition.

The present invention also provides use of a sulfonylurea drug in preparation of a medicament for prevention and/or treatment of heart failure, wherein the sulfonylurea drug is selected from the group consisting of glimepiride, gliclazide, gliquidone, glibenclamide, glipizide, tolazamide, torasemide and acetohexamide.

The present invention also provides a method for prevention and/or treatment of heart failure, comprising: administering to a subject in need thereof an effective amount of a sulfonylurea drug selected from the group consisting of glimepiride, gliclazide, gliquidone, glibenclamide, glipizide, tolazamide, torasemide and acetohexamide.

| Sulfonylurea drug | Structure |
|---|---|
| glimepiride | 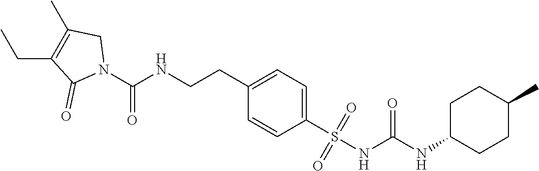 [SMILES: CCC1=C(C)CN(C(=O)NCCc2ccc(S(=O)(=O)NC(=O)N[C@H]3CC[C@H](C)CC3)cc2)C1=O] |
| gliclazide | 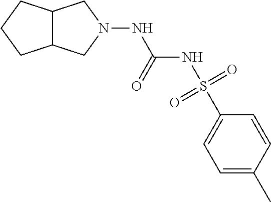 [SMILES: Cc1ccc(S(=O)(=O)NC(=O)NN2CC3CCCC3C2)cc1] |
| gliquidone | 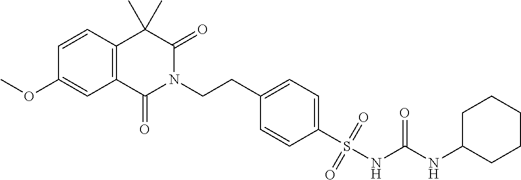 [SMILES: COc1ccc2c(c1)C(=O)N(CCc1ccc(S(=O)(=O)NC(=O)NC3CCCCC3)cc1)C(=O)C2(C)C] |
| glibenclamide | 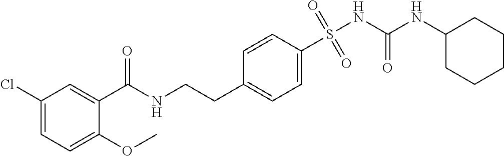 [SMILES: COc1ccc(Cl)cc1C(=O)NCCc1ccc(S(=O)(=O)NC(=O)NC2CCCCC2)cc1] |
| glipizide | 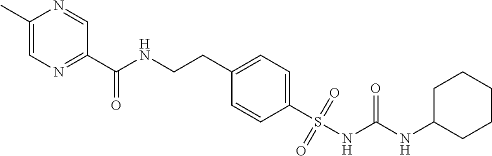 [SMILES: Cc1cnc(C(=O)NCCc2ccc(S(=O)(=O)NC(=O)NC3CCCCC3)cc2)cn1] |
| tolazamide | 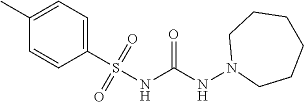 [SMILES: Cc1ccc(S(=O)(=O)NC(=O)NN2CCCCCC2)cc1] |
| torasemide | 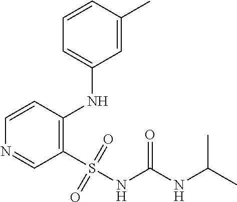 [SMILES: Cc1cccc(Nc2ccncc2S(=O)(=O)NC(=O)NC(C)C)c1] |
| acetohexamide | 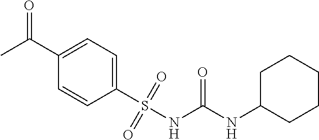 [SMILES: CC(=O)c1ccc(S(=O)(=O)NC(=O)NC2CCCCC2)cc1] |

### EXAMPLE

In order to clarify the purpose, technical solution, and advantages of the examples of the present invention, the following will provide a clear and complete description of the technical solution in the examples of the present invention. Obviously, the described examples are partial examples of the present invention, rather than the entire examples. Based on the examples in the present invention, all other examples, which are obtained by those skilled in the art without creative labor, fall within the scope of protection of the present invention. In some examples, the raw materials, components, methods, means, etc. familiar to those skilled in the art are not described in detail to highlight the main idea of the present invention.

The following examples further explain the synthesis method of the compounds and intermediates of the present invention, without limiting the scope of the present invention.

¹H NMR was performed on Bruker-400 or Bruker-500. Low-resolution mass spectrometry was performed on a Thermo Fisher FINNIGAN LTQ mass spectrometer. The reagents and raw materials used in the present invention are all commercially available raw materials unless otherwise specified.

### Example 1

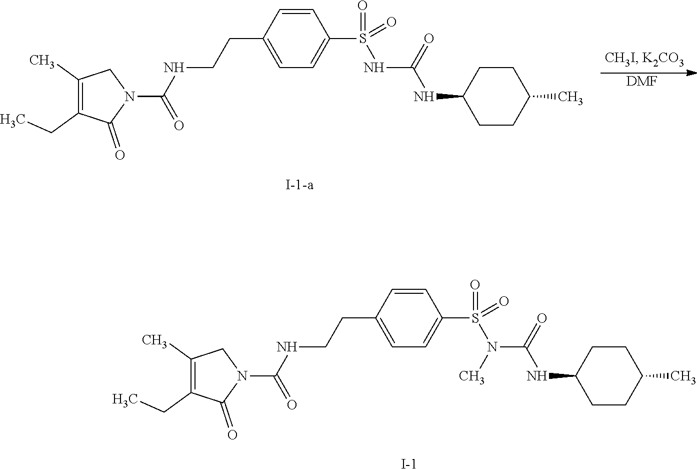

200 mg of I-1-a, 56 mg of potassium carbonate and 2 ml of N,N-dimethylformamide (DMF) were added into a 25 ml of three necked flask, and 55 mg of methyl iodide was dropped into the reaction solution at 0° C. The reaction solution was warmed to 25° C. and reacted for 1 h. 2 ml of water and 10 ml of ethyl acetate were added to the reaction solution and layered. The organic phase was washed three times with water (2 ml×3), concentrated and purified by column chromatography to obtain 105 mg of I-1 as a white solid with a yield of 51.0%.¹H NMR (DMSO-d₆, 500 MHz): 8.37 (t, 1H), 7.79 (d, 2H), 7.50 (d, 2H), 7.23 (d, 1H), 4.17 (s, 2H), 3.51 (q, 2H), 3.05 (s, 3H), 2.92 (t, 2H), 2.19 (q, 2H), 2.02 (s, 3H), 1.73 (d, 2H), 1.64 (d, 2H), 1.28 (m, 2H), 0.98 (t, 3H), 0.92 (m, 2H), 0.85 (d, 3H). ESI-MS m/z 505.3 (M+H)⁺.

### Example 2

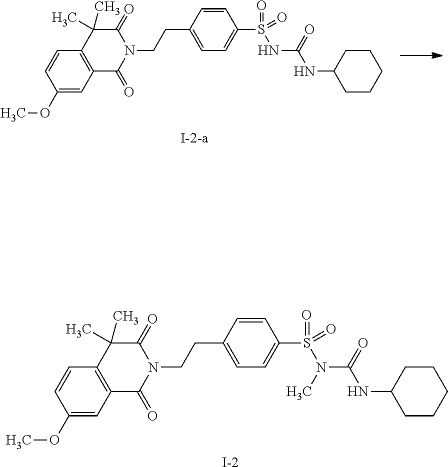

I-2 was prepared with a yield of 65.3% following the same method as that in Example 1, except that I-1-a was replaced with I-2-a. ¹H NMR (CDCl₃, 500 MHz): 7.75 (m, 2H), 7.69 (d, 1H), 7.49 (m, 2H), 7.37 (d, 1H), 7.22 (m, 2H), 4.27 (m, 2H), 3.91 (s, 3H), 3.12 (s, 3H), 3.05 (m, 2H), 1.91 (m, 1H), 1.74 (dt, 2H), 1.65 (s, 3H), 1.63 (m, 1H), 1.55 (s, 6H), 1.42-1.24 (m, 6H). ESI-MS m/z 542.2 (M+H)⁺.

### Example 3

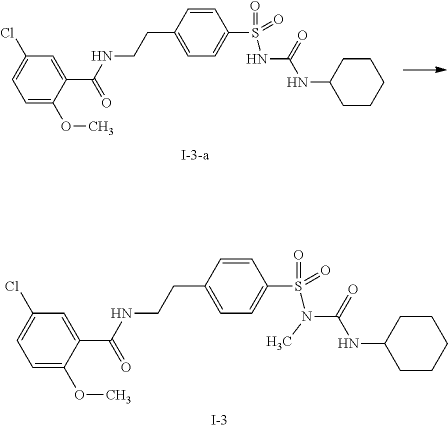

I-3 was prepared with a yield of 72.3% following the same method as that in Example 1, except that I-1-a was replaced with I-3-a. ¹H NMR (CDCl₃, 500 MHz): 8.19 (d, 1H), 7.83 (s, 1H), 7.79 (d, 2H), 7.45 (d, 2H), 7.41 (dd, 1H), 7.23 (d, 1H), 6.90 (d, 1H), 3.81 (s, 3H), 3.77 (q, 2H), 3.14 (s, 3H), 3.05 (t, 2H), 1.78 (m, 3H), 1.30 (m, 6H). ESI-MS m/z 508.2 (M+H)⁺.

### Example 4

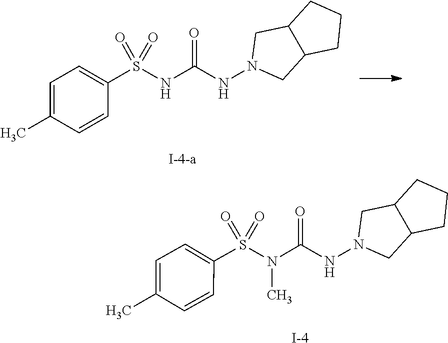

I-4 was prepared with a yield of 61.6% following the same method as that in Example 1, except that I-1-a was replaced with I-4-a. ¹H NMR (CDCl₃, 500 MHz): 7.96 (s, 1H), 7.72 (d, 2H), 7.36 (d, 2H), 3.23 (d, 2H), 3.13 (s, 2H), 2.66 (d, 2H), 2.46 (m, 5H), 1.68-1.50 (m, 6H).

ESI-MS m/z 338.4 (M+H)⁺.

### Example 5

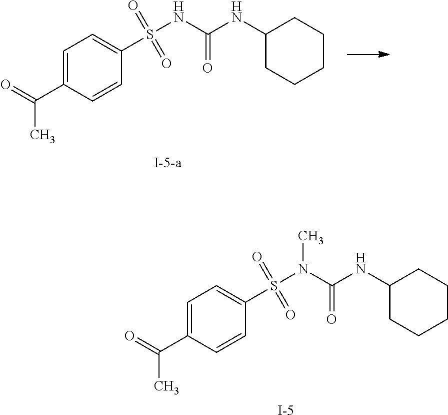

I-5 was prepared with a yield of 58% following the same method as that in Example 1, except that I-1-a was replaced with I-5-a. ESI-MS m/z 339.1 (M+H)⁺.

### Example 6

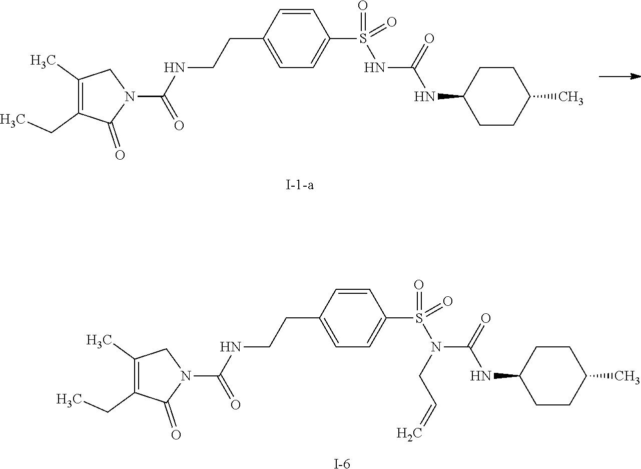

I-6 was prepared with a yield of 43% following the same method as that in Example 1, except that iodomethane was replaced with allyl bromide. ESI-MS m/z 531.3 (M+H)⁺.

### Example 7

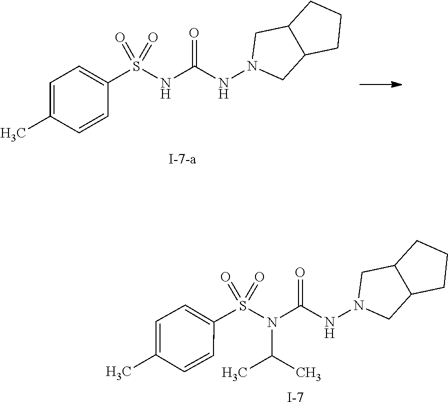

I-7 was prepared with a yield of 53% following the same method as that in Example 1, except that I-1-a was replaced with I-7-a and iodomethane was replaced with isopropyl bromide. ESI-MS m/z 366.2 (M+H)⁺.

### Example 8

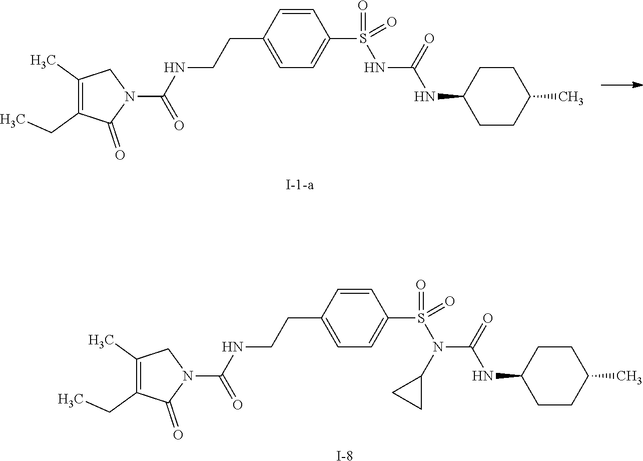

I-8 was prepared with a yield of 52% following the same method as that in Example 1, except that iodomethane was replaced with cyclopropyl bromide. ESI-MS m/z 531.2 (M+H)⁺.

### Example 9

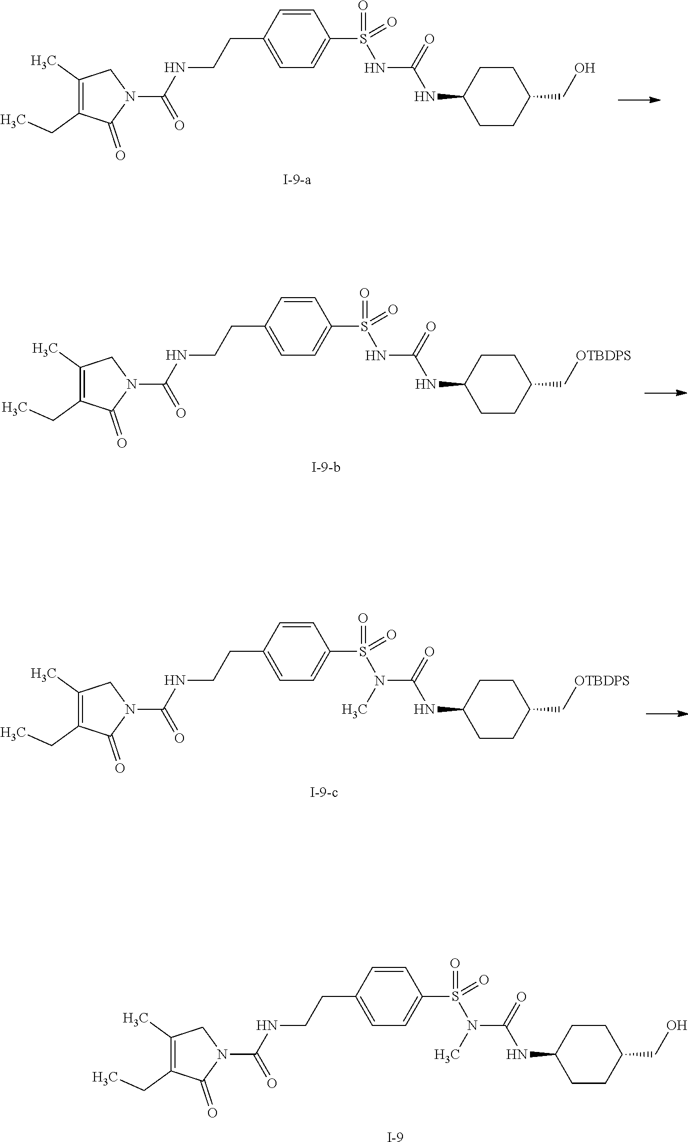

I-9-a (1.1 g, 2.18 mmol) was dissolved in 20 ml of DMF, and imidazole (0.75 g, 10.92 mmol) was added. tert-Butylchlorodiphenylsilane (TBDPSCl) (0.6 ml, 2.29 mmol) was dropped therein, and stirred overnight. TLC showed that the raw material was completely converted, and the reaction mixture was diluted with ethyl acetate. The organic phase was washed with water and saline, dried with anhydrous sodium sulfate, filtered and concentrated to obtain I-9-b, which was directly used for the next step without further purification.

I-9-c was prepared following the same method as that in Example 1, except that I-1-a was replaced with I-9-b.

I-9-c (0.2 g, 0.26 mmol) was dissolved in tetrahydrofuran, added dropwisely with 1.5 mL of concentrated hydrochloric acid, and stirred at room temperature for 5 hours. TLC showed that the raw material was completely converted, and the reaction mixture was diluted with dichloromethane. The organic phase was wash with saline, dried with anhydrous sodium sulfate, filtered, concentrated and purified to obtain I-9 with a yield of 54%. ESI-MS m/z 521.2 (M+H)⁺.

### Example 10

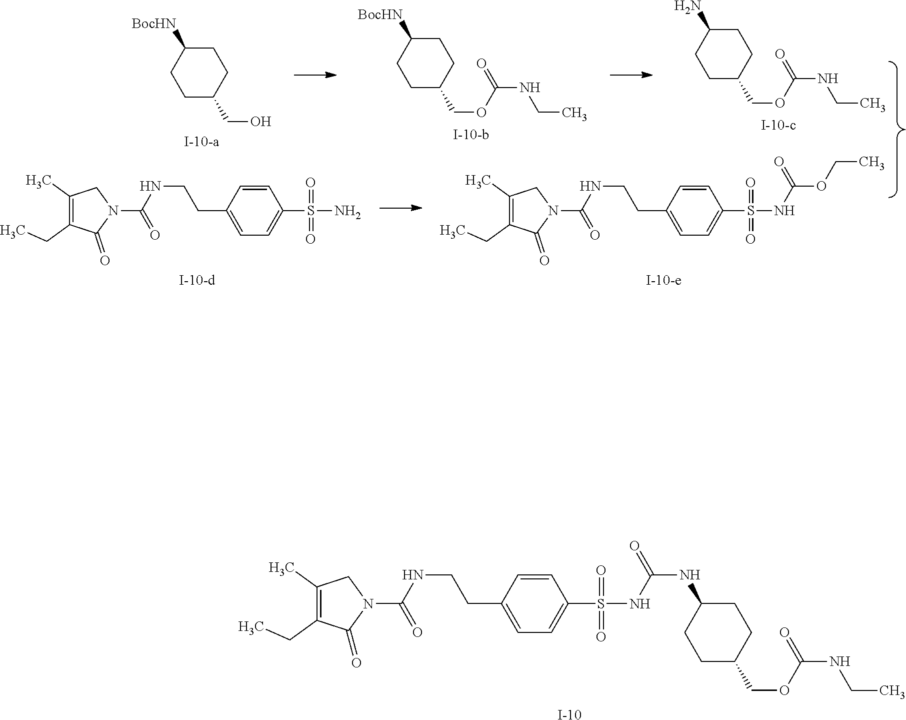

Step 1: I-10-a (143 mg, 0.62 mmol) was suspended in dichloromethane (DCM), added with 3 eq of triethylamine, followed by dropwise addition of 2.5 eq of ethyl isocyanate at room temperature. The reaction mixture was reacted overnight at room temperature (10-18° C.), and TLC showed that the raw material was completely converted. The reaction mixture was diluted with ethyl acetate (EA). The organic phase was washed with diluted hydrochloric acid (14 mL of 1N hydrochloric acid was diluted to 80 mL) and saline, dried, filtered and concentrated to obtain I-10-b (180 mg, 0.6 mmol) as a white solid.

Step 2: I-10-b (180 mg, 0.6 mmol) was dissolved in DCM, added dropwisely with 10 eq of trifluoroacetic acid at room temperature, and stirred at room temperature for 3 hours after adding. The reaction mixture was concentrated to remove most of the trifluoroacetic acid, and the residue was diluted with DCM and then added with aqueous sodium bicarbonate to adjust the pH to be weakly basic. The organic phase was separated, and the aqueous phase was repeatedly extracted with DCM. The combined organic phase was dried, filtered and concentrated to obtain I-10-c.

Step 3: I-10-d (2 g, 5.70 mmol) was suspended in 30 mL of dichloromethane, and added with triethylamine (1.98 mL, 14.2 mmol), followed by dropwise addition of ethyl chloroformate (810 μL, 8.54 mmol). After the addition, the temperature was slowly raised to room temperature. After 7 hours, TLC showed that the raw material was completely converted. The reaction mixture was diluted with 100 mL of dichloromethane, and the organic phase was washed with diluted hydrochloric acid (14 mL of 1N hydrochloric acid was diluted to 80 mL) and saline, dried with anhydrous sodium sulfate, concentrated to obtain a crude, which was added with 8 mL of acetone, stirred for 2 hours under reflux and then for 2 hours at room temperature, filtered, dried to obtain I-10-e as a white solid.

Step 4: I-10-c (50 mg, 0.25 mmol) and I-10-e (105 mg, 0.25 mmol) were mixed in a small amount of toluene, purged with nitrogen, and refluxed for 5 hours. The reaction mixture was concentrated, and slurred in acetone to obtain a total of 95 mg of I-10. ¹H NMR (400 MHz, DMSO-d₆) δ 10.35 (s, 1H), 8.37 (t, J=5.8 Hz, 1H), 7.87-7.73 (m, 2H), 7.46 (d, J=8.2 Hz, 2H), 7.02 (d, J=6.1 Hz, 1H), 6.31 (d, J=7.7 Hz, 1H), 4.17 (s, 2H), 3.71 (d, J=6.5 Hz, 2H), 3.50 (q, J=6.8 Hz, 2H), 3.19 (dd, J=7.7, 3.8 Hz, OH), 2.97 (qd, J=7.2, 5.6 Hz, 2H), 2.90 (t, J=7.2 Hz, 2H), 2.18 (q, J=7.5 Hz, 2H), 2.01 (s, 3H), 1.69 (dd, J=28.0, 12.3 Hz, 4H), 1.45 (s, 1H), 1.16-1.04 (m, 2H), 0.98 (td, J=7.4, 2.8 Hz, 7H). ESI-MS m/z 578.3 (M+H)⁺.

### Example 11

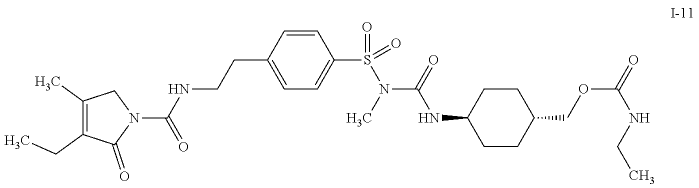 [SMILES: CCNC(=O)OC[C@H]1CC[C@H](NC(=O)N(C)S(=O)(=O)c2ccc(CCNC(=O)N3CC(C)=C(CC)C3=O)cc2)CC1]

I-11 was prepared with a yield of 57% following the same method as that in Example 1, except that I-1-a was replaced with I-10. ESI-MS m/z 592.3 (M+H)⁺.

### Example 12

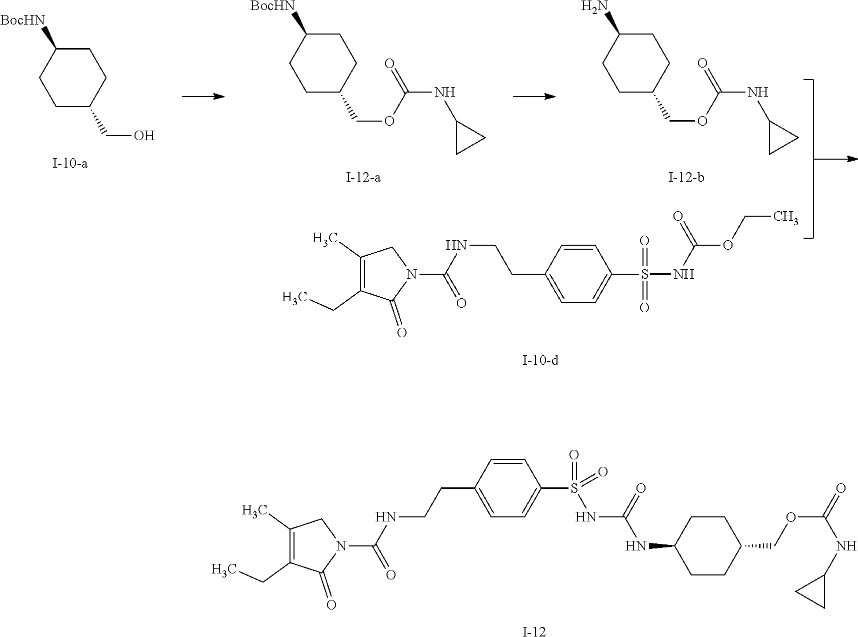

I-12 was prepared with a yield of 52% following the same method as that in Example 10, except that ethyl isocyanate was replaced with isopropyl isocyanate in Step 1. ESI-MS m/z 590.3 (M+H)⁺.

### Example 13

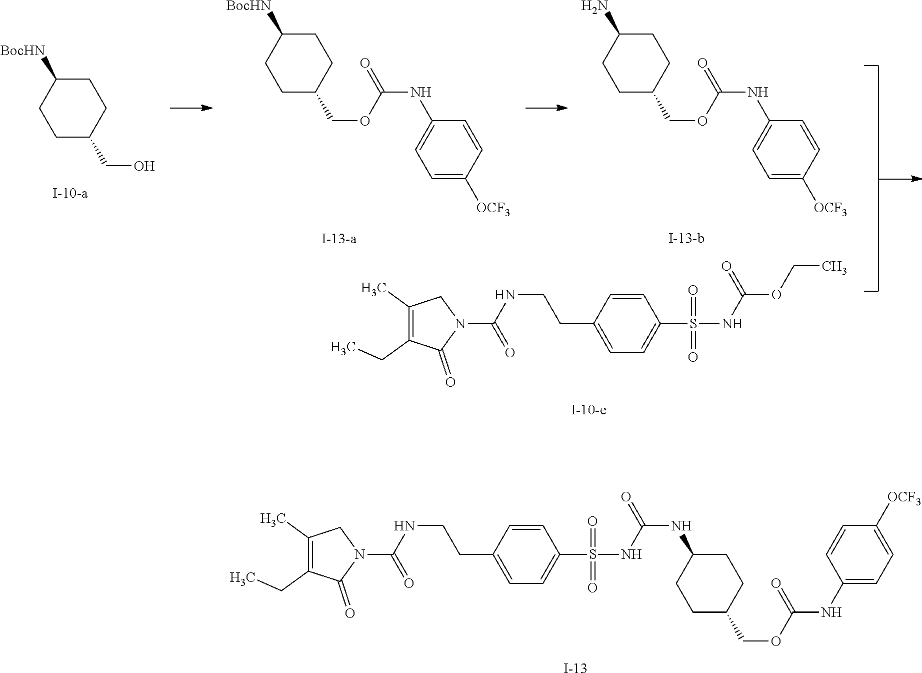

I-13 was prepared with a yield of 62% in the final step following the same method as that in Example 10, except that ethyl isocyanate was replaced with 4-trifluoromethoxyphenyl isocyanate in Step 1. The final step yield was 62%. ¹H NMR (500 MHz, DMSO-d₆) δ 10.37 (s, 1H), 9.82 (s, 1H), 8.37 (t, J=5.8 Hz, 1H), 7.81 (d, J=8.4 Hz, 2H), 7.54 (d, J=8.9 Hz, 2H), 7.46 (d, J=8.4 Hz, 2H), 7.28 (d, J=8.6 Hz, 2H), 6.34 (d, J=7.7 Hz, 1H), 4.17 (s, 2H), 3.89 (d, J=6.5 Hz, 2H), 3.53-3.46 (m, 2H), 3.27-3.16 (m, 1H), 2.90 (t, J=7.2 Hz, 2H), 2.18 (q, J=7.5 Hz, 2H), 2.00 (s, 3H), 1.74 (td, J=14.7, 3.7 Hz, 4H), 1.60-1.49 (m, 1H), 1.12 (qd, J=12.7, 3.7 Hz, 2H), 1.07-0.88 (m, 5H). ESI-MS m/z 710.2 (M+H)⁺.

### Example 14

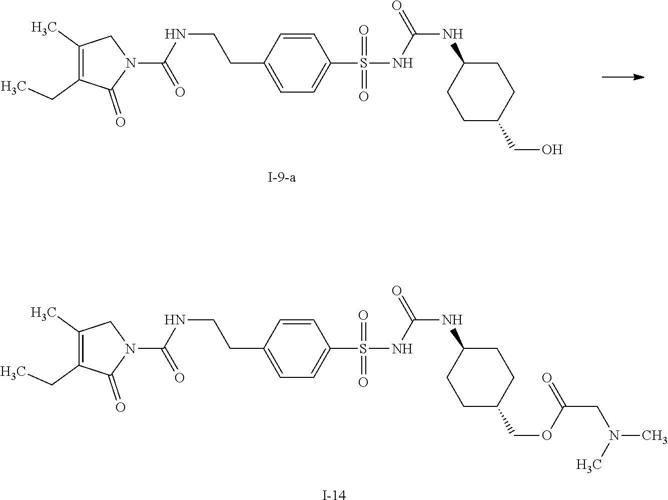

1.1 eq of N,N-dimethylglycine, 1.2 eq of 1-ethyl-3-(3-dimethylaminopropyl)carbodiimide hydrochloride (EDCI·HCl) and a catalytic amount of 4-dimethylaminopyridine (DMAP) were mixed in a small amount of DMF, stirred for 5 minutes, added with I-9-a (50 mg, 1 eq) at once, stirred overnight at room temperature, and diluted with EA. The organic phase was washed with aqueous ammonium chloride and saline, dried, concentrated and purified with silica gel column chromatography to obtain I-14 with a yield of 67%. ¹H NMR (400 MHz, DMSO-d₆) δ 10.54 (s, 1H), 10.21 (s, 1H), 8.38 (t, J=5.8 Hz, 1H), 7.81 (d, J=8.1 Hz, 2H), 7.45 (d, J=8.1 Hz, 2H), 6.63 (d, J=7.7 Hz, 1H), 4.20 (s, 2H), 4.17 (s, 2H), 3.99 (d, J=6.3 Hz, 2H), 3.49 (q, J=6.8 Hz, 2H), 3.22 (d, J=10.4 Hz, 1H), 2.90 (t, J=7.2 Hz, 2H), 2.82 (s, 6H), 2.19 (q, J=7.5 Hz, 2H), 2.01 (s, 3H), 1.78-1.64 (m, 4H), 1.56 (s, 1H), 1.08 (ddd, J=31.6, 13.7, 6.9 Hz, 3H), 0.98 (t, J=7.5 Hz, 4H). ESI-MS m/z 592.5 (M+H)⁺.

### Example 15

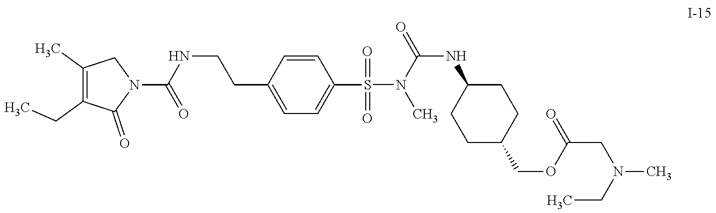 [SMILES: CCC1=C(C)CN(C(=O)NCCc2ccc(S(=O)(=O)N(C)C(=O)N[C@H]3CC[C@H](COC(=O)CN(C)CC)CC3)cc2)C1=O]

I-15 was prepared with a yield of 57% following the same method as that in Example 1, except that I-1-a was replaced with I-14. ESI-MS m/z 606.3 (M+H)⁺.

### Example 16

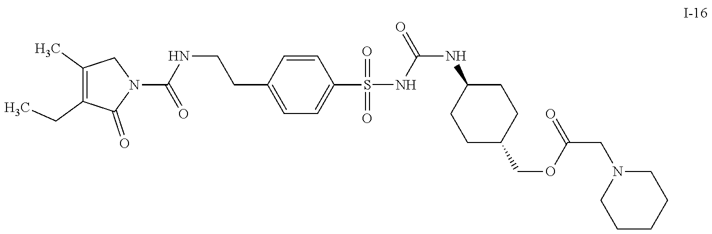 [SMILES: CCC1=C(C)CN(C(=O)NCCc2ccc(S(=O)(=O)NC(=O)N[C@H]3CC[C@H](COC(=O)CN4CCCCC4)CC3)cc2)C1=O]

I-16 was prepared with a yield of 49% following the same method as that in Example 14, except that N,N-dimethylglycine was replaced with 2-(piperidin-1-yl)acetic acid. ESI-MS m/z 632.5 (M+H)⁺.

### Example 17

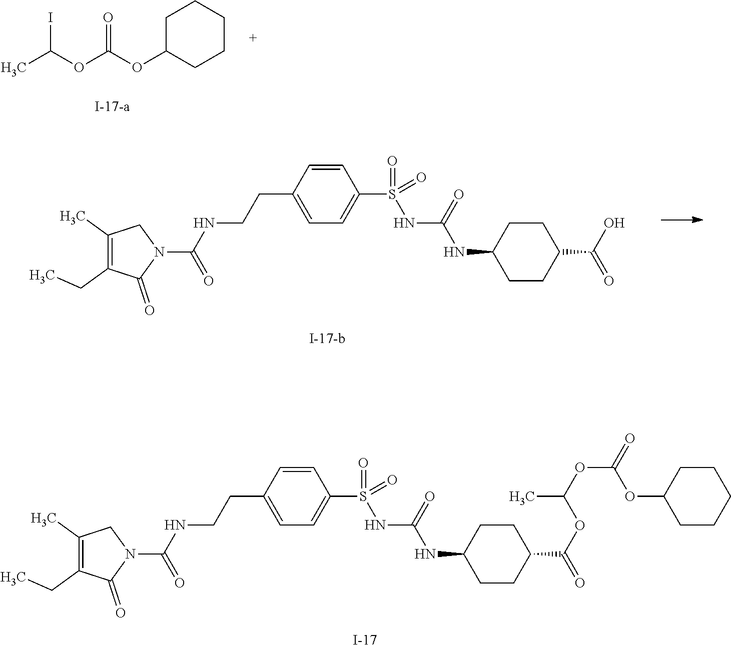

I-17-b and 1.1 eq of I-17-a were dissolved in DMF, added with 2 eq of potassium carbonate, and reacted for 1 hour at room temperature. TLC showed that the reaction was complete. The system was added with water, and extracted several times with EA. The EA phase was washed with water and saturated saline, dried with anhydrous sodium sulfate, filtered and rotary evaporated to dryness, and the residue was purified with column chromatography to obtain I-17 with a yield of 32%. ESI-MS m/z 691.3 (M+H)⁺.

### Example 18

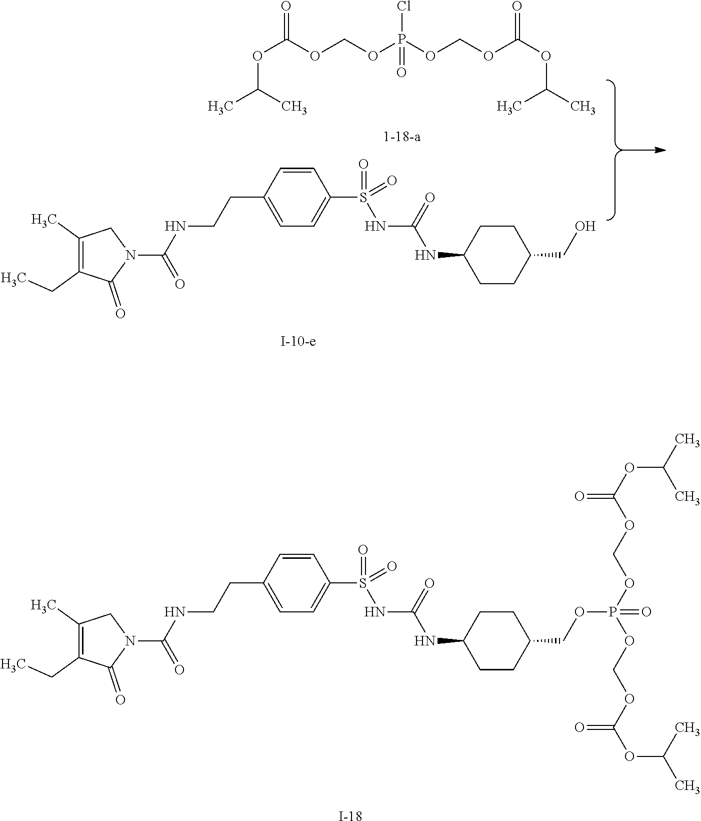

I-10-e (100 mg, 0.2 mmol, 1 eq) was dissolved in DCM (2 mL). After dropwise addition of 2,6-dimethylpyridine (46.6 μL, 0.4 mmol, 2.0 eq), the reaction solution was cooled to −30° C., added with I-18-a (1.5 eq) dissolved in DCM, and then warmed to room temperature. After TLC showed that the reaction was complete, the reaction solution was poured into water (10 mL), extracted with dichloromethane (15 mL×3), and layered. The organic phase was washed with 0.5 M diluted hydrochloric acid (10 mL), saturated sodium bicarbonate (10 mL) and saturated saline (10 mL) successively, dried with anhydrous sodium sulfate, filtered, and rotary evaporated to dryness. The residue was purified with column chromatography to obtain I-18 with a yield of 20% as a white solid. ESI-MS m/z 819.3 (M+H)⁺.

### Example 19

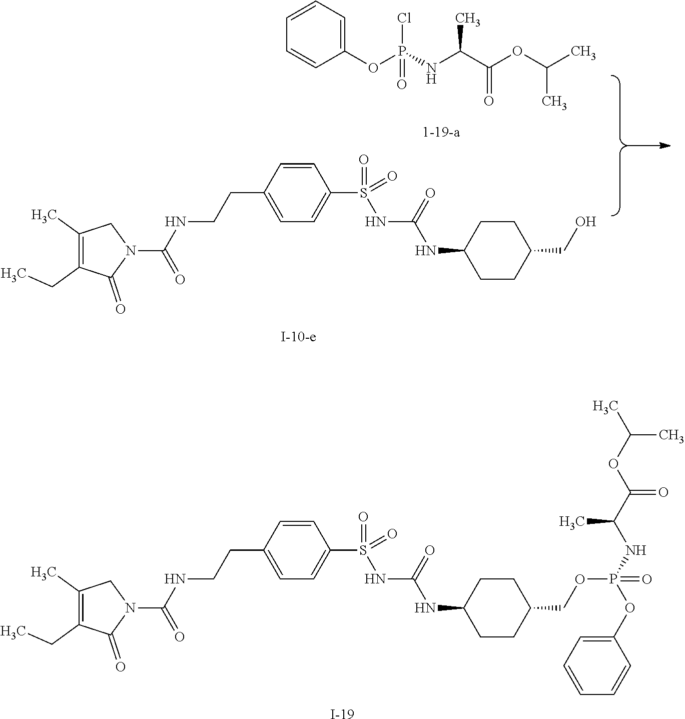

I-19 was prepared with a yield of 19% following the same method as that in Example 18, except that I-18-a was replaced with I-19-a. ESI-MS m/z 776.3 (M+H)⁺.

### Example 20

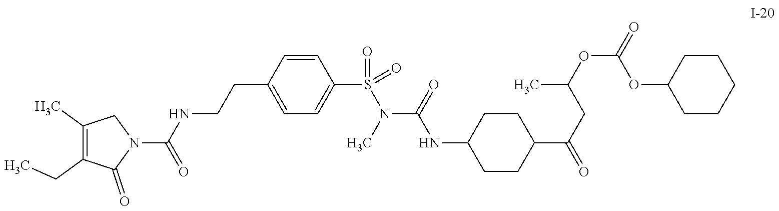 [SMILES: CCC1=C(C)CN(C(=O)NCCc2ccc(S(=O)(=O)N(C)C(=O)NC3CCC(C(=O)CC(C)OC(=O)OC4CCCCC4)CC3)cc2)C1=O]

I-20 was prepared with a yield of 28% following the same method as that in Example 1, except that I-1-a was replaced with 1-17. ESI-MS m/z 705.4 (M+H)⁺.

### Example 21

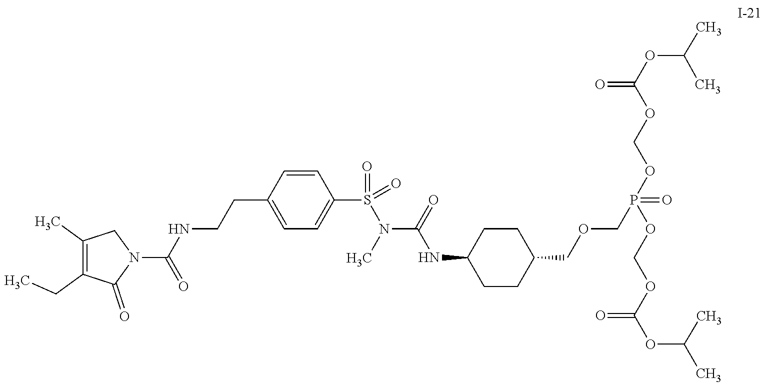 [SMILES: CCC1=C(C)CN(C(=O)NCCc2ccc(S(=O)(=O)N(C)C(=O)N[C@H]3CC[C@H](COCP(=O)(OCOC(=O)OC(C)C)OCOC(=O)OC(C)C)CC3)cc2)C1=O]

I-21 was prepared with a yield of 22% following the same method as that in Example 1, except that I-1-a was replaced with I-18. ESI-MS m/z 847.5 (M+H)⁺.

### Example 22

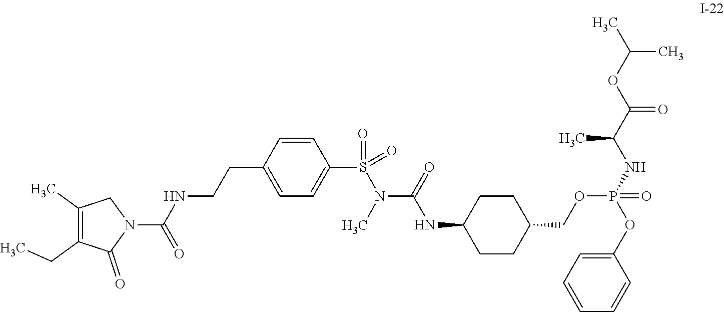 [SMILES: CCC1=C(C)CN(C(=O)NCCc2ccc(S(=O)(=O)N(C)C(=O)N[C@H]3CC[C@H](CO[P@](=O)(N[C@@H](C)C(=O)OC(C)C)Oc4ccccc4)CC3)cc2)C1=O]

I-22 was prepared with a yield of 19% following the same method as that in Example 1, except that I-1-a was replaced with I-19. ESI-MS m/z 790.5 (M+H)⁺.

### Example 23

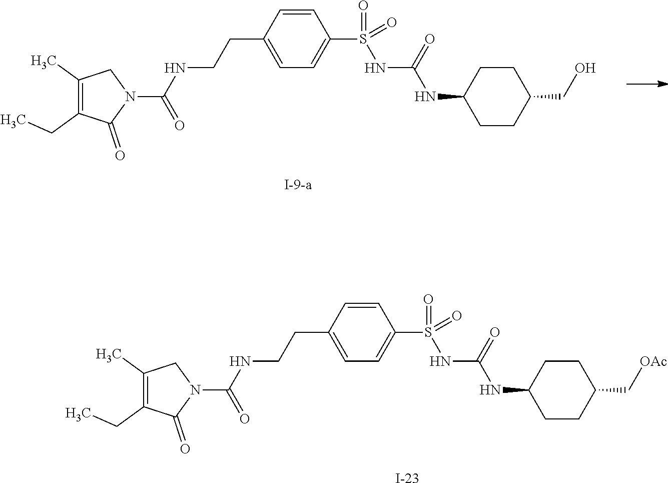

I-9-a (0.14 g, 0.27 mmol) was suspended in dichloromethane, added with N,N-diisopropylethylamine (96 μL, 0.55 mmol) and DMAP (4 mg, 0.03 mmol) at room temperature, added dropwisely with acetic anhydride (39 μL, 0.41 mmol), and stirred overnight. TLC showed that the raw material was completely converted, the reaction mixture was diluted with dichloromethane, and the organic phase was washed with dilute hydrochloric acid and saline, dried with anhydrous sodium sulfate, filtered and concentrated to obtain the crude, which was recrystallized in an appropriate amount of acetone, filtered and dried to obtain I-23 as a white solid with a yield of 57%. ¹H NMR (500 MHz, DMSO-d₆) δ 10.35 (s, 1H), 8.37 (t, J=5.8 Hz, 1H), 7.81 (d, J=8.0 Hz, 2H), 7.46 (d, J=8.0 Hz, 2H), 6.33 (d, J=7.8 Hz, 1H), 4.17 (s, 2H), 3.79 (d, J=6.6 Hz, 2H), 3.50 (q, J=6.7 Hz, 2H), 3.25-3.16 (m, 1H), 2.90 (t, J=7.2 Hz, 2H), 2.18 (q, J=7.5 Hz, 2H), 2.01 (s, 3H), 1.99 (s, 3H), 1.77-1.70 (m, 2H), 1.66 (d, J=13.0 Hz, 2H), 1.50 (s, OH), 1.16-1.05 (m, 2H), 0.97 (t, J=7.4 Hz, 4H). ESI-MS m/z 571.3 (M+Na)⁺.

### Example 24

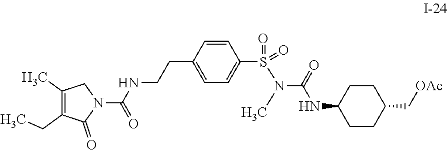 [SMILES: CCC1=C(C)CN(C(=O)NCCc2ccc(S(=O)(=O)N(C)C(=O)N[C@H]3CC[C@H](COC(C)=O)CC3)cc2)C1=O]

I-24 was prepared with a yield of 48% following the same method as that in Example 1, except that I-1-a was replaced with I-23. ESI-MS m/z 585.3 (M+Na)⁺.

### Example 25

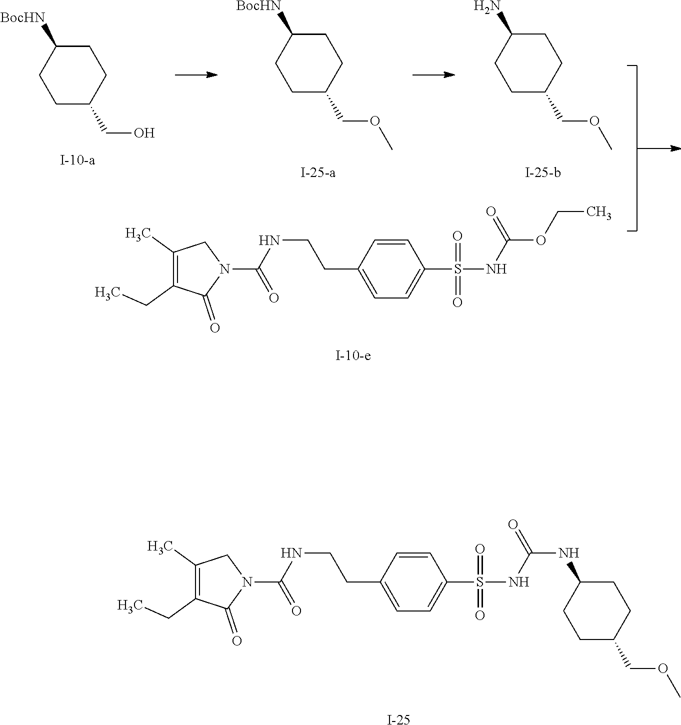

I-25 was prepared with a yield of 78% following the same method as that in Example 10, except that ethyl isocyanate was replaced with iodomethane to obtain I-25-a in Step 1. ESI-MS m/z 521.2 (M+Na)⁺.

### Example 26

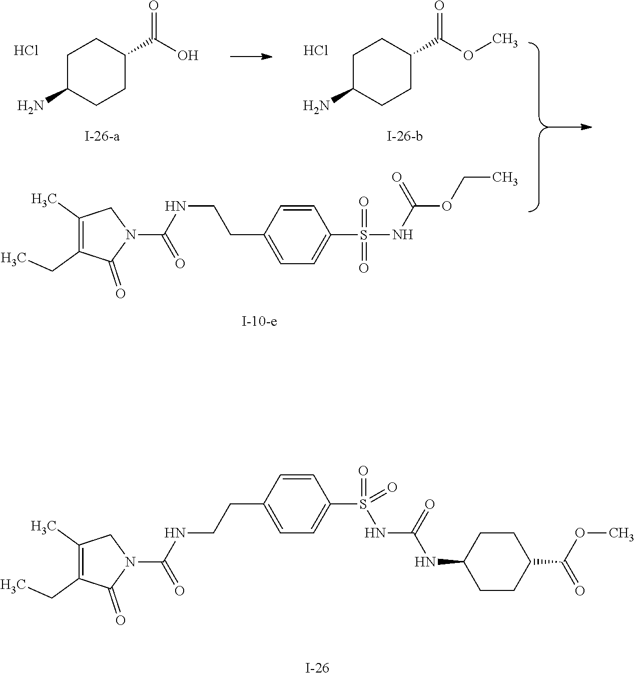

Step 1: The raw material I-26-a (2.5 g, 13.92 mmol) was suspended in 20 mL of methanol, added dropwisely with concentrated sulfuric acid (275 mg, 2.8 mmol), and reacted overnight at 60° C. The solvent was rotary evaporated off to form a large number of white solid, which was slurried in 15 mL of acetone, filtered, and dried to obtain I-26-b as a white solid with a yield of 86%.

Step 2: I-26 was prepared with a yield of 72% by reacting I-26-b with I-10-e following the same method as that in Step 4 of Example 10. ¹H NMR (500 MHz, DMSO-d₆) δ 10.37 (s, 1H), 8.37 (t, J=5.8 Hz, 1H), 7.86-7.74 (m, 1H), 7.46 (d, J=8.5 Hz, 1H), 6.35 (d, J=7.7 Hz, 1H), 4.17 (s, 1H), 3.56 (s, 2H), 3.52-3.47 (m, 1H), 3.22 (dt, J=7.7, 3.9 Hz, OH), 2.90 (t, J=7.2 Hz, 1H), 2.27-2.13 (m, 2H), 2.08 (s, 1H), 2.01 (s, 1H), 1.89-1.81 (m, 1H), 1.73 (dd, J=12.8, 3.6 Hz, 1H), 1.31 (qd, J=13.2, 3.4 Hz, 1H), 1.20-1.08 (m, 1H), 0.97 (t, J=7.5 Hz, 2H). ESI-MS m/z 535.4 (M+H)⁺.

### Example 27

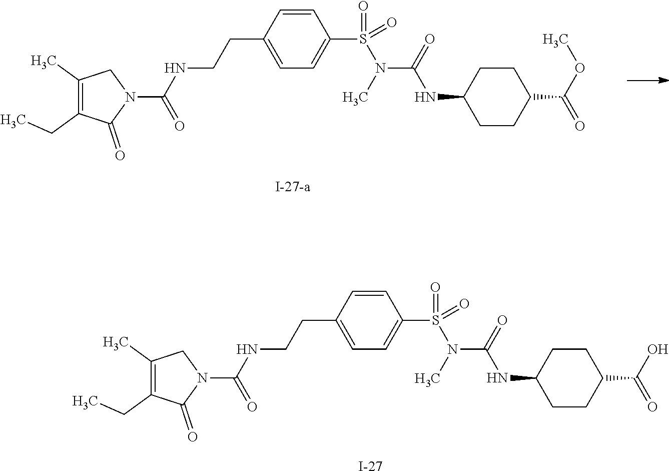

Step 1: I-27-a was prepared with a yield of 48% following the same method as that in Example 1, except that I-1-a was replaced with I-26.

Step 2: I-27-a (55 mg, 0.1 mmol) was suspended in 1 mL of tetrahydrofuran, added with lithium hydroxide monohydrate (0.12 mmol) dissolved in 0.1 mL of water, added with 0.1 mL of methanol, and refluxed for 3 hours. The solvent was rotary evaporated off, and water was added. The reaction mixture was adjusted pH with 1 M hydrochloric acid, and carefully added with 1.2 mL of 1 M hydrochloric acid to precipitate a large amount of solid, which was filtered to obtain I-27 with a yield of 48%. ESI-MS m/z 557.2 (M+Na)⁺.

### Example 28

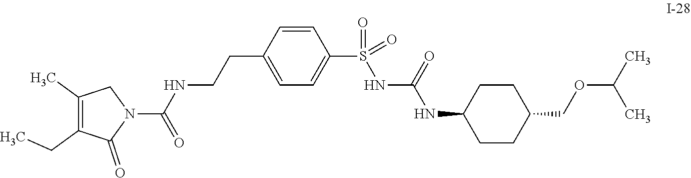 [SMILES: CCC1=C(C)CN(C(=O)NCCc2ccc(S(=O)(=O)NC(=O)N[C@H]3CC[C@H](COC(C)C)CC3)cc2)C1=O]

I-28 was prepared with a yield of 65% following the same method as that of Example 26, except that methanol was replaced with isopropanol in Step 1. ESI-MS m/z 563.3 (M+H)⁺.

### Example 29

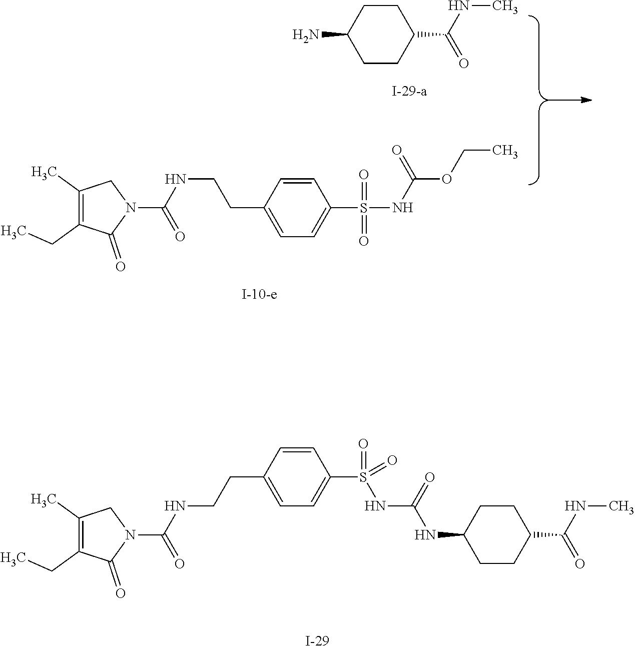

I-29 was prepared with a yield of 58% following the same method as that in Step 4 of Example 10, except that I-10-c was replaced with I-29-a. ESI-MS m/z 534.5 (M+H)⁺.

### Example 30

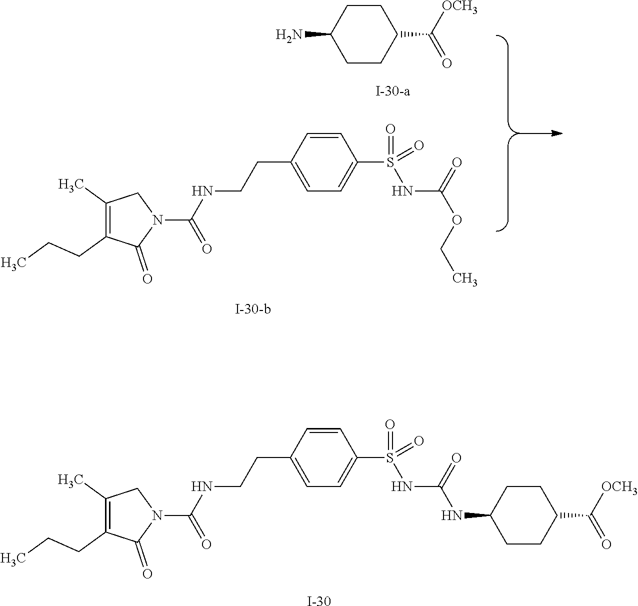

I-30 was prepared with a yield of 38% following the same method as that in Step 4 of Example 10, except that I-10-c was replaced with I-30-a and I-10-e was replaced with I-30-b. ESI-MS m/z 549.2 (M+H)⁺.

### Example 31

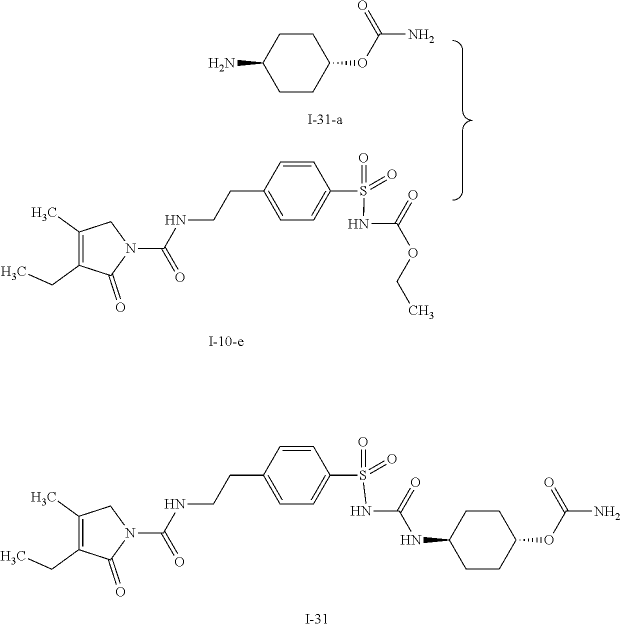

I-31 was prepared with a yield of 42% following the same method as that in Step 4 of Example 10, except that I-10-c was replaced with I-31-a. ESI-MS m/z 536.2 (M+H)⁺.

### Example 32

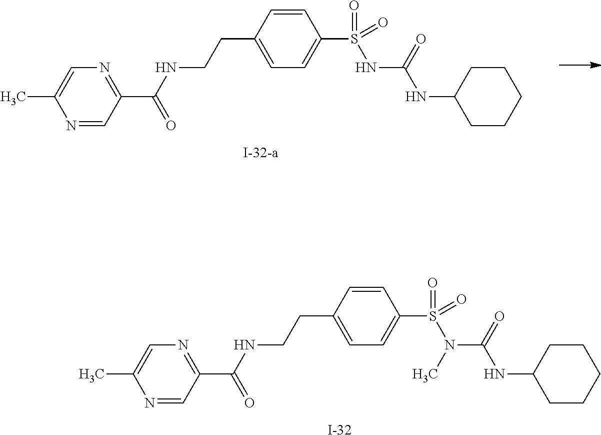

I-32 was prepared with a yield of 25% following the same method as that in Example 1, except that I-1-a was replaced with I-32-a. ESI-MS m/z 460.2 (M+H)⁺.

### Example 33

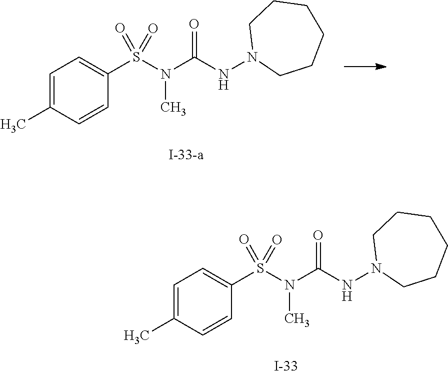

I-33 was prepared with a yield of 35% following the same method as that in Example 1, except that I-1-a was replaced with I-33-a. ESI-MS m/z 326.2 (M+H)⁺.

### Example 34

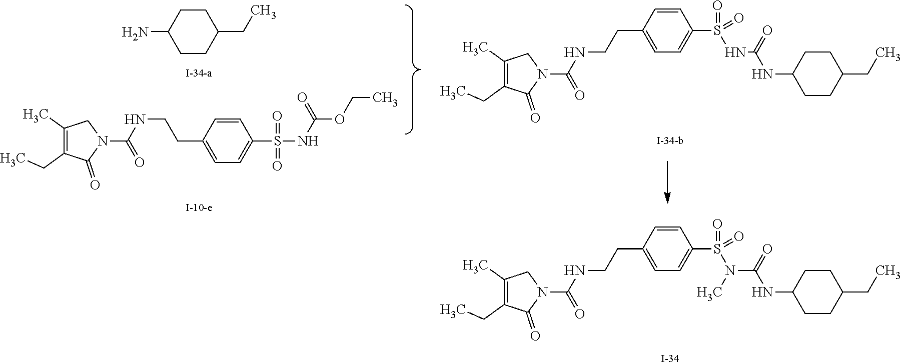

Step1: I-34-b was prepared following the same method as that in Step 4 of Example 10, except that I-10-c was replaced with I-34-a.

Step2: I-34 was prepared with a yield of 35% following the same method as that in Example 1, except that I-1-a was replaced with I-34-a. ESI-MS m/z 519.3 (M+H)⁺.

### Example 35

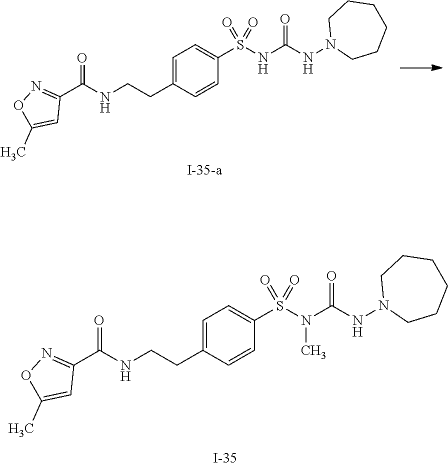

I-35 was prepared with a yield of 33% following the same method as that in Example 1, except that I-1-a was replaced with I-35-a. ESI-MS m/z 464.2 (M+H)⁺.

### Example 36

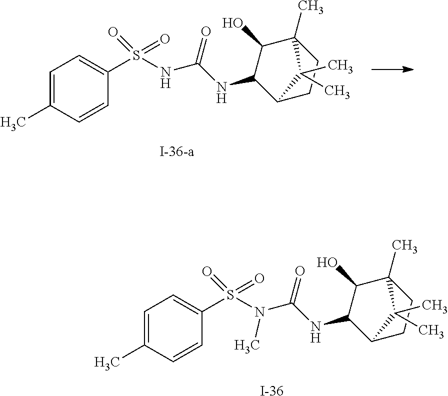

I-36 was prepared with a yield of 38% following the same method as that in Example 1, except that I-o-a was replaced with I-36-a. ES-MS m/z 381.1(M+H)⁺.

### Example 37

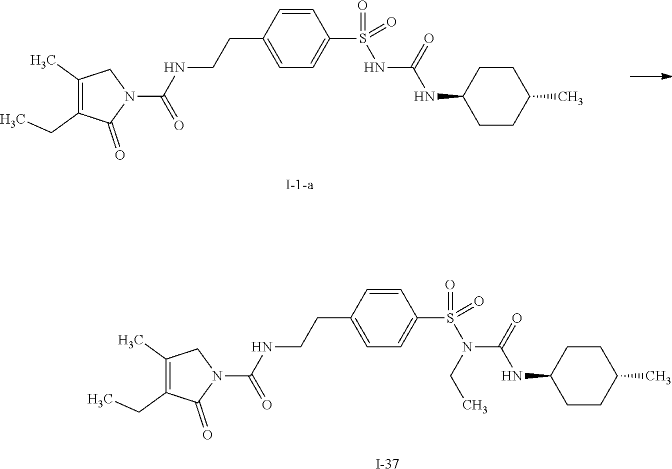

I-37 was prepared with a yield of 39% following the same method as that in Example 1, except that iodomethane was replaced with iodoethane. ¹H NMR (400 MHz, DMSO-d₆) δ 8.35 (t, J=5.8 Hz, 1H), 7.84-7.74 (m, 2H), 7.53-7.45 (m, 2H), 7.27 (d, J=7.7 Hz, 1H), 4.15 (s, 2H), 3.60 (q, J=7.0 Hz, 2H), 3.57-3.46 (m, 2H), 3.36 (dt, J=7.8, 3.9 Hz, OH), 2.91 (t, J=7.1 Hz, 2H), 2.18 (q, J=7.5 Hz, 2H), 2.01 (s, 3H), 1.73 (dd, J=13.0, 3.8 Hz, 2H), 1.67-1.59 (m, 2H), 1.25 (qd, J=12.7, 3.4 Hz, 3H), 1.05 (t, J=7.0 Hz, 3H), 0.97 (t, J=7.5 Hz, 3H), 0.92 (dd, J=12.0, 3.1 Hz, 1H), 0.84 (d, J=6.5 Hz, 3H). ESI-MS m/z 518.8 (M+H)⁺.

### Example 38

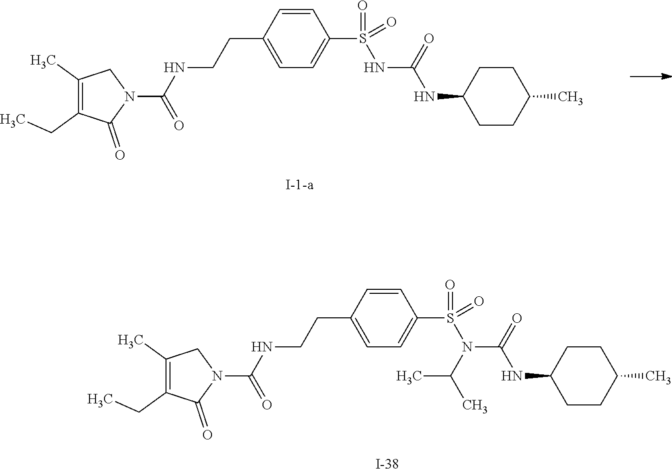

I-38 was prepared with a yield of 21.3% following the same method as that in Example 1, except that iodomethane was replaced with 2-iodopropane. ESI-MS m/z 533.5 (M+H)⁺.

### Example 39

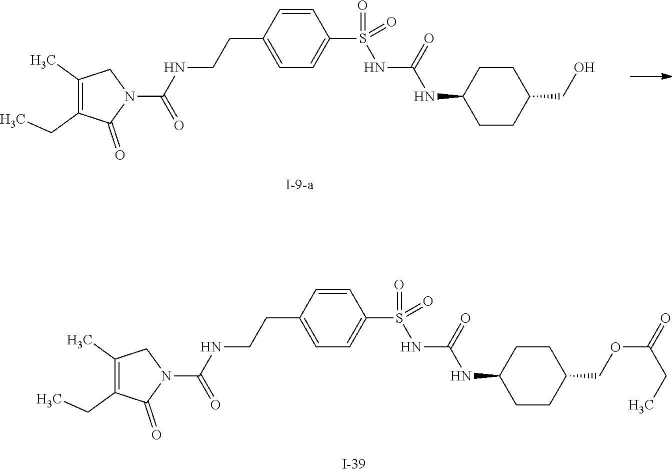

I-39 was prepared with a yield of 42% following the same method as that in Example 23, except that acetic anhydride was replaced with propionic anhydride. ESI-MS m/z 563.2 (M+H)⁺.

### Example 40

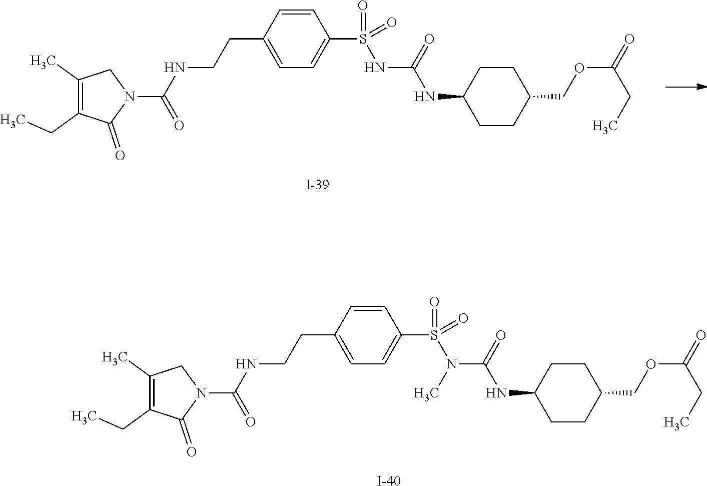

I-40 was prepared with a yield of 38% following the same method as that in Example 1, except that I-1-a was replaced with I-39. ESI-MS m/z 599.3 (M+Na)⁺.

### Example 41

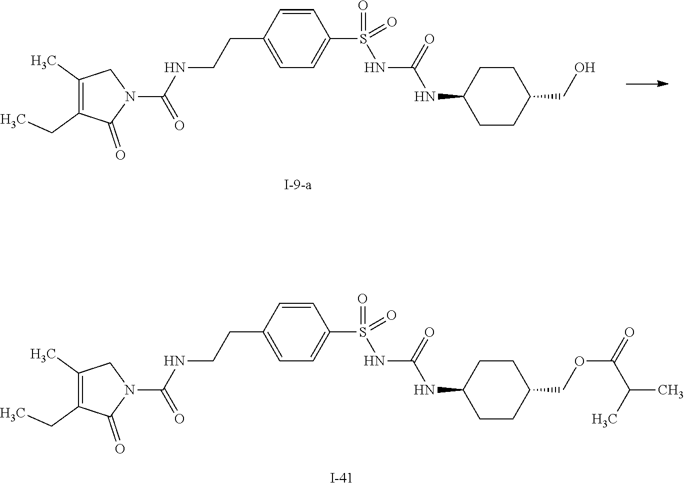

I-41 was prepared with a yield of 35% following the same method as that in Example 23, except that acetic anhydride was replaced with isobutyric anhydride. ESI-MS m/z 577.2 (M+H)⁺.

### Example 42

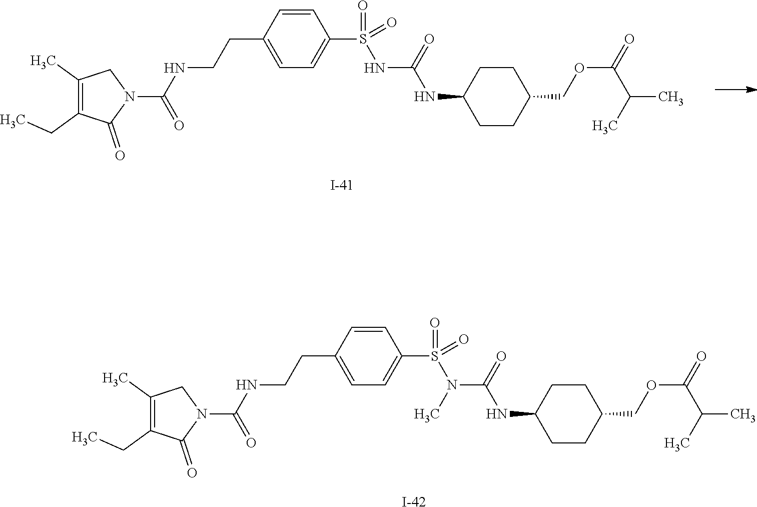

I-42 was prepared with a yield of 41% following the same method as that in Example 1, except that I-1-a was replaced with I-41. ESI-MS m/z 591.3 (M+H)⁺.

### Example 43

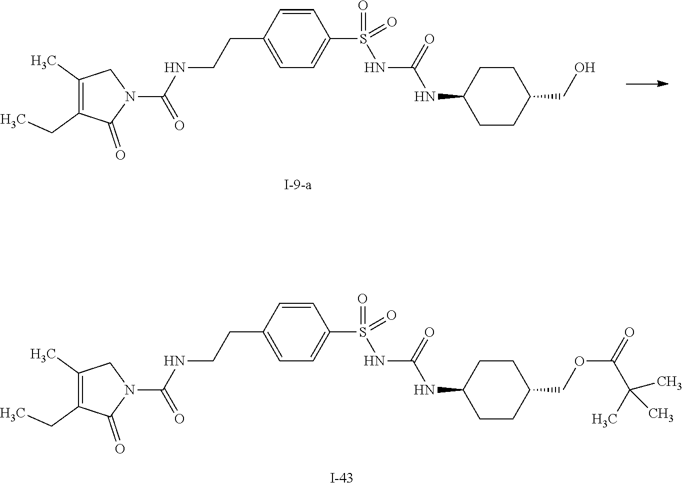

I-43 was prepared with a yield of 37% following the same method as that in Example 23, except that acetic anhydride was replaced with pivalic anhydride. ESI-MS m/z 591.3 (M+H)⁺.

### Example 44

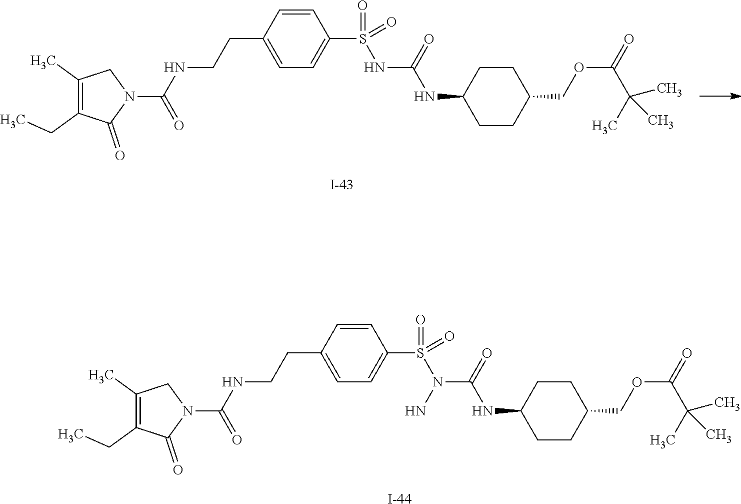

I-44 was prepared with a yield of 45% following the same method as that in Example 1, except that I-1-a was replaced with I-43. ESI-MS m/z 605.3 (M+H)⁺.

### Experimental Example 1 sEH Inhibition Assay

sEH Assay Buffer was purchased from Cayman Chemical; sEH (human recombinant) was purchased from Cayman Chemical; sEH substrate was purchased from Cayman Chemical. The working solution of 20% DMSO for the compounds was prepared by mixing dimethyl sulfoxide with water in a volume ratio of 1:4, and shaking to mix well.

### Experimental Method:

sEH Assay Buffer (10×), SEH (human recombinant) and sEH substrate were thawed on ice.

The sEH Assay Buffer (lux) was prepared into a 1×sEH Assay Buffer with deionized water. 12 μL of sEH was added with 588 L of 1× Assay Buffer to be diluted 50 times to obtain a sEH working solution. 180 L of 1× Assay Buffer was added into the corresponding wells; the sEH working solution was added to the corresponding wells at 5 μL/well; the working solution of 20% DMSO for the compounds was added into the corresponding wells at 5 μL/well, and blank wells and negative control wells added with an equal amount of DMSO were set. After centrifuged at 1000 rpm for 1 minute, the wells were incubated at 25° C. for 30 minutes. After incubation, the substrate was added into the 96-wells plate at 5 μL/well and the plate was centrifuged at 1000 rpm for 1 minute. After incubation at 25° C. for 15 minutes, fluorescence values were read at emission wavelengths of 320 nm and 460 nm.

### Calculation of Enzymic Inhibition Rate:

$$Inhibition rate ( \% ) = 10 0- ( Fluorescence intensity ( sample ) - Fluorescence intensity ( blank ) ) / ( Fluorescence intensity ( negative control ) - Fluorescence intensity ( blank ) ) \times 100 \%$$

The experimental results were shown in Table 1, wherein A indicates an IC₅₀ less than 200 nM, B indicates an IC₅₀ between 200 nM and 1 μM, C indicates an IC₅₀ between 1 μM and 10 μM, and D indicates an IC₅₀ between 10 μM to 100 μM.

TABLE 1
Inhibitory activities of compounds on sEH

| Compound number | Inhibitory activity |
|---|---|
| I-1 | C |
| I-2 | A |
| I-3 | A |
| I-4 | C |
| I-5 | B |
| I-6 | C |
| I-7 | C |
| I-8 | C |
| I-9 | B |
| I-10 | B |
| I-11 | A |
| I-12 | B |
| I-13 | C |
| I-14 | D |
| I-15 | B |
| I-16 | B |
| I-17 | D |
| I-18 | D |
| I-19 | D |
| I-20 | D |
| I-21 | D |
| I-22 | D |
| I-23 | C |
| I-24 | C |
| I-25 | B |
| I-26 | C |
| I-27 | D |
| I-28 | B |
| I-29 | C |
| I-30 | D |
| I-31 | C |
| I-32 | C |
| I-33 | C |
| I-34 | C |
| I-35 | C |
| I-36 | C |
| I-37 | C |
| I-38 | C |
| I-39 | C |
| I-40 | C |
| I-41 | C |
| I-42 | C |
| I-43 | C |
| I-44 | C |

### Experimental Example 2 Activity Assay of Compounds

The compounds I-1, I-4 and I-23 of the present invention and glimepiride were used for the assay, and 12-(3-adamantan-1-yl-ureido)dodecanoic acid (AUDA) was used as the positive control.

Biomaterial sources: Human cardiomyocyte cell line AC16 was purchased from ATCC, USA. Phenylephrine (PE) was purchased from Sigma Company. AUDA was purchased from Cayman Company.

### (1) Inhibition Assay on sEH in Cardiomyocytes

### Cell Incubation Method:

The compounds were dissolved in DMSO. The working concentrations were 0.1, 1 and 10 μM for the compounds of the present invention, 0.1, 1 and 10 μM for glimepiride, and 1 μM for AUDA. The incubation time was 24 hours.

### Activity Detection of sEH:

The ELISA detection kit (BIOTARGET 14,15-EET/DHET ELISA KIT) from Detroit R&D company was used, and the experiment was conducted according to the instructions of the kit.

### Experiment:

After 24 hours of incubation, cardiomyocytes were collected to perform the above detection.

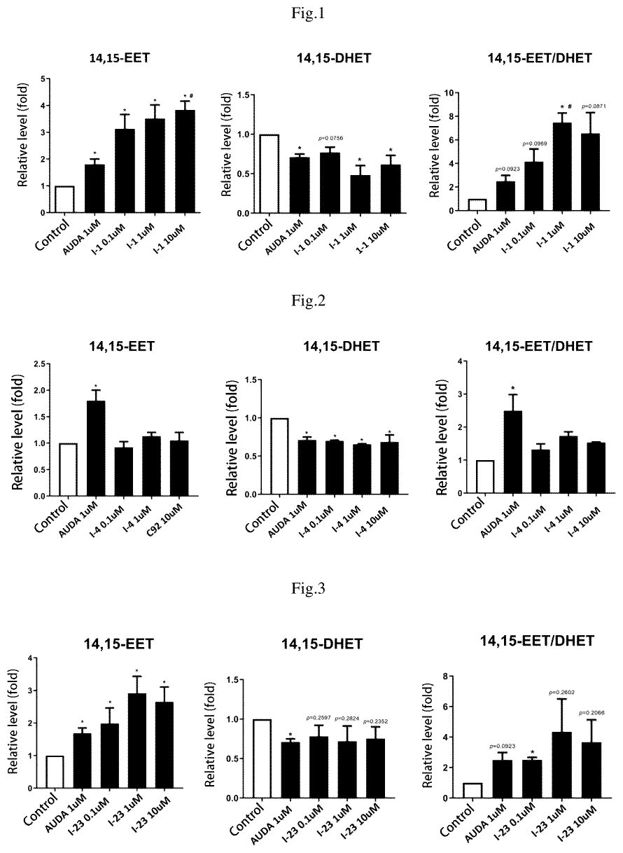

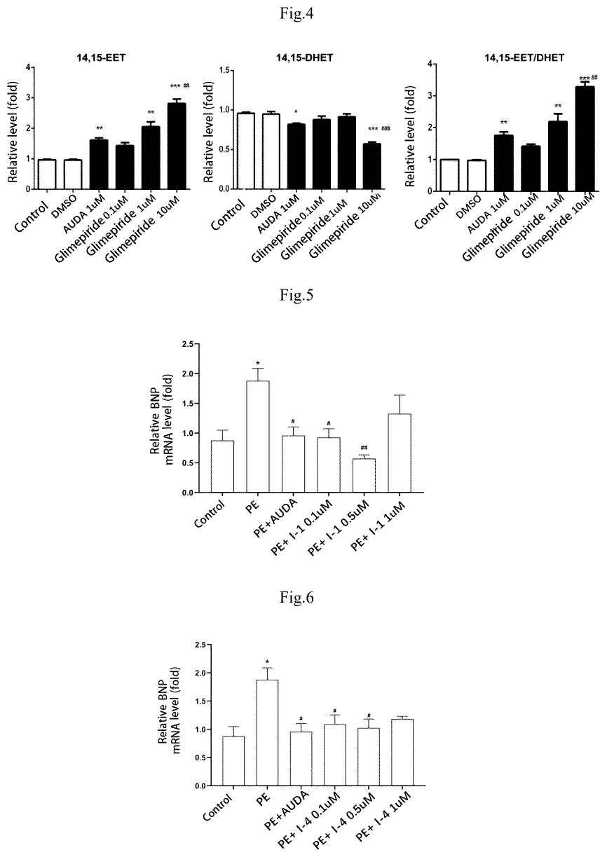

The experimental results are shown in FIGS. 1, 2, 3 and 4, wherein FIG. 1 shows the inhibitory effect of I-1 on sEH, FIG. 2 shows the inhibitory effect of I-4 on sEH, FIG. 3 shows the inhibitory effect of I-23 on sEH, and FIG. 4 shows the inhibitory effect of glimepiride on sEH.

It can be seen from FIGS. 1, 2 and 3 that, compared with the control, I-1, I-4, and I-23 of the present invention significantly increase the content of EETs while reducing the content of DHET, indicating that I-1, I-4, and I-23 inhibit the activity of sEH in cardiomyocytes.

It can be seen from FIG. 4 that, glimepiride has a significant inhibitory effect on enzymic activity of sEH, and the inhibitory effect increases as the concentration increases.

### (2) Inhibitory Effect on PE Induced Myocardial Injury

### Cell Incubation Method:

PE was dissolved in disinfected deionized water with a working concentration of 100 μM. The compounds were dissolved in DMSO, and the working concentrations were 0.1, 0.5, 1 μM for the compounds of the present invention, 10 μM for glimepiride, and 1 μM for AUDA. The incubation time was 24 hours.

Ventricular Remodeling Biomarker mRNA Detection:

Total RNA was extracted using Invitrogen's Trizol reagent, then cDNA was obtained using TAKARA's reverse transcription kit, and finally the mRNA content of related genes was amplified with specific primers.

### Experimental Results:

After 24 hours of incubation, cardiomyocytes were collected to perform the above detection.

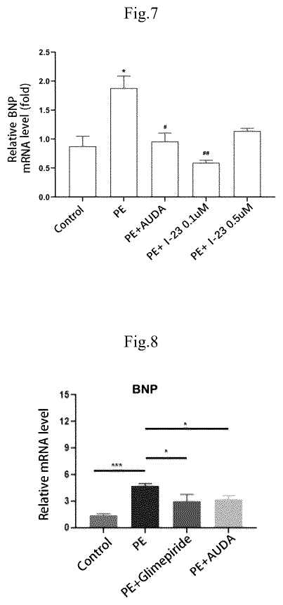

The results are shown in FIGS. 5, 6, 7 and 8, wherein FIG. 5 shows the inhibitory effect of I-1 on PE induced cardiomyocyte remodeling, FIG. 6 shows the inhibitory effect of I-4 on PE induced cardiomyocyte remodeling, FIG. 7 shows the inhibitory effect of I-23 on PE induced cardiomyocyte remodeling, and FIG. 8 shows the inhibitory effect of glimepiride on PE induced cardiomyocyte remodeling.

It can be seen from FIGS. 5, 6, 7 and 8 that, compared with the control, BNP expression induced by PE are increased, indicating that remodeling of cardiomyocytes occurs, and the compounds I-1, I-4, and I-23 of the present invention and glimepiride can inhibit this effect.

### (3) Anti-Heart Failure Effect In Vivo

### Establishment of Mouse Model:

12-week-old male C57BL/6 mice, which were purchased from Gempharmatech Co., Ltd, were raised in the SPF animal house of Tongji Medical College, Huazhong University of Science and Technology. After one week of acclimatization in the animal house, the mice were subjected to thoracic aortic constriction as the TAC group. Another group of mice were subjected to thoracotomy without ligation of thoracic aorta as Sham group. Meanwhile, AUDA was used as the positive control.

### Ultrasonic Detection of Mouse Heart:

Cardiac ultrasound detection was performed on a Visual Sonics Vevo 2100 small animal ultrasound imager. The major measurement indicators include heart rate (HR), left ventricular ejection fraction (LVEF), left ventricular fractional shortening (LVFS), left ventricular internal dimension-diastole (LVIDd) and left ventricular internal dimension-systole (LVIDs), and left ventricular posterior wall thickness (LVPWd and LVPWs).

### Hemodynamic Detection of Mouse Heart:

The detection was performed on a Millar pressure-volume system from Millar Instr Ment PowerLab. After the animals were anesthetized to an appropriate extent, a midline incision was made in the neck, the right common carotid artery was separated, ligated at the distal end and clipped at the proximal end. A V-shaped incision was made on the artery with microscissors, and a microcatheter was inserted to the left ventricle. Signals were recorded through a conduction system to obtain hemodynamic data such as heart rate (HR), left ventricular end diastolic pressure (PED), left ventricular end systolic pressure (PES), maximum rate of decrease in left ventricular pressure (−dp/dtmin), and maximum rate of increase in left ventricular pressure (+dP/dtmax).

### Histological Detection:

The myocardial tissue was placed in an embedding frame, soaked and fixed in neutral formaldehyde solution, dehydrated, and embedded in paraffin. The paraffin block was then sliced into sections with a thickness of 4 μm on a microtome. The general appearance of the myocardium was observed through HE staining, and the myocardial fibrosis was observed through Sirius red staining.

Ventricular Remodeling Biomarker mRNA Detection:

Total RNA was extracted using Invitrogen's Trizol reagent, then cDNA was obtained using TAKARA's reverse transcription kit, and finally the mRNA content of related genes was amplified with specific primers.

### Experiment:

Cardiac ultrasound examination was performed two weeks after TAC surgery. 24 TAC mice with no significant difference in cardiac function were selected and randomly divided into a model group (TAC) and a treatment group (TAC+compound). The model group received no other treatment, while the treatment group received daily gavage (0.2 mg/kg and 0.4 mg/kg for I-1 and I-23 of the present application, and 1.2 mg/kg for glimepiride). Eight weeks later, i.e., at the chronic heart failure phase, the animals were sacrificed, and tissue specimens were collected to perform the above detections.

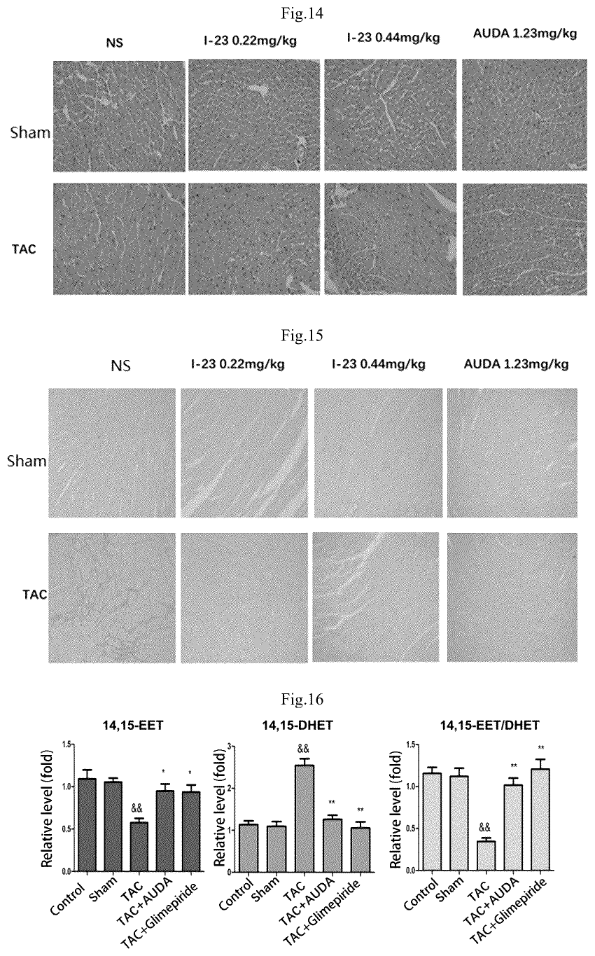

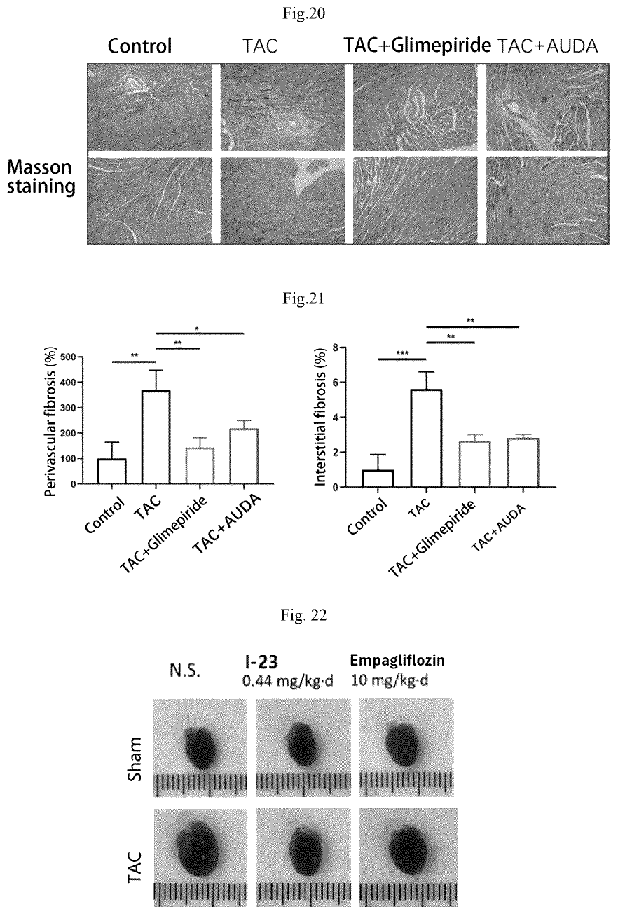

The results are shown in FIGS. 9-21, wherein FIGS. 9-12 shows the results of I-1, FIGS. 13-15 shows the results of I-23, and FIGS. 16-21 shows the results of glimepiride (wherein Sham represents Sham group).

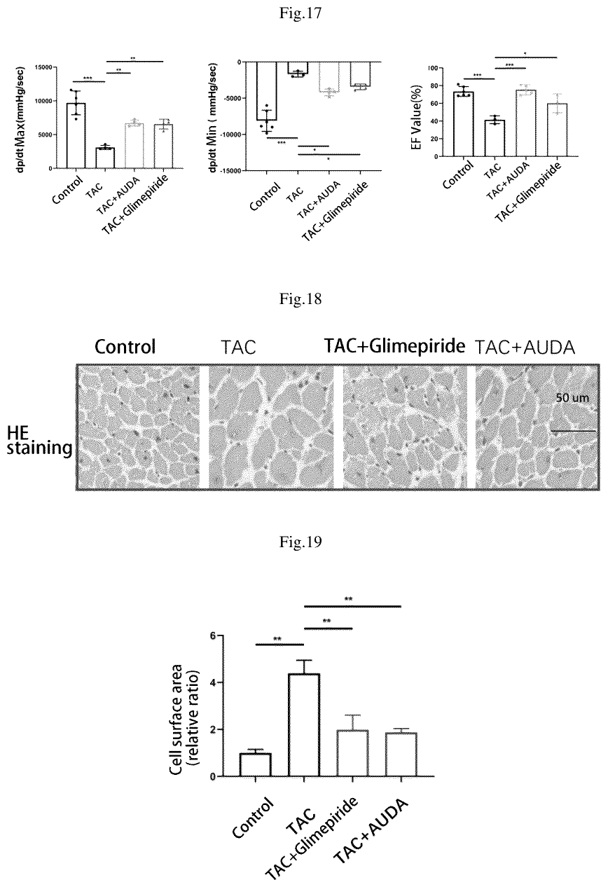

From FIGS. 9 and 13, it can be seen that, compared with the control group, TAC surgery significantly reduced the ejection fraction (EF) value, FS value and dp/dt of the heart in mice with chronic heart failure, indicating impaired cardiac function, and I-1 and I-23 can inhibit this effect. From FIG. 16, it can be seen that, compared with the control group, TAC surgery increased sEH enzymic activity in the hearts of mice with chronic heart failure, and glimepiride could inhibit this effect. From FIG. 17, it can be seen that TAC surgery significantly reduced the ejection fraction (EF) value and dp/dt of the heart in mice with chronic heart failure, indicating impaired cardiac function, while glimepiride can inhibit this effect.

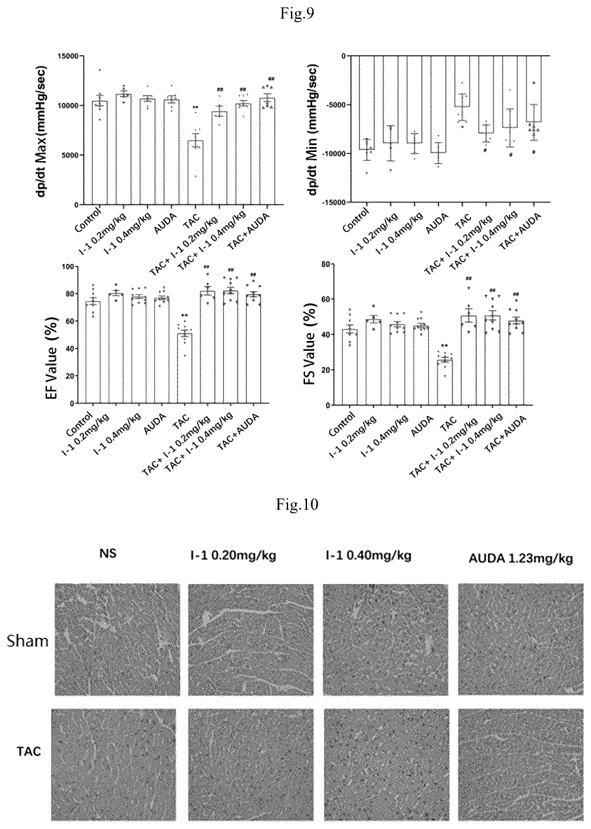

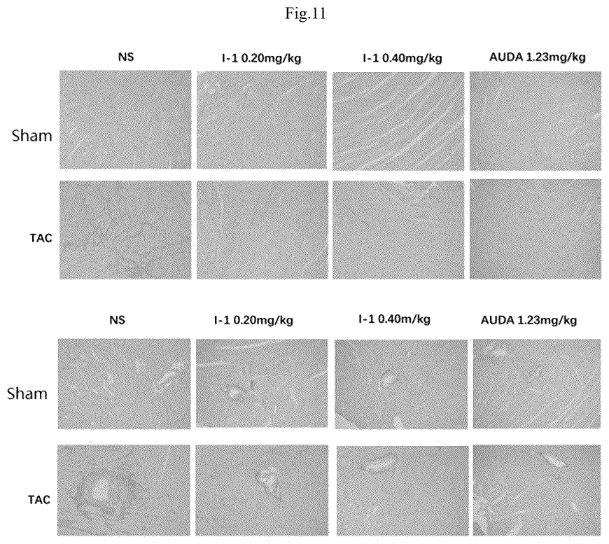

From FIGS. 10, 14, 18 and 19, it can be seen that, HE staining shows that TAC surgery increased the cross-sectional area of cardiomyocytes in mice with chronic heart failure, indicating myocardial hypertrophy, and I-1, I-23, and glimepiride can inhibit this effect.

From FIGS. 11, 15, 20, and 21, it can be seen that, TAC surgery increased the positive area of Sirius red staining in the hearts of mice with chronic heart failure, indicating the occurrence of myocardial fibrosis, and I-1, I-23, and glimepiride can inhibit this effect.

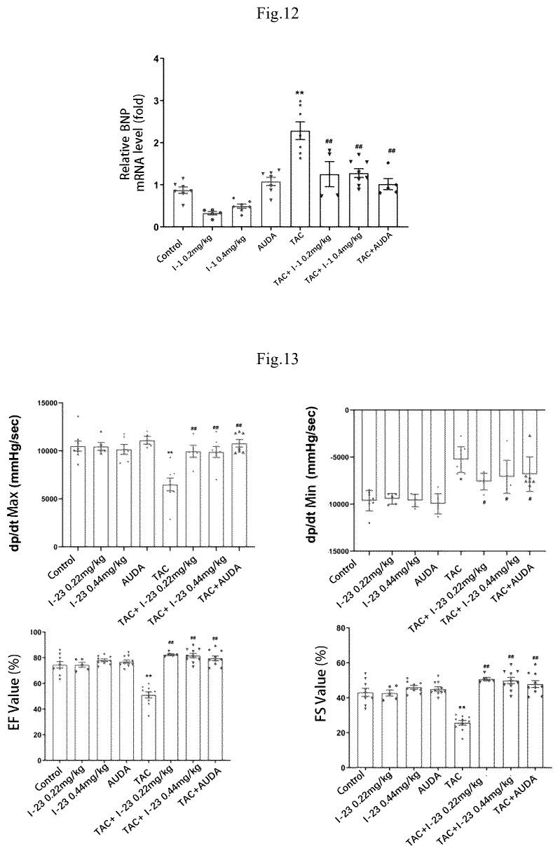

From FIG. 12, it can be seen that, TAC surgery increased the expression of BNP in mouse cardiomyocytes, indicating remodeling of cardiomyocytes, and I-1 can inhibit this effect.

### Experiment:

Cardiac ultrasound examination was performed two weeks after TAC surgery. 24 TAC mice with no significant difference in cardiac function were selected and randomly divided into a model group (TAC) and a treatment group (TAC+compound). The model group received no other treatment, while the treatment group received daily gavage (0.44 mg/kg for I-23 or 10 mg/kg for Empagliflozin). Eight weeks later, i.e., at the chronic heart failure phase, the animals were sacrificed, and tissue specimens were collected to perform the above detections.

This experiment was to compare the ameliorative effects of I-23 and Empagliflozin on chronic heart failure.

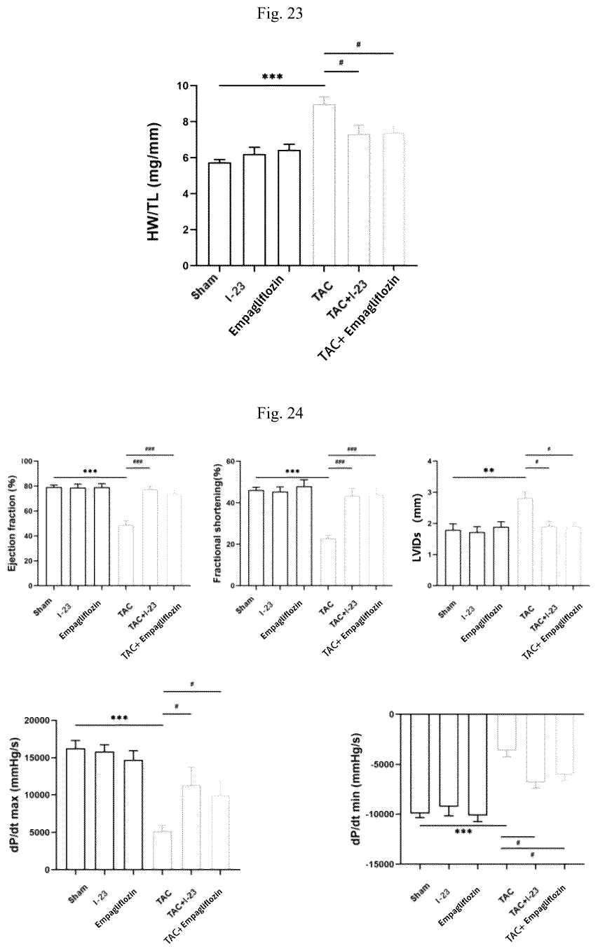

From FIGS. 22 and 23, it can be seen that I-23 and Empagliflozin have a reversal effect on cardiac hypertrophy in mice with chronic heart failure, and I-23 has an effect comparable to that of Empagliflozin.

From FIG. 24, it can be seen that, compared with the control group, TAC surgery significantly reduced the ejection fraction (EF) value, FS value, systolic left ventricular diameter LVIDs, and dp/dt in mice with chronic heart failure, indicating impaired cardiac function, and I-23 and Empagliflozin can inhibit this effect, and I-23 has an effect comparable to that of Empagliflozin.

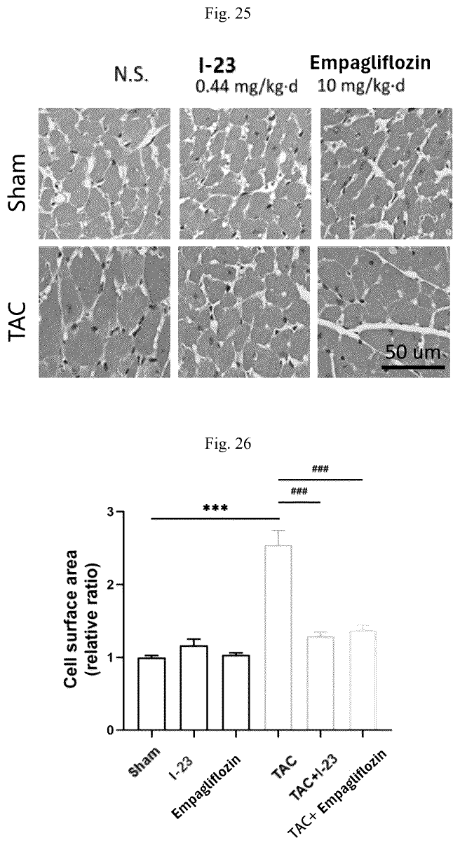

From FIGS. 25 and 26, it can be seen that, HE staining showed that TAC surgery increased the cross-sectional area of cardiomyocytes in mice with chronic heart failure, indicating myocardial hypertrophy, and both of I-23 and Empagliflozin can improve this effect.

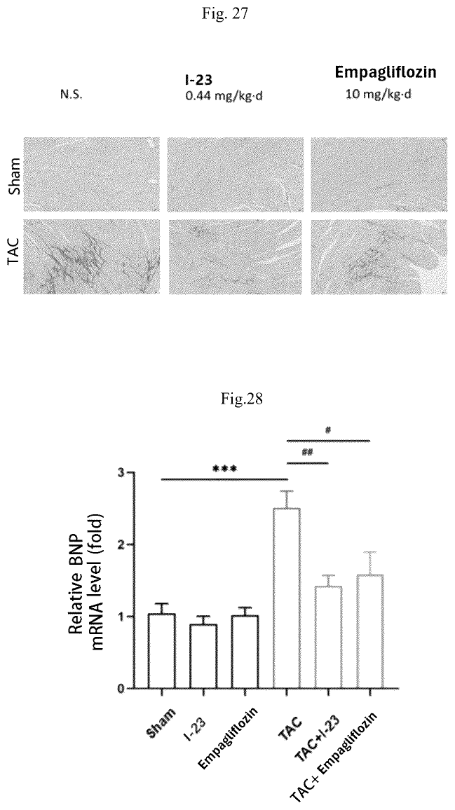

From FIG. 27, it can be seen that, TAC surgery increased the positive area of Sirius red staining in the hearts of mice with chronic heart failure, indicating the occurrence of myocardial fibrosis, and I-23 and Empagliflozin can inhibit this effect.

From FIG. 28, it can be seen that, TAC surgery increased the expression of BNP in mouse cardiomyocytes, indicating remodeling of cardiomyocytes, and I-23 and Empagliflozin can inhibit this effect.

The administration concentration of AUDA that was not specifically specified in the listed experimental examples was 1 μM.

1. A sulfonylurea compound of formula I, or a stereoisomer or pharmaceutically acceptable salt thereof: 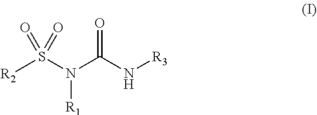
wherein,
R₁ is H,
R₂ is a C6-C14 aryl substituted with —(CH₂)₂NHCORₐ;
Rₐ is a substituted 5-6 membered lactam ring, wherein the substituent for the “substituted” is one or more selected from C1-C6 alkyl;
R₃ is a substituted C3-C7 cycloalkyl, wherein the substituent for the “substituted” is selected from —C(═O)XR_{b} and —(CH₂)ₘOR_{c},
X is —NH— or —O—;
R_{b} is a C1-C6 alkyl, a C1-C6 acyl, or 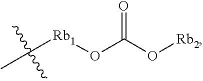
wherein R_{b1} is a C1-C6 alkylene, R_{b2} is a C3-C7 cycloalkyl;
R_{c} is a C1-C6 alkyl, a halogenated C1-C6 alkyl, a C3-C7 cycloalkyl, a C3-C6 alkenyl, a C1-C6 acyl, —CO(CH₂)ₙNR_{d}Rₑ, 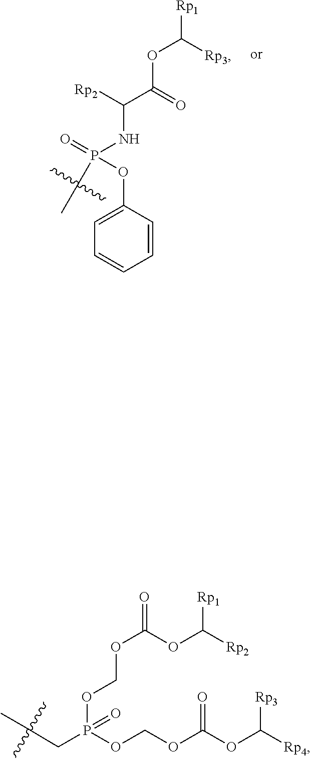
wherein R_{d} and Rₑ are each independently selected from H, C1-C6 alkyl, C3-C7 cycloalkyl, and substituted or unsubstituted C6-C14 aryl; or R_{d} and Rₑ, together with the N atom connected thereto, form a substituted or unsubstituted pyrrolidinyl, piperidyl or piperazinyl, wherein the substituent for the “substituted” is selected from C1-C6 alkyl, halogenated C1-C6 alkyl, —OH, halogen, C1-C6 alkoxy, halogenated C1-C6 alkoxy and —CN;
Rₚ₁, Rₚ₂, Rₚ₃ and Rₚ₄ are each independently a C1-C6 alkyl;
m and n are each independently an integer of 0 to 5;
or
R₁ is a C1-C6 alkyl, a C3-C6 cycloalkyl, or a C1-C2 alkyl substituted by C2-C6 alkenyl,
R₂ is a substituted C6-C14 aryl or a substituted 5-14 membered heteroaryl; the substituent for the “substituted” is selected from C1-C6 alkyl, C1-C6 acyl, —(CH₂)_{q}R_{f}, —(CH₂)_{q}NHCOR_{g}, wherein q is an integer of 2 to 4;
R_{f} is a substituted isoquinolinedione group; R_{g} is a substituted C6-C14 aryl, a substituted 5-14 membered heteroaryl, or a substituted 5-6 membered lactam ring, wherein the substituent for the “substituted” is one or more selected from C1-C6 alkyl, halogenated C1-C6 alkyl, C1-C6 alkoxy, halogenated C1-C6 alkoxy, —OH, halogen, and —CN;
R₃ is a substituted or unsubstituted C1-C6 alkyl, a substituted or unsubstituted C3-C7 cycloalkyl, a substituted or unsubstituted 4-8 membered N-heterocyclyl, a substituted or unsubstituted C3-C6 cycloalkyl-fused pyrrolidinyl, or 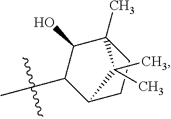
wherein the substituent for the “substituted” is selected from C1-C4 alkyl, —C(═O)XRₕ, and —(CH₂)ₘORᵢ; wherein
X is NH or O;
Rₕ is H, a C1-C6 alkyl, a C1-C6 acyl or 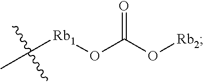
wherein R_{b1} is a C1-C6 alkylene; R_{b2} is a C3-C7 cycloalkyl;
Rᵢ is H, a C1-C6 alkyl, a halogenated C1-C6 alkyl, a C3-C7 cycloalkyl, a C3-C6 alkenyl, a C1-C6 acyl, —CO(CH₂)ₙNR_{d}Rₑ, 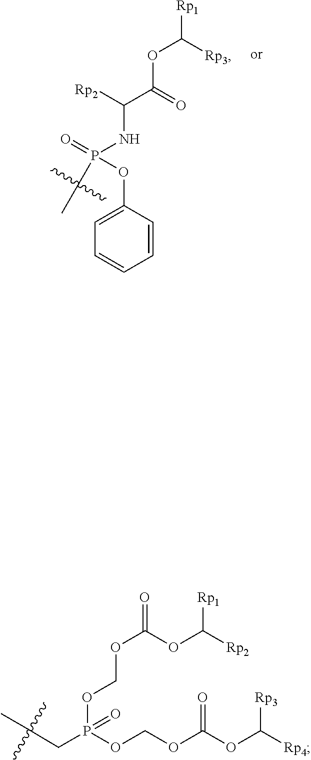
wherein Rₚ₁, Rₚ₂, Rₚ₃ and Rₚ₄ are each independently a C1-C6 alkyl;
R_{d} and Rₑ are each independently selected from H, C1-C6 alkyl, C3-C7 cycloalkyl, and substituted or unsubstituted C6-C14 aryl; or R_{d} and Rₑ, together with the N atom connected thereto, form a substituted or unsubstituted pyrrolidinyl, piperidyl or piperazinyl; wherein the substituent for the “substituted” is selected from C1-C6 alkyl, halogenated C1-C6 alkyl, —OH, halogen, C1-C6 alkoxy, halogenated C1-C6 alkoxy and —CN;
Rₚ₁, Rₚ₂, Rₚ₃, and Rₚ₄ are each independently a C1-C6 alkyl;
m and n are each independently an integer of 0 to 5.

2. The sulfonylurea compound of formula I, or a stereoisomer or pharmaceutically acceptable salt thereof according to claim 1, wherein, the sulfonylurea compound of formula I is selected from the compounds of formulas IA and IB below: 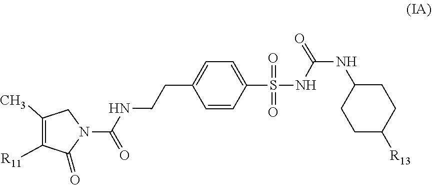
wherein,
R₁₁ is a C1-C6 alkyl; R₁₃ is selected from —C(═O)XR_{b}, and —(CH₂)ₘOR_{c};
wherein X, R_{b}, R_{c} and m are defined as those in claim 1; 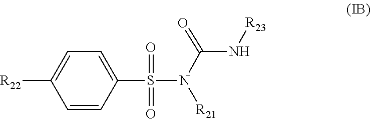
wherein,
R₂₁ is a C1-C6 alkyl, a C3-C6 cycloalkyl, or a C1-C2 alkyl substituted by C2-C6 alkenyl;
R₂₂ is a C1-C6 alkyl, a C1-C6 acyl, —(CH₂)_{q}R_{f} or —(CH₂)_{q}NHCOR_{g}, wherein, q is an integer of 2 to 4;
R₂₃ is a C1-C6 alkyl, a C3-C6 cycloalkyl, a 4-8 membered N-heterocyclyl, a cyclopentyl-fused pyrrolidinyl, 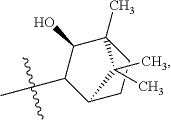
or a substituted cyclohexyl; wherein the substituent for the “substituted” is selected from C1-C4 alkyl, —C(═O)XRₕ, and —(CH₂)ₘORᵢ;
wherein, X, R_{f}, R_{g}, Rₕ and Rᵢ are defined as those in claim 1.

3. The sulfonylurea compound of formula I, or a stereoisomer or pharmaceutically acceptable salt thereof according to claim 2, wherein,
in formula IA,
R_{b} is a C1-C3 alkyl, a C1-C3 acyl, or 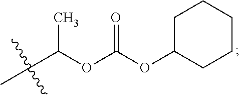 [SMILES: *C(C)OC(=O)OC1CCCCC1]
and/or
R_{c} is a C1-C3 alkyl, a C3-C6 cycloalkyl, a C1-C3 acyl, —CO(CH₂)ₙNR_{d}Rₑ, 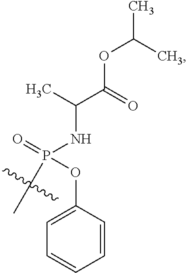 [SMILES: *P(=O)(NC(C)C(=O)OC(C)C)Oc1ccccc1]
or 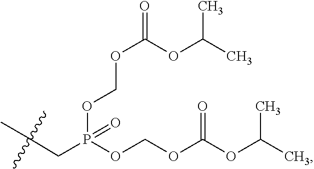
wherein R_{d}, Rₑ and n are defined as those in claim 1; preferably, R_{d} and Rₑ are each independently selected from H, C1-C3 alkyl, C3-C6 cycloalkyl, and substituted or unsubstituted phenyl, or R_{d} and Rₑ, together with the N atom connected thereto, form a substituted or unsubstituted pyrrolidinyl, piperidyl or piperazinyl; the substituent for the “substituted” is selected from C1-C3 alkyl, halogenated C1-C3 alkyl, —OH, halogen, C1-C3 alkoxy, halogenated C1-C3 alkoxy and —CN;
in formula IB,
Rₕ is a C1-C3 alkyl, a C1-C3 acyl, or 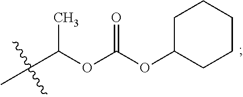 [SMILES: *C(C)OC(=O)OC1CCCCC1]
and/or
Rᵢ is a C1-C3 alkyl, a C3-C6 cycloalkyl, a C1-C3 acyl, —CO(CH₂)ₙNR_{d}Rₑ, 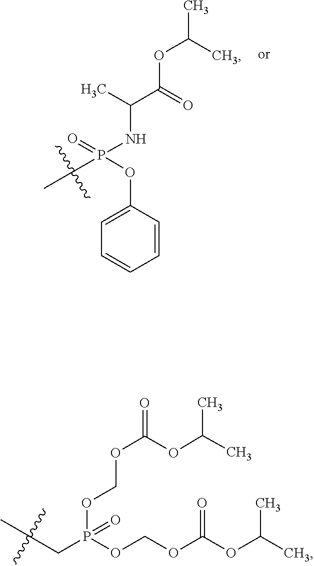
wherein R_{d}, Rₑ and n are defined as those in claim 1; preferably, R_{d} and Rₑare each independently selected from H, C1-C3 alkyl, C3-C6 cycloalkyl, and substituted or unsubstituted phenyl, or R_{d} and Rₑ, together with the N atom connected thereto, form a substituted or unsubstituted pyrrolidinyl, piperidyl or piperazinyl; the substituent for the “substituted” is selected from C1-C3 alkyl, halogenated C1-C3 alkyl, —OH, halogen, C1-C3 alkoxy, halogenated C1-C3 alkoxy and —CN.

4. The sulfonylurea compound of formula I, or a stereoisomer or pharmaceutically acceptable salt thereof according to claim 2, wherein, the compound of formula IB is selected from the compounds of formulas IB1-IB7 below: 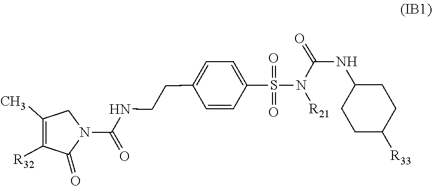
wherein,
R₂₁ is a C1-C6 alkyl, a C3-C6 cycloalkyl, or a C1-C2 alkyl substituted by C2-C6 alkenyl;
R₃₂ is a C1-C6 alkyl;
R₃₃ is a C1-C4 alkyl, —C(═O)XRₕ, or —(CH₂)ₘORᵢ;
X is NH or O;
Rₕ is H, a C1-C3 alkyl or 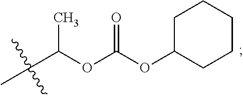 [SMILES: *C(C)OC(=O)OC1CCCCC1]
Rᵢ is H, a C1-C6 alkyl, a halogenated C1-C6 alkyl, a C3-C6 cycloalkyl, a C3-C6 alkenyl, a C1-C6 acyl, —CO(CH₂)ₙNR_{d}Rₑ, 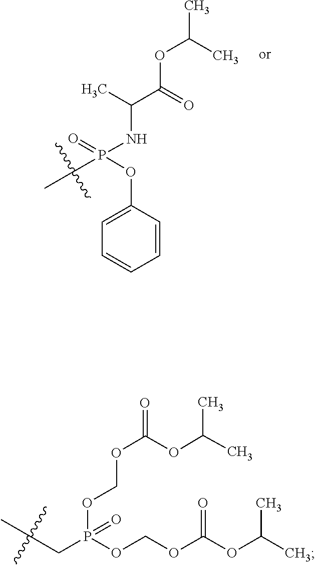
R_{d} and Rₑ are each independently selected from H, C1-C3 alkyl, C3-C6 cycloalkyl, or substituted or unsubstituted C6-C14 aryl; or R_{d} and Rₑ, together with the N atom connected thereto, form a substituted or unsubstituted pyrrolidinyl, piperidyl or piperazinyl; wherein the substituent for the “substituted” is selected from C1-C3 alkyl, halogenated C1-C3 alkyl, —OH, halogen, C1-C3 alkoxy, halogenated C1-C3 alkoxy and —CN;
m and n are each independently an integer of 0 to 5; 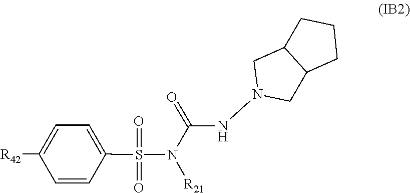
wherein,
R₂₁ is a C1-C6 alkyl, a C3-C6 cycloalkyl, or a C1-C2 alkyl substituted by C2-C6 alkenyl;
R₄₂ is a C1-C6 alkyl; 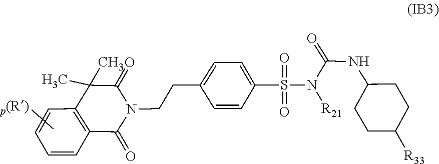
wherein,
R′ is each independently a C1-C6 alkyl or a C1-C6 alkoxy; p is an integer of 1 to 4;
R₂₁ is a C1-C6 alkyl, a C3-C6 cycloalkyl, or a C1-C2 alkyl substituted by C2-C6 alkenyl;
R₃₃ is H, a C1-C4 alkyl, —C(═O)XRₕ, or —(CH₂)ₘORᵢ;
X is NH or O;
Rₕ is H, a C1-C6 alkyl, or 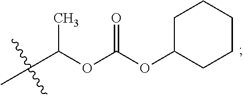 [SMILES: *C(C)OC(=O)OC1CCCCC1]
Rᵢ is H, a C1-C6 alkyl, a halogenated C1-C6 alkyl, a C3-C6 cycloalkyl, a C3-C6 alkenyl, a C1-C6 acyl, —CO(CH₂)ₙNR_{d}Rₑ, 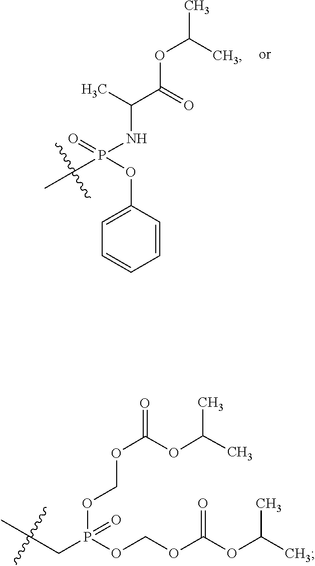
R_{d} and Rₑ are each independently selected from H, C1-C3 alkyl, C3-C6 cycloalkyl, and substituted or unsubstituted C6-C14 aryl, or R_{d} and Rₑ, together with the N atom connected thereto, form a substituted or unsubstituted pyrrolidinyl, piperidyl or piperazinyl; wherein the substituent for the “substituted” is selected from C1-C3 alkyl, halogenated C1-C3 alkyl, —OH, halogen, C1-C3 alkoxy, halogenated C1-C3 alkoxy and —CN;
m and n are independent integers of 0 to 5; 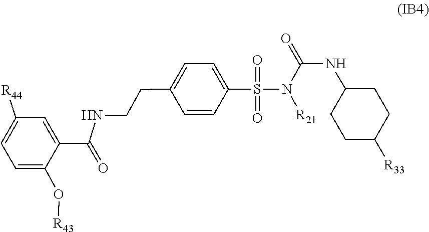
wherein,
R₂₁ is a C1-C6 alkyl, a C3-C6 cycloalkyl, or a C1-C2 alkyl substituted by C2-C6 alkenyl;
R₃₃ is selected from H, a C1-C4 alkyl, —C(═O)XRₕ, and —(CH₂)ₘORᵢ;
X is NH or O;
Rₕ is H, C1-C3 alkyl, or 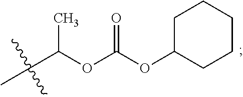 [SMILES: *C(C)OC(=O)OC1CCCCC1]
Rᵢ is H, a C1-C6 alkyl, a halogenated C1-C6 alkyl, a C3-C6 cycloalkyl, a C3-C6 alkenyl, a C1-C6 acyl, —CO(CH₂)ₙNR_{d}Rₑ, 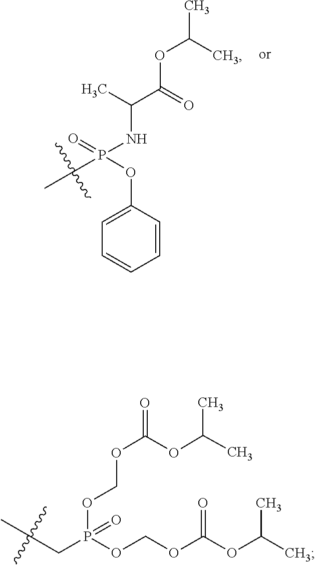
R_{d} and Rₑ are each independently selected from H, C1-C3 alkyl, C3-C6 cycloalkyl, substituted or unsubstituted C6-C14 aryl; or R_{d} and Rₑ, together with the N atom connected thereto, form substituted or unsubstituted pyrrolidinyl, piperidyl or piperazinyl; wherein the substituent for the “substituted” is selected from C1-C3 alkyl, halogenated C1-C3 alkyl, —OH, halogen, C1-C3 alkoxy, halogenated C1-C3 alkoxy and —CN;
m and n are each independently an integer of 0 to 5;
R₄₃ is a C1-C6 alkyl;
R₄₄ is a halogen; 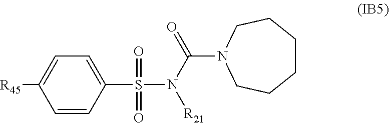
wherein,
R₂₁ is a C1-C6 alkyl, a C3-C6 cycloalkyl, or a C1-C2 alkyl substituted by C2-C6 alkenyl;
R₄₅ is a C1-C6 alkyl or —(CH₂)_{q}NHCOR_{g}; R_{g} is a substituted 5-14 membered heteroaryl; q is an integer of 2-4; the preferred heteroaryl is isoxazolyl; 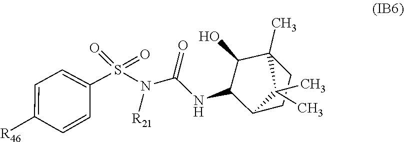
wherein,
R₂₁ is a C1-C6 alkyl, a C3-C6 cycloalkyl, or a C1-C2 alkyl substituted by C2-C6 alkenyl;
R₄₆ is a C1-C6 alkyl; 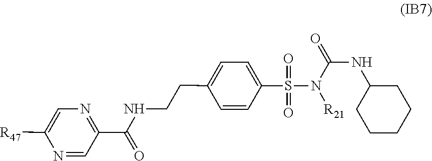
wherein,
R₂₁ is a C1-C6 alkyl, a C3-C6 cycloalkyl, or a C1-C2 alkyl substituted by C2-C6 alkenyl;
R₄₇ is a C1-C6 alkyl.

5. The sulfonylurea compound of formula I, or a stereoisomer or pharmaceutically acceptable salt thereof according to claim 1, wherein, the sulfonylurea compound is selected from the following compounds:

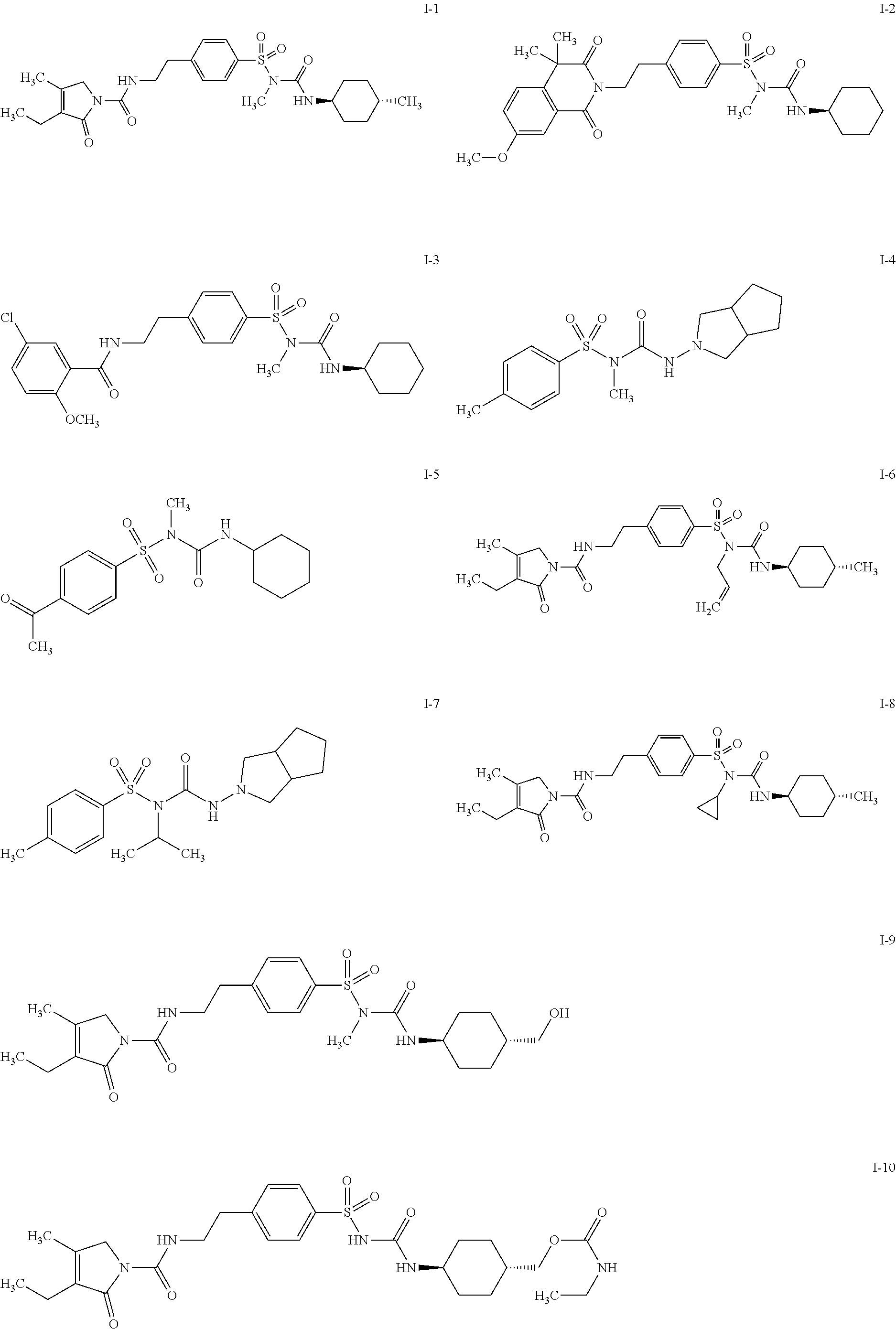

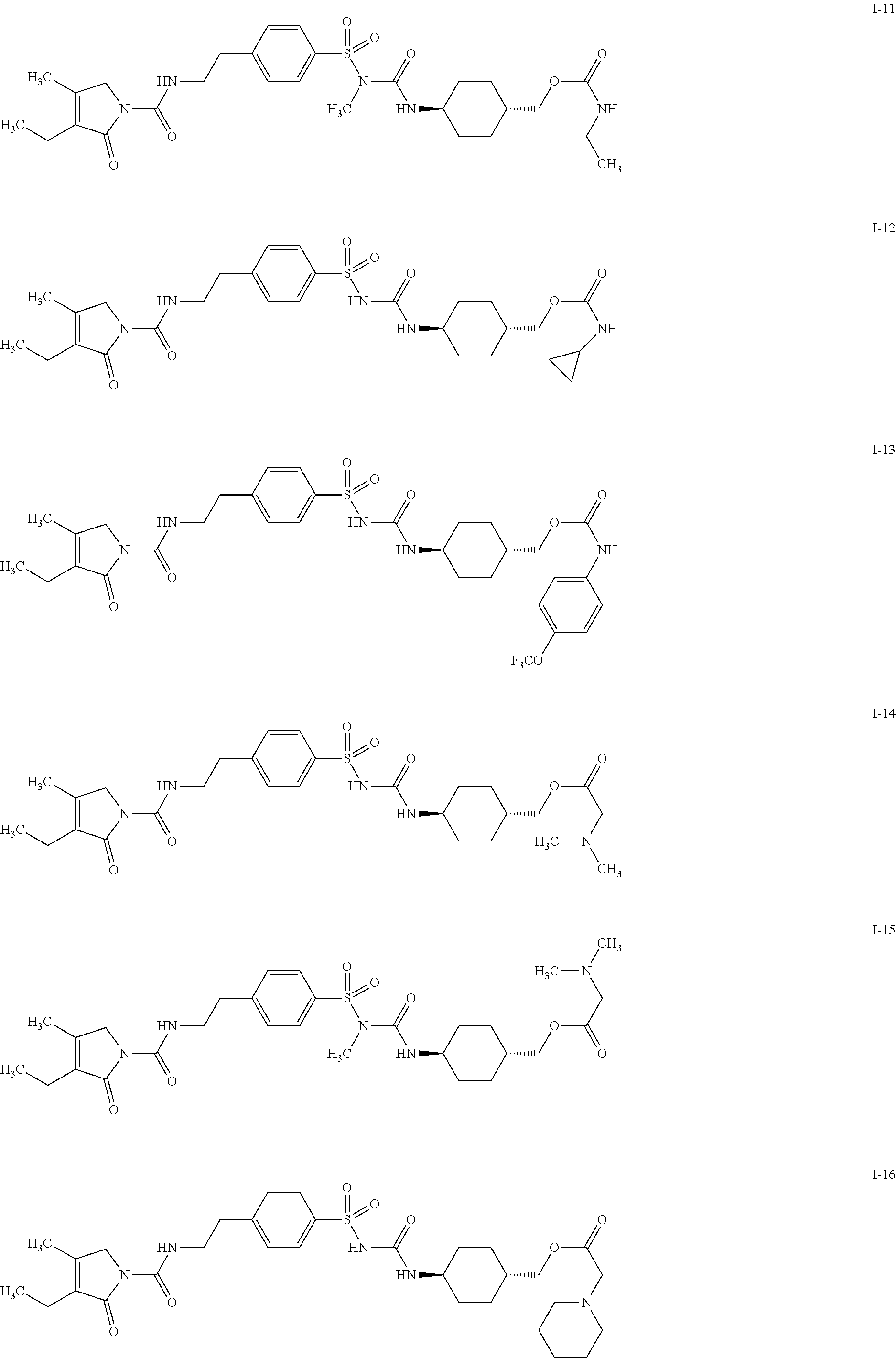

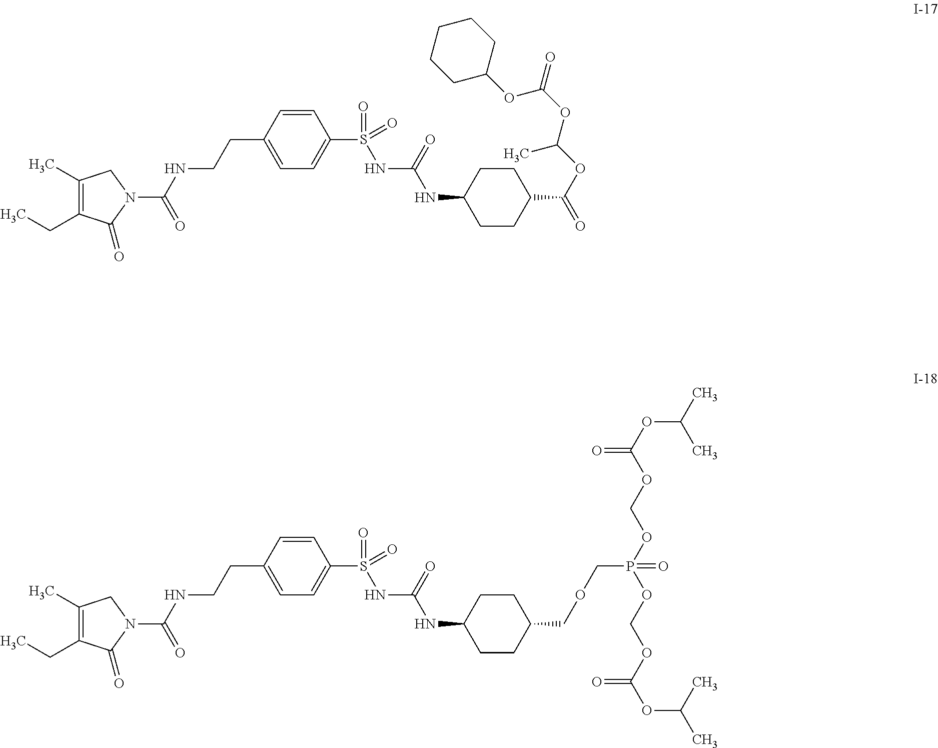

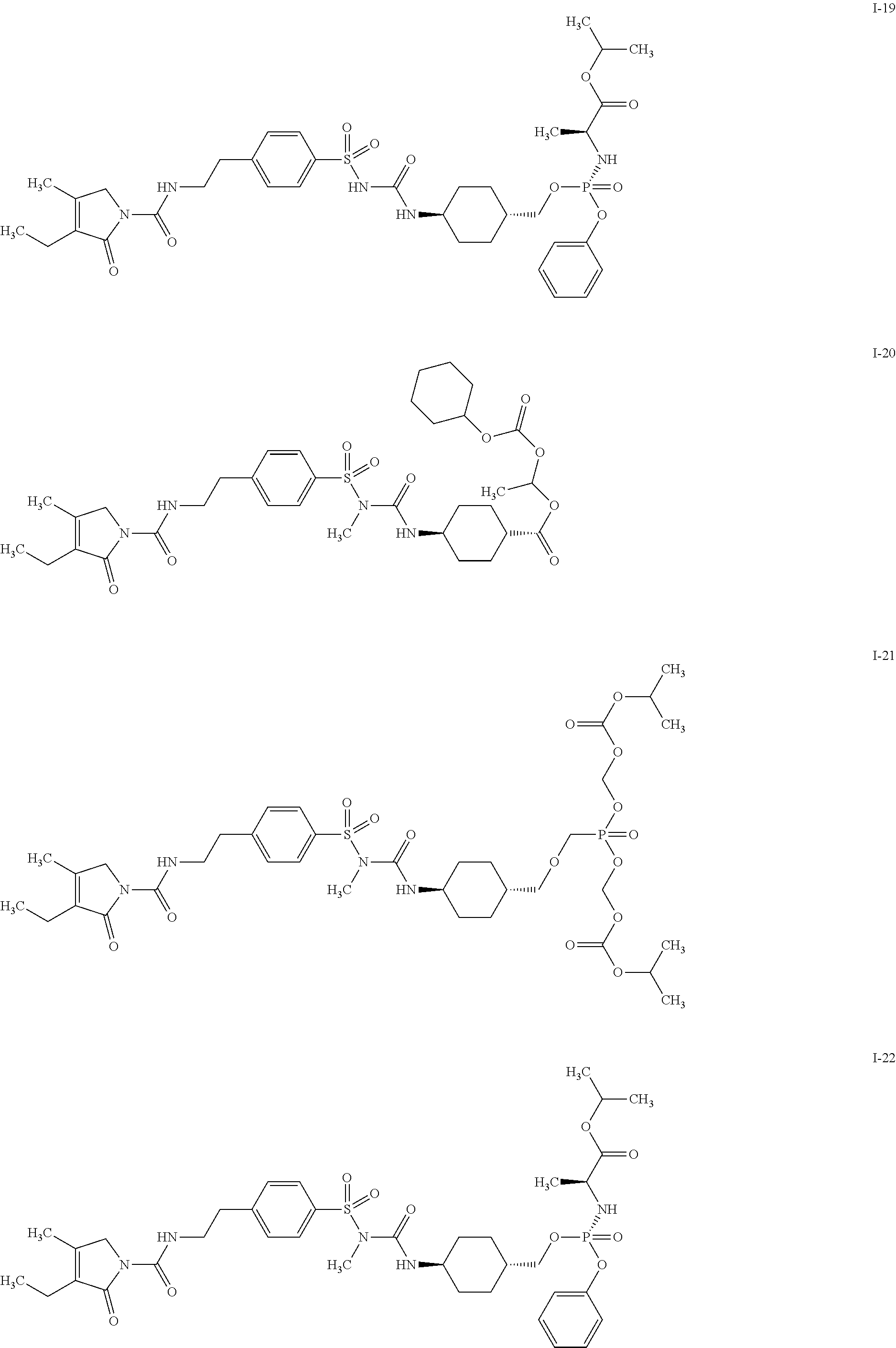

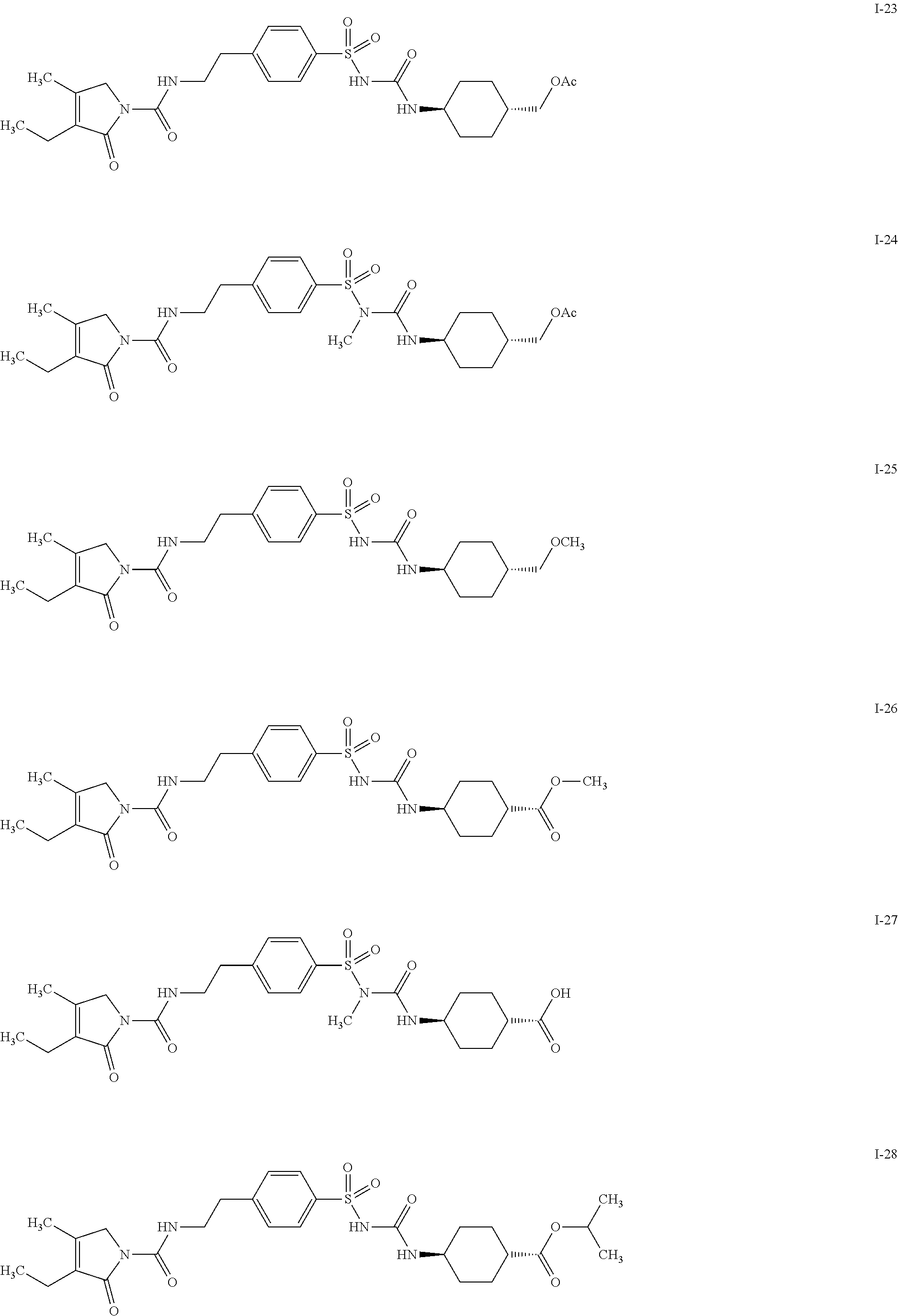

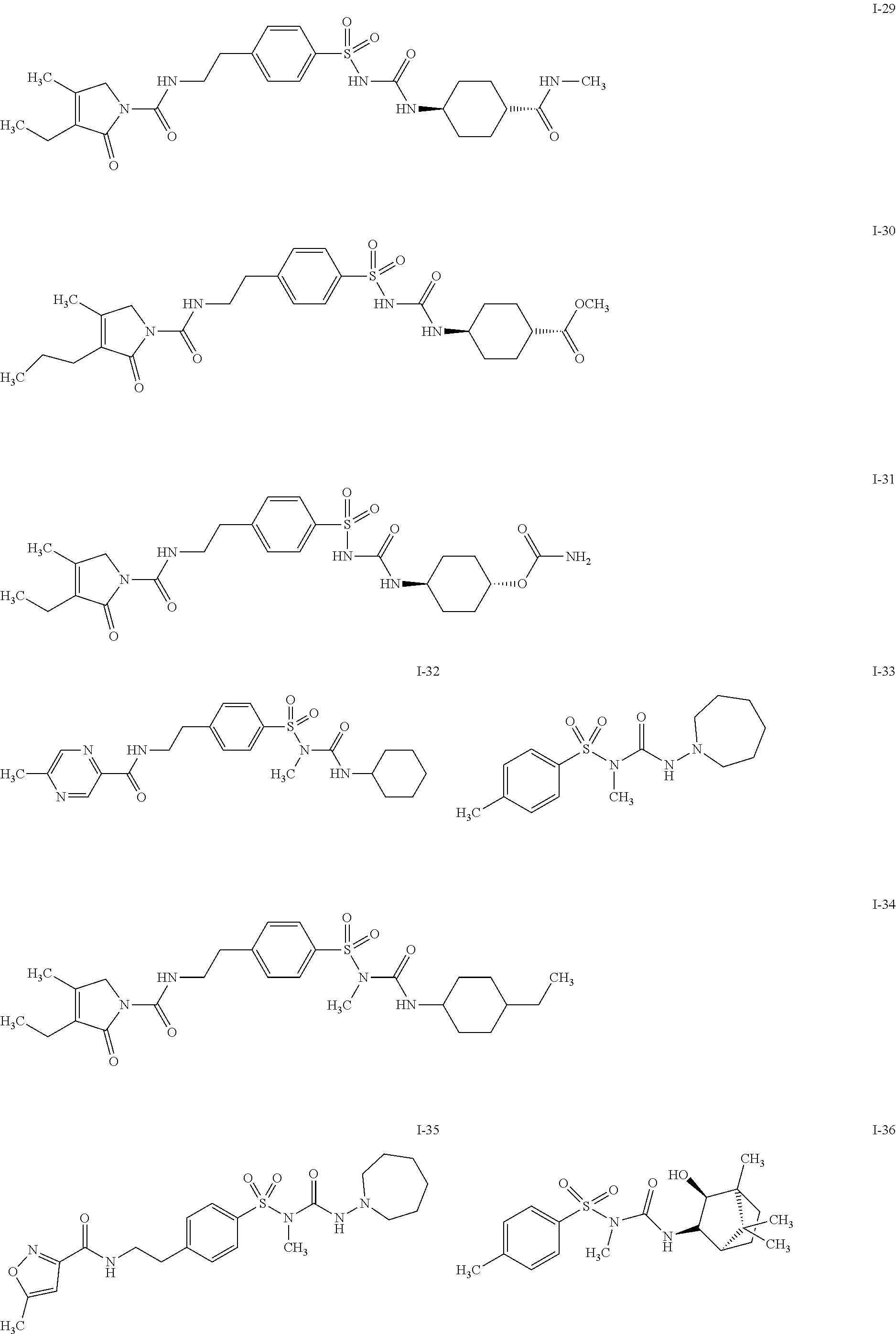

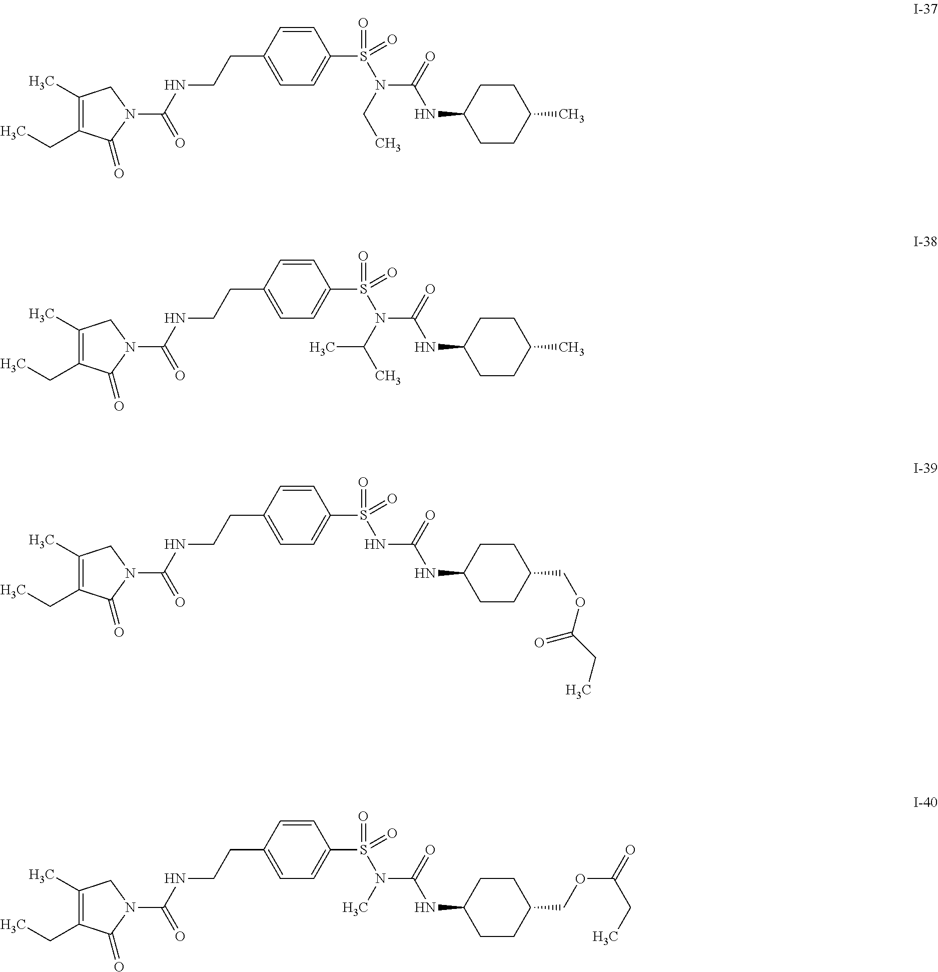

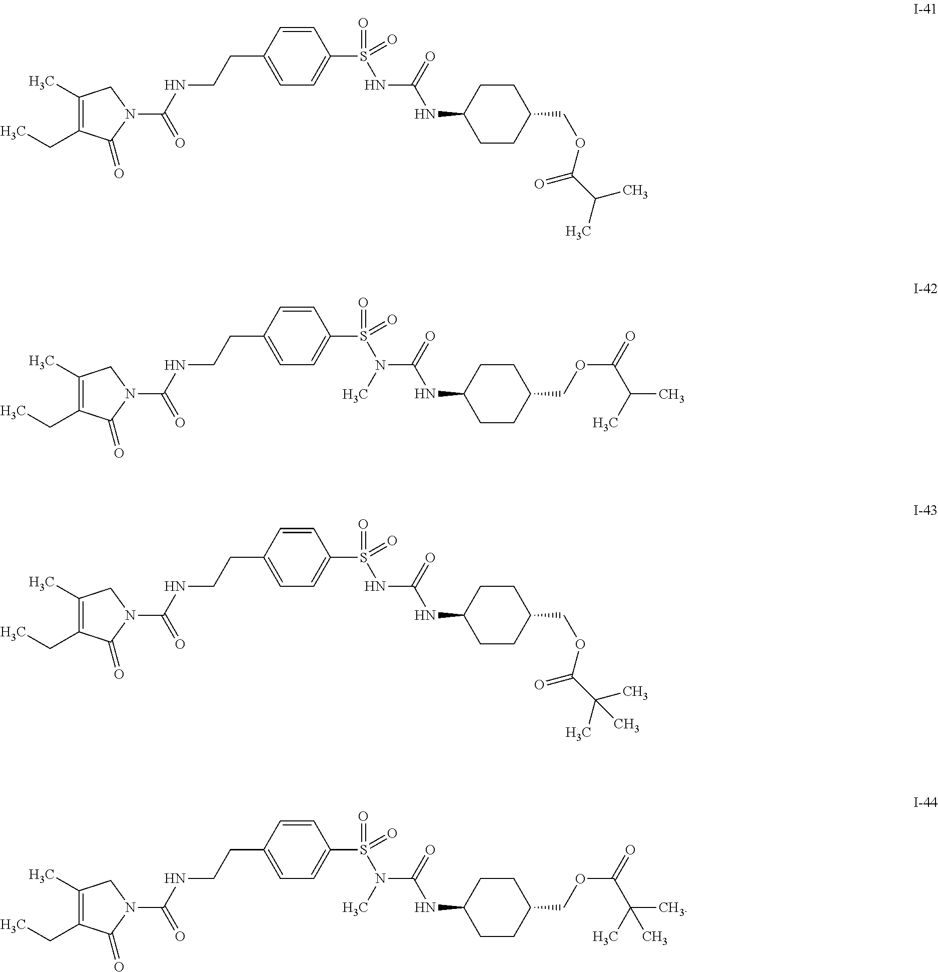

6. A method for preparing the sulfonylurea compound according to claim 2, which is one of the following methods:
Method 1: 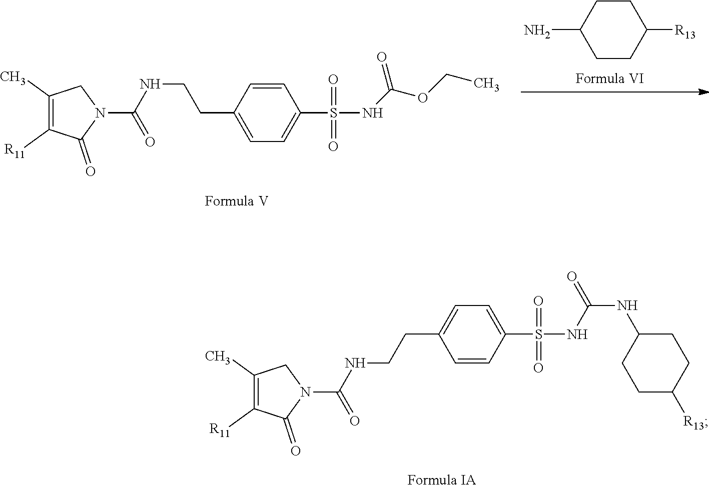
the compound of formula V reacts with the compound of formula VI through nucleophilic substitution to obtain the compound of formula IA;
Method 2: 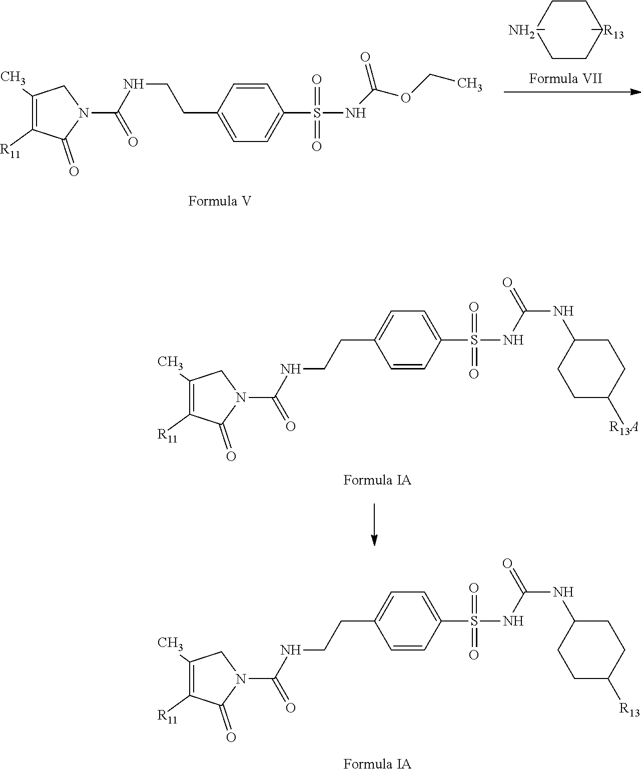
the compound of formula VII reacts with the compound of formula V through nucleophilic substitution to obtain the compound of formula VIII, which is then subjected to esterification, acylation, etherification, or phosphorylation to obtain the compound of Formula IA;
wherein, R_{13A} is —COOH or —(CH₂)ₘOH; and R₁₁, R₁₃ and m are defined as those in claim 2;
Method 3: 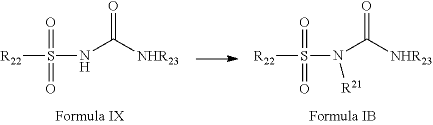
the compound of formula IX reacts with R₂₁—I through alkylation to obtain the compound of formula IB,
wherein, R₂₁, R₂₂ and R₂₃ are defined as those in claim 2.

7. A pharmaceutical composition comprising one or more selected from the group consisting of the sulfonylurea compound and the stereoisomer and pharmaceutically acceptable salt thereof according to claim 1; and optionally, one or more pharmaceutically acceptable excipients, diluents, carriers, excipients, or adjuvants.

8. A soluble epoxide hydrolase inhibitor comprising one or more selected from the group consisting of the sulfonylurea compound and the stereoisomer and pharmaceutically acceptable salt thereof according to claim 1.

9. A method comprising administering to a subject in need thereof an effective amount of the sulfonylurea compound or the stereoisomer or pharmaceutically acceptable salt thereof according to claim 1, wherein the method is selected from the group consisting of: inhibiting the activity of a soluble epoxide hydrolase; increasing the level of epoxyeicosatrienoic acids; reducing inflammatory response; and preventing and/or treating heart failure.

10. A sulfonylurea drug for prevention and/or treatment of heart failure, wherein the sulfonylurea drug is selected from the group consisting of glimepiride, gliclazide, gliquidone, glibenclamide, glipizide, tolazamide, torasemide and acetohexamide.
